SEQUENCE LISTING (17 sequences)

SEQ ID NO: 1 - protein, 5 aa, synthetic construct
DYGMV

SEQ ID NO: 2 - protein, 17 aa, synthetic construct
FISSGSSTVYYADTMKG

SEQ ID NO: 3 - protein, 10 aa, synthetic construct
RNYDDWYFDV

SEQ ID NO: 4 - protein, 10 aa, synthetic construct
SASSSVTYMY

SEQ ID NO: 5 - protein, 7 aa, synthetic construct
RTSDLAS

SEQ ID NO: 6 - protein, 9 aa, synthetic construct
QHYHSYPLT

SEQ ID NO: 7 - protein, 119 aa, synthetic construct
EVQLVESGGGLVQPGGSLRLSCAASGFTFSDYGMVWVRQAPGKGLEWVAFISSGSSTVYYADTMKGRFTISRDNSKNTLYLQMNSLRAEDTAVYYCTRRNYDDWYFDVWGQGTLVTVSS

SEQ ID NO: 8 - protein, 106 aa, synthetic construct
DIQMTQSPSSLSASVGDRVTITCSASSSVTYMYWYQQKPGKSPKPWIYRTSDLASGVPSRFSGSGSGTDFTLTISSLQPEDFATYYCQHYHSYPLTFGQGTKVEIK

SEQ ID NO: 9 - protein, 448 aa, synthetic construct
EVQLVESGGGLVQPGGSLRLSCAASGFTFSDYGMVWVRQAPGKGLEWVAFISSGSSTVYYADTMKGRFTISRDNSKNTLYLQMNSLRAEDTAVYYCTRRNYDDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 213 aa, synthetic construct
DIQMTQSPSSLSASVGDRVTITCSASSSVTYMYWYQQKPGKSPKPWIYRTSDLASGVPSRFSGSGSGTDFTLTISSLQPEDFATYYCQHYHSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 445 aa, synthetic construct
EVQLVESGGGLVQPGGSLRLSCAASGFTFSDYGMVWVRQAPGKGLEWVAFISSGSSTVYYADTMKGRFTISRDNSKNTLYLQMNSLRAEDTAVYYCTRRNYDDWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 275 aa, Homo sapiens
MLNLLLLALPVLASRAYAAPAPGQALQRVGIVGGQEAPRSKWPWQVSLRVHGPYWMHFCGGSLIHPQWVLTAAHCVGPDVKDLAALRVQLREQHLYYQDQLLPVSRIIVHPQFYTAQIGADIALLELEEPVNVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKP

SEQ ID NO: 13 - protein, 275 aa, Homo sapiens
MLNLLLLALPVLASRAYAAPAPGQALQRVGIVGGQEAPRSKWPWQVSLRVHGPYWMHFCGGSLIHPQWVLTAAHCVGPDVKDLAALRVQLREQHLYYQDQLLPVSRIIVHPQFYTAQIGADIALLELEEPVKVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKP

SEQ ID NO: 14 - protein, 233 aa, Homo sapiens
MLNLLLLALPVLASRAYAAPAPGQALQRVGIVGGQEAPRSKWPWQVSLRVRDRYWMHFCGGSLIHPQWVLTAAHCVGPDVKDLAALRVQLREQHLYYQDQLLPVSRIIVHPQFYTAQIGADIALLELEEPVNVSSHVHTVTLPPASETFPPGMPCWVTGWGDVDNDERLPPPFPLKQVKVPIMENHICDAKYHLGAYTGDDVRIVRDDMLCAGNTRRDSCQVATAPHTFPAPS

SEQ ID NO: 15 - protein, 245 aa, Homo sapiens
IVGGQEAPRSKWPWQVSLRVHGPYWMHFCGGSLIHPQWVLTAAHCVGPDVKDLAALRVQLREQHLYYQDQLLPVSRIIVHPQFYTAQIGADIALLELEEPVNVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKP

SEQ ID NO: 16 - protein, 256 aa, Homo sapiens
IVGGQEAPRSKWPWQVSLRVHGPYWMHFCGGSLIHPQWVLTAAHCVGPDVKDLAALRVQLREQHLYYQDQLLPVSRIIVHPQFYTAQIGADIALLELEEPVKVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKPGNSDYKDDDDK

SEQ ID NO: 17 - protein, 256 aa, Homo sapiens
IVGGQEAPRSKWPWQVSLRVRDRYWMHFCGGSLIHPQWVLTAAHCVGPDVKDLAALRVQLREQHLYYQDQLLPVSRIIVHPQFYTAQIGADIALLELEEPVNVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKPGNSDYKDDDDK

(19) United States
(12) Patent Application Publication
(10) Pub. No.: US 2024/0270873 A1
(43) Pub. Date: Aug. 15, 2024

(54) DOSING FOR ANTI-TRYPTASE ANTIBODIES

(71) Applicant: Genentech, Inc., South San Francisco, CA (US)

(72) Inventors: Sara Beth GLICKSTEIN BAR-ZEEV, Oakland, CA (US); Horace H. RHEE, San Mateo, CA (US); Sharon Marie RYMUT, San Francisco, CA (US); Tracy Lyn STATON, Stanford, CA (US); Kenta YOSHIDA, Foster City, CA (US); David Fong CHOY, Fairfield, CA (US)

(21) Appl. No.: 18/438,661

(22) Filed: Feb. 12, 2024

Related U.S. Application Data
(63) Continuation of application No. PCT/US22/74900, filed on Aug. 12, 2022.
(60) Provisional application No. 63/233,094, filed on Aug. 13, 2021.

(51) Int. Cl. C07K 16/40; A61K 9/00; A61K 39/00; A61K 45/06; A61P 17/04

(52) U.S. Cl. CPC C07K 16/40; A61K 9/0019; A61K 45/06; A61P 17/04; A61K 2039/505; A61K 2039/545; C07K 2317/565

(57) ABSTRACT

The present invention features, inter alia, methods of treating a patient having chronic spontaneous urticaria (CSU) by administering an anti-tryptase antibody (e.g., anti-tryptase beta antibody) to the patient, anti-tryptase antibodies (e.g., anti-tryptase beta antibodies) for use in treating CSU, and uses of anti-tryptase antibodies (e.g., anti-tryptase beta antibodies), e.g., in the manufacture of medicaments for treating CSU.

### SEQUENCE LISTING

The instant application contains a Sequence Listing which has been submitted electronically in XML format and is hereby incorporated by reference in its entirety. Said XML copy, created on Feb. 8, 2024, is named 50474-263003_Sequence_Listing_2_8_24 and is 18,496 bytes in size.

### FIELD OF THE INVENTION

The present disclosure relates to methods of treating chronic spontaneous urticaria (CSU) and related compositions and uses.

### BACKGROUND

Chronic spontaneous urticaria (CSU), also referred to as chronic idiopathic urticaria (CIU), is typically defined by the presence of wheals (hives), angioedema, or both for at least six weeks without an obvious cause. Previous estimates of the prevalence of CSU were approximately 0.1%, persisting in 20% of CSU patients two decades after diagnosis. More recent evidence indicates that the point prevalence of the disease is approximately 1%. Affected patients experience frequent pruritic hives with associated erythema and/or episodes of angioedema; CSU is reported to be associated with angioedema in approximately 50% of cases. The classic urticaria description is a wheal and flare with a pale, elevated lesion and surrounding erythema. Urticaria range in size from a few millimeters to a few centimeters across and usually occur in groups that often coalesce into large confluent lesions.

The etiology of CSU is not clear. Among several theories, both infectious origins and autoimmune origins have been considered. Some studies have found that 30%-60% of patients with CSU have a positive autologous serum skin test, suggesting an autoimmune etiology. In addition, CSU has been theorized to result from the activation of skin mast cells after high-affinity IgE receptor (FcεRI) binding of IgE antibody targeted against an endogenous antigen. Activated skin mast cells release chemical mediators, such as histamine and tryptase, which lead to the wheal and flare formation of a hive and pruritus. In fact, one study demonstrated that approximately 50% of patients with CSU had IgE antibodies directed against thyroperoxidase. Nonetheless, in many patients, this autoimmune link cannot be drawn despite similar disease presentation.

Improved therapies for CSU are still being sought.

### SUMMARY OF THE INVENTION

The present invention features, inter alia, methods of treating a patient having CSU (e.g., CSU refractory to antihistamines (e.g., second-generation H1 antihistamines (sgH1-AHs))), anti-tryptase antibodies (e.g., anti-tryptase beta antibodies) for use in treating CSU, uses of anti-tryptase antibodies (e.g., anti-tryptase beta antibodies), e.g., in the manufacture of medicaments for treating CSU, as well as related kits and articles of manufacture.

In one aspect, the invention features a method of treating a patient having CSU, the method comprising administering to a patient having CSU an anti-tryptase beta antibody in a dosing regimen comprising a dosing cycle, wherein the dosing cycle comprises a first dose (C1D1) of the anti-tryptase beta antibody selected from 300 mg subcutaneously (SC), 600 mg SC, 900 mg intravenously (IV), or 1800 mg IV, wherein the anti-tryptase beta antibody comprises the following six complementarity determining regions (CDRs): (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, the invention features a method of treating a patient having CSU, the method comprising administering to a patient having CSU an anti-tryptase beta antibody in a dosing regimen comprising a dosing cycle, wherein the dosing cycle comprises between two and six doses, wherein a total of about 300 mg to about 21,600 mg of the anti-tryptase antibody is administered SC or IV to the patient in the dosing cycle, and wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, the invention features a method of treating a patient having CSU, the method comprising administering to a patient having CSU an anti-tryptase beta antibody in a dosing regimen comprising a dosing cycle, wherein the dosing cycle comprises administering the anti-tryptase beta antibody at a dose selected from 300 mg SC, 600 mg SC, 900 mg IV, or 1800 mg IV every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, the invention features a kit comprising an anti-tryptase beta antibody and instructions to administer the anti-tryptase beta antibody to a patient having CSU in accordance with any one of the methods disclosed herein.

In another aspect, the invention features an anti-tryptase beta antibody for use in treating a patient having CSU, wherein the anti-tryptase beta antibody is for administration to a patient having CSU in a dosing regimen comprising a dosing cycle, wherein the dosing cycle comprises a first dose (C1D1) of the anti-tryptase beta antibody selected from 300 mg SC, 600 mg SC, 900 mg IV, or 1800 mg IV, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, the invention features an anti-tryptase beta antibody for use in treating a patient having CSU, wherein the anti-tryptase beta antibody is for administration to a patient having CSU in a dosing regimen comprising a dosing cycle, wherein the dosing cycle comprises between two and six doses, wherein a total of about 300 mg to about 21,600 mg of the anti-tryptase antibody is administered SC or IV to the patient in the dosing cycle, and wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, the invention features an anti-tryptase beta antibody for use in treating a patient having CSU, wherein the anti-tryptase beta antibody is for administration to a patient having CSU in a dosing regimen comprising a dosing cycle, wherein the dosing cycle comprises administering the anti-tryptase beta antibody at a dose selected from 300 mg SC, 600 mg SC, 900 mg IV, or 1800 mg IV every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, the invention features use of an anti-tryptase beta antibody in the manufacture of a medicament for treating a patient having CSU, wherein the medicament is for administration to a patient having CSU in a dosing regimen comprising a dosing cycle, wherein the dosing cycle comprises a first dose (C1D1) of the anti-tryptase beta antibody selected from 300 mg SC, 600 mg SC, 900 mg IV, or 1800 mg IV, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, the invention features use of an anti-tryptase beta antibody in the manufacture of a medicament for treating a patient having CSU, wherein the medicament is for administration to a patient having CSU in a dosing regimen comprising a dosing cycle, wherein the dosing cycle comprises between two and six doses, wherein a total of about 300 mg to about 21,600 mg of the anti-tryptase antibody is administered SC or IV to the patient in the dosing cycle, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, the invention features use of an anti-tryptase beta antibody in the manufacture of a medicament for treating a patient having CSU, wherein the medicament is for administration to a patient having CSU in a dosing regimen comprising a dosing cycle, wherein the dosing cycle comprises administering the anti-tryptase beta antibody at a dose selected from 300 mg SC, 600 mg SC, 900 mg IV, or 1800 mg IV every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In some aspects, the antibody comprises (a) a heavy chain variable (VH) domain comprising an amino acid sequence having at least 90%, at least 95%, or at least 99% sequence identity to the amino acid sequence of SEQ ID NO: 7; (b) a light chain variable (VL) domain comprising an amino acid sequence having at least 90%, at least 95%, or at least 99% identity to the amino acid sequence of SEQ ID NO: 8; or (c) a VH domain as in (a) and a VL domain as in (b).

In some aspects, the VH domain comprises the amino acid sequence of SEQ ID NO: 7.

In some aspects, the VL domain comprises the amino acid sequence of SEQ ID NO: 8.

In some aspects, the VH domain comprises the amino acid sequence of SEQ ID NO: 7 and the VL domain comprises the amino acid sequence of SEQ ID NO: 8.

In some aspects, the antibody comprises (a) a heavy chain comprising the amino acid sequence of SEQ ID NO: 9 and (b) a light chain comprising the amino acid sequence of SEQ ID NO: 10.

In some aspects, the antibody comprises (a) a heavy chain comprising the amino acid sequence of SEQ ID NO: 11 and (b) a light chain comprising the amino acid sequence of SEQ ID NO: 10.

In some aspects, the C1D1 is 300 mg SC.

In some aspects, the C1D1 is 600 mg SC.

In some aspects, the C1D1 is 900 mg IV.

In some aspects, the C1D1 is 1800 mg IV.

In some aspects, the dosing cycle further comprises a second dose (C1D2) and a third dose (C1D3) of the anti-tryptase beta antibody, wherein the C1D2 and the C1D3 are each equal to the C1D1.

In some aspects, the doses of the dosing cycle are administered to the subject every four weeks (Q4W).

In some aspects, the dosing cycle has a length of about 57 days.

In some aspects, the C1D1 is administered on Day 1 of the dosing cycle, the C1D2 is administered on Day 29 (±1 day) of the dosing cycle, and the C1D3 is administered on Day 57 (±1 day) of the dosing cycle.

In some aspects, the dosing regimen consists of one dosing cycle.

In some aspects, the CSU is refractory to antihistamines.

In some aspects, the CSU is refractory to sgH1-AHs.

In some aspects, the patient: (i) has had a CSU diagnosis for greater than or equal to (≥) 6 months; (ii) has presence of itch and hives for greater than (>) 6 consecutive weeks at any time prior to treatment despite current use of sgH1-AHs, consistent with standard of care during this time period; (iii) has received stable doses of sgH1-AHs, consistent with standard of care therapy for CSU, starting at least 14 (−4/+2 days) consecutive days prior to treatment; and/or (iv) has a Urticaria Activity Score summed over 7 days (UAS7) symptom score of ≥16 during the 7 days prior to the C1D1.

In some aspects, the patient has a UAS7 symptom score of ≥16.

In some aspects, the patient is Chronic Urticaria Index (CU Index®)-positive.

In some aspects, the patient is receiving background sgH1-AH therapy.

In some aspects, the background sgH1-AH therapy comprises cetirizine 10-40 mg QD, levocetirizine 5-20 mg QD, fexofenadine 180-720 mg QD, loratadine 10-40 mg QD, desloratadine 5-20 mg QD, rupatadine 10-40 mg QD, or bilastine 20-80 mg QD.

In some aspects, the patient receives a single dose of rescue therapy within a 24-hour period if symptoms worsen.

In some aspects, the rescue therapy comprises up to 10 mg loratadine or up to 10 mg cetirizine.

In some aspects, the treating results in an improvement from baseline in the patient's UAS7 at Week 12 compared to placebo.

In some aspects, (i) the treatment results in well-controlled urticaria (UAS7 less than or equal to (≤) 6 at Week 12); or (ii) the treatment results in a complete response (UAS7=0) at Week 12.

### BRIEF DESCRIPTION OF THE DRAWINGS

FIG. 1 is a schematic diagram of the study design of the GA43512 Phase II clinical study. IV, intravenous; Q4W, every four weeks; SC, subcutaneous.

### DETAILED DESCRIPTION OF ASPECTS OF THE INVENTION

### I. Introduction

The present invention provides therapeutic methods and compositions for CSU (e.g., CSU refractory to antihistamines (e.g., sgH1-AHs)). The present invention is based, at least in part, on the development of dosing regimens for anti-tryptase antibodies suitable for treatment of CSU, e.g., as described in Example 1.

### II. Definitions

The term “about,” as used herein, refers to the usual error range for the respective value readily known to the skilled person in this technical field. Reference to “about” a value or parameter herein includes (and describes) aspects that are directed to that value or parameter per se.

As used herein, “tryptase” refers to any native tryptase from any vertebrate source, including mammals such as primates (e.g., humans) and rodents (e.g., mice and rats), unless otherwise indicated. Tryptase is also known in the art as mast cell tryptase, mast cell protease II, skin tryptase, lung tryptase, pituitary tryptase, mast cell neutral proteinase, and mast cell serine proteinase II. The term “tryptase” encompasses tryptase alpha (encoded in humans by TPSAB1), tryptase beta (encoded in humans by TPSAB1 and TPSB2; see below), tryptase delta (encoded in humans by TPSD1), tryptase gamma (encoded in humans by TPSG1), and tryptase epsilon (encoded in humans by PRSS22). Tryptase alpha (a), beta (B), and gamma (γ) proteins are soluble, whereas tryptase epsilon (ε) proteins are membrane anchored. Tryptase beta and gamma are active serine proteases, although they have different specificities. Tryptase alpha and delta (δ) proteins are largely inactive proteases as they have residues in critical position that differ from typical active serine proteases. An exemplary tryptase alpha full-length protein sequence can be found under NCBI GenBank Accession No. ACZ98910.1. Exemplary tryptase gamma full-length protein sequences can be found under Uniprot Accession No. Q9NRR2 or GenBank Accession Nos. Q9NRR2.3, AAF03695.1, NP_036599.3 or AAF76457.1. Exemplary tryptase delta full-length protein sequences can be found under Uniprot Accession No. Q9BZJ3 or GenBank Accession No. NP_036349.1. Several tryptase genes are clustered on human chromosome 16p13.3. The term encompasses “full-length,” unprocessed tryptase as well as any form of tryptase that results from processing in the cell. Tryptase beta is the main tryptase expressed in mast cells, while tryptase alpha is the main tryptase expressed in basophils. Tryptase alpha and tryptase beta typically include a leader sequence of approximately 30 amino acids and a catalytic sequence of approximately 245 amino acids (see, e.g., Schwartz, Immunol. Allergy Clin. N. Am. 26:451-463, 2006).

As used herein, “tryptase beta” refers to any native tryptase beta from any vertebrate source, including mammals such as primates (e.g., humans) and rodents (e.g., mice and rats), unless otherwise indicated. Tryptase beta is a serine protease that is a major constituent of mast cell secretory granules. As used herein, the term encompasses tryptase beta 1 (encoded by the TPSAB1 gene, which also encodes tryptase alpha 1), tryptase beta 2 (encoded by the TPSB2 gene), and tryptase beta 3 (also encoded by the TPSB2 gene). An exemplary human tryptase beta 1 sequence is shown in SEQ ID NO: 12 (see also GenBank Accession No. NP_003285.2). An exemplary human tryptase beta 2 sequence is shown in SEQ ID NO: 13 (see also GenBank Accession No. AAD13876.1). An exemplary human tryptase beta 3 sequence is shown in SEQ ID NO: 14 (see also GenBank Accession No. NP_077078.5). The term tryptase beta encompasses “full-length,” unprocessed tryptase beta as well as tryptase beta that results from post-translational modifications, including proteolytic processing. Full-length, pro-tryptase beta is thought to be processed in two proteolytic steps. First, autocatalytic intermolecular cleavage at R⁻³ occurs, particularly at acidic pH and in the presence of a polyanion (e.g., heparin or dextran sulfate). Next, the remaining pro′ dipeptide is removed (likely by dipeptidyl peptidase I). For full-length human tryptase beta 1, with reference to SEQ ID NO: 12 below, the underlined amino acid residues correspond to the native leader sequence, and the bolded amino acid residues correspond to the pro-domain, which are cleaved to form the mature protein (see, e.g., Sakai et al. J. Clin. Invest. 97:988-995, 1996)

(SEQ ID NO: 12)
MLNLLLLALPVLASRAYAAPAPGQALQRVGIVGGQEAPRSKWPWQVSLR
VHGPYWMHFCGGSLIHPQWVLTAAHCVGPDVKDLAALRVQLREQHLYYQ
DQLLPVSRIIVHPQFYTAQIGADIALLELEEPVNVSSHVHTVTLPPASE
TFPPGMPCWVTGWGDVDNDERLPPPFPLKQVKVPIMENHICDAKYHLGA
YTGDDVRIVRDDMLCAGNTRRDSCQGDSGGPLVCKVNGTWLQAGVVSWG
EGCAQPNRPGIYTRVTYYLDWIHHYVPKKP.

Mature, enzymatically active tryptase beta is typically a homotetramer or heterotetramer, although active monomer has been reported (see, e.g., Fukuoka et al. J. Immunol. 176:3165, 2006). The subunits of the tryptase beta tetramer are held together by hydrophobic and polar interactions between subunits and stabilized by polyanions (particularly heparin and dextran sulfate). The term tryptase can refer to tryptase tetramer or tryptase monomer. Exemplary sequences for mature human tryptase beta 1, beta 2, and beta 3 are shown in SEQ ID NO: 15, SEQ ID NO: 16, and SEQ ID NO: 17, respectively. The active site of each subunit faces into a central pore of the tetramer, which measures approximately 50×30 angstroms (see, e.g., Pereira et al. Nature 392:306-311, 1998). The size of the central pore typically restricts access of the active sites by inhibitors. Exemplary substrates of tryptase beta include, but are not limited to, PAR2, C3, fibrinogen, fibronectin, and kininogen.

A “disorder” or “disease” is any condition that would benefit from treatment with a method of the invention. This includes chronic and acute disorders or diseases including those pathological conditions which predispose the mammal to the disorder in question. Examples of disorders to be treated herein include CSU (e.g., CSU refractory to antihistamines (e.g., sgH1-AHs)).

The term “administering” means the administration of a composition to a patient (e.g., a patient having CSU (e.g., CSU refractory to antihistamines (e.g., sgH1-AHs))). The compositions (e.g., anti-tryptase antibodies) utilized in the methods and uses described herein can be administered, for example, parenterally, intraperitoneally, intramuscularly, intravenously, intradermally, percutaneously, intraarterially, intralesionally, intracranially, intraarticularly, intraprostatically, intrapleurally, intratracheally, intrathecally, intranasally, intravaginally, intrarectally, topically, intratumorally, peritoneally, subcutaneously (e.g., by a pump (e.g., by a patch pump), subconjunctivally, intravesicularly, mucosally, intrapericardially, intraumbilically, intraocularly, intraorbitally, orally, topically, transdermally, intravitreally, periocularly, conjunctivally, subtenonly, intracamerally, subretinally, retrobulbarly, intracanalicularly, by inhalation, by injection, by implantation, by infusion, by continuous infusion, by localized perfusion bathing target cells directly, by catheter, by lavage, in cremes, or in lipid compositions. Parenteral administration includes intramuscular, intravenous, intraarterial, intraperitoneal, or subcutaneous administration. In some instances, administering is performed intravenously. In other instances, administering is performed subcutaneously. The compositions utilized in the methods described herein can also be administered systemically or locally. The method of administration can vary depending on various factors (e.g., the compound or composition being administered and the severity of the condition, disease, or disorder being treated).

The terms “therapeutic agent” or “agent” refer to any agent that is used to treat a disease, e.g., CSU (e.g., CSU refractory to antihistamines (e.g., sgH1-AHs)). A therapeutic agent may be, for example, a polypeptide(s) (e.g., an antibody (e.g., an anti-tryptase antibody (e.g., an anti-tryptase beta antibody)), an immunoadhesin, or a peptibody), an aptamer, a small molecule that can bind to a protein, or a nucleic acid molecule that can bind to a nucleic acid molecule encoding a target (e.g., siRNA), and the like.

The terms “anti-tryptase antibody,” an “antibody that binds to tryptase,” and “antibody that specifically binds tryptase” refer to an antibody that is capable of binding tryptase with sufficient affinity such that the antibody is useful as a diagnostic and/or therapeutic agent in targeting tryptase. In one aspect, the extent of binding of an anti-tryptase antibody to an unrelated, non-tryptase protein is less than about 10% of the binding of the antibody to tryptase as measured, e.g., by a radioimmunoassay (RIA). In certain aspects, an antibody that binds to tryptase has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain aspects, an anti-tryptase antibody binds to an epitope of tryptase that is conserved among tryptase from different species. Exemplary anti-tryptase antibodies are described herein, in U.S. Pat. Nos. 10,738,131 and 10,752,703; in U.S. Patent Application Publication No. US 2018/0230233; and in International Patent Application Publication No. WO 2018/148585, each of which is incorporated herein by reference in its entirety.

A “mast cell” is a type of granulocyte immune cell. Mast cells are typically present in mucosal and epithelial tissues throughout the body. Mast cells contain cytoplasmic granules that store inflammatory mediators, including tryptase (particularly tryptase beta), histamine, heparin, and cytokines. Mast cells can be activated by antigen/IgE/FcεRI cross-linking, which can result in degranulation and release of inflammatory mediators. A mast cell may be a mucosal mast cell or a connective tissue mast cell. See, e.g., Krystel-Whittemore et al. Front. Immunol. 6:620, 2015.

The terms “patient,” “subject,” and “individual,” as used interchangeably herein, refer to any single animal, more specifically a mammal (including such non-human animals as, for example, cats, dogs, horses, rabbits, cows, pigs, sheep, zoo animals, and non-human primates) for which treatment is desired. Even more specifically, the patient herein is a human.

The term “effective amount” refers to an amount of a drug or therapeutic agent (e.g., an anti-tryptase antibody) effective to treat a disease or disorder (e.g., CSU (e.g., CSU refractory to antihistamines (e.g., sgH1-AHs))) in a subject or patient, such as a mammal, e.g., a human.

As used herein, “therapy” or “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. Those in need of treatment include can include those already with the disorder as well as those at risk to have the disorder or those in whom the disorder is to be prevented. A patient may be successfully “treated” for CSU if, for example, after receiving an CSU therapy, the patient shows observable and/or measurable reduction in or absence of one or more of the following: wheals (hives) or itch (pruritis), e.g., as assessed by a reduction in Urticaria Activity Score (UAS) or UAS7, e.g., a reduction from baseline in the patient's UAS7 score, e.g., at Week 4, Week 8, and/or Week 12 after initiation of therapy.

A “response” of a patient or a patient's “responsiveness” to treatment or therapy, for example a therapy including an anti-tryptase antibody, refers to the clinical or therapeutic benefit imparted to a patient at risk for or having CSU (e.g., CSU refractory to antihistamines (e.g., sgH1-AHs)) from or as a result of the treatment. A skilled person will readily be in position to determine whether a patient is responsive. For example, a patient having CSU who is responsive to a therapy including an anti-tryptase antibody may show observable and/or measurable reduction in or absence of one or more CSU symptoms, for example, wheals (hives), itch (pruritis), or angioedema. In some examples, response may be assessed using any appropriate criteria, e.g., the patient's UAS or UAS7 score, Itch-Severity Score (ISS) or ISS7, Hives-Severity Score (HSS) or HSS7, Urticaria Control Test (UCT), Angioedema Activity Score (AAS), Angioedema Control Test (AECT), or other criteria known in the art.

The terms “Chronic Urticaria Index (CU Index®)” and “basophil histamine release assay” refer interchangeably to an assay used to identify whether a patient has elevated levels of serum factors leading to basophil degranulation and increased mediator release. The CU Index® is an in vitro basophil histamine release assay in which patient serum is mixed with donor basophils and the released histamine levels are measured by an immunoassay. See, e.g., Cho et al. Ann. Allergy Asthma Immunol. 110:29-33, 2013, Biagtan et al. J. Allergy Clin. Immunol. 127:1626-1627, 2011, and U.S. Pat. No. 7,824,877, each of which is incorporated herein by reference in its entirety. Specifically, healthy donor blood basophils are incubated with patient serum, a negative control, and a positive control. The cells are centrifuged and the supernatant is recovered for assay of histamine released. Using a quantitative enzyme immunoassay, the histamine released into the supernatant is measured and compared with the total histamine in the basophils. The value used to define CU Index® positivity has been established as an assay threshold based on a healthy control reference range. In some examples, a CU Index® score of ≥10 indicates the patient is CU Index®-positive. The CU Index® is commercially available from Eurofins Viracor. In some examples, a patient with an elevated CU Index® value (i.e., CU Index® positive) may identify that the patient has a more severe and/or refractory CSU. In some examples, an elevated CU Index® value may indicate that the patient has either an autoimmune basis for their urticaria (antibodies for either IgE, FcεRI, or anti-FcεRII) or an alternate histamine releasing factor (see, e.g., Cho et al. supra).

The term “Urticaria Activity Score” and “UAS” refers to a questionnaire used to evaluate CSU symptoms. The UAS is a composite score with numeric severity intensity ratings (0=none to 3=intense/severe) for (a) the number of wheals (hives) and (b) the intensity of the pruritus (itch) over the past 12 hours (twice daily). With respect to hive severity, 0 indicates no hives; 1 indicates between 1 and 6 hives; 2 indicates between 7 and 12 hives, and 3 indicates greater than 12 hives. With respect to itch severity, 0 indicates none; 1 indicates mild; 2 indicates moderate; and 3 indicates severe. In some examples, a “daily UAS” is calculated as the average of the morning and evening UAS scores.

The term “Urticaria Activity Score summed over 7 days” and “UAS7” refers to the daily UAS score summed over 7 days. The maximum UAS7 value is 42. In some examples, a patient having moderate activity urticaria may have a UAS7 value of 16-27. In some examples, a patient having severe activity urticaria may have a UAS7 value of 28-42. In some examples, a patient having well-controlled urticaria may have a UAS7 value of ≤7, e.g., at Week 4, Week 8, and/or Week 12 after initiation of therapy. In some examples, a patient having a complete response may have a UAS7 value of 0, e.g., at Week 4, Week 8, and/or Week 12 after initiation of therapy. In some examples, a patient may achieve the minimally important difference (MID) in UAS7 as a reduction from baseline of ≥11 points, e.g., by Week 12 after initiation of therapy.

The term “second-generation H1 antihistamines” and “sgH1-AHs” refers to a class of histamine H1-receptor antagonists. sgH1-AHs are typically more selective for peripheral H1 receptors relative to central nervous system H1 receptors compared to first-generation H1 antihistamines, which typically reduces the occurrence of adverse drug reactions (e.g., sedation) while still providing effective relief of allergic reactions. Exemplary sgH1-AHs include, but are not limited to, cetirizine, loratadine, ketotifen, rupatadine, bilastine, terfenadine, astemizole, mizolastine, acrivastine, ebastine, bepotastine, quifenadine, azelastine, levocabastine, olopatadine, levocetirizine, desloratadine, and fexofenadine.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “affinity-matured” antibody is one with one or more alterations in one or more HVRs and/or framework regions which result in an improvement in the affinity of the antibody for antigen, compared to a parent antibody which does not possess those alteration(s). Preferred affinity-matured antibodies will have nanomolar or even picomolar affinities for the target antigen. Affinity-matured antibodies are produced by procedures known in the art. For example, Marks et al. Bio/Technology 10:779-783, 1992 describes affinity maturation by VH and VL domain shuffling. Random mutagenesis of HVR and/or framework residues is described by: Barbas et al. Proc. Natl. Acad. Sci. USA 91:3809-3813, 1994; Schier et al. Gene 169:147-155, 1995; Yelton et al. J. Immunol. 155:1994-2004, 1995; Jackson et al. J. Immunol. 154(7):3310-3319, 1995; and Hawkins et al. J. Mol. Biol. 226:889-896, 1992.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary aspects for measuring binding affinity are described in the following.

An “antibody that binds to the same epitope” as a reference antibody refers to an antibody that contacts an overlapping set of amino acid residues of the antigen as compared to the reference antibody or blocks binding of the reference antibody to its antigen in a competition assay by 50% or more, 60% or more, 70% or more, 80% or more, or 90% or more. In some aspects, the set of amino acid residues contacted by the antibody may be completely overlapping or partially overlapping with the set of amino acid residues contacted by the reference antibody. In some aspects, an antibody that binds to the same epitope as a reference antibody blocks binding of the reference antibody to its antigen in a competition assay by 50% or more, 60% or more, 70% or more, 80% or more, or 90% or more, and conversely, the reference antibody blocks binding of the antibody to its antigen in a competition assay by 50% or more, 60% or more, 70% or more, 80% or more, or 90% or more. An exemplary competition assay is provided herein.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870, Example 2; Zapata et al. Protein Eng. 8(10):1057-1062, 1995); single-chain antibody molecules; and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (C_{H}1). Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having an additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al. Sequences of Proteins of Immunological Interest, 5th Ed. Public Health Service, National Institutes of Health, Bethesda, M D, 1991.

“Fv” consists of a dimer of one heavy- and one light-chain variable region domain in tight, noncovalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Pluckthün in The Pharmacology of Monoclonal Antibodies, vol. 113, Rosenburg and Moore eds., Springer-Verlag, New York, pp. 269-315, 1994.

The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al. Proc. Natl. Acad. Sci. USA 90:6444-6448, 1993.

A “blocking” antibody or an “antagonist” antibody is one which inhibits or reduces biological activity of the antigen it binds. Certain blocking antibodies or antagonist antibodies substantially or completely inhibit the biological activity of the antigen. For example, with respect to anti-tryptase antibodies, in some aspects, the activity may be a tryptase enzymatic activity, e.g., protease activity. In other instances, the activity may be tryptase-mediated stimulation of bronchial smooth muscle cell proliferation and/or collagen-based contraction. In other instances, the activity may be mast cell histamine release (e.g., IgE-triggered histamine release and/or tryptase-triggered histamine release). In some aspects, an antibody can inhibit a biological activity of the antigen it binds by at least about 1%, about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100%.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch et al. Annu. Rev. Immunol. 9:457-492, 1991. To assess ADCC activity of a molecule of interest, an in vitro ADCC assay, such as that described in U.S. Pat. No. 5,500,362 or 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in an animal model such as that disclosed in Clynes et al. Proc. Natl. Acad. Sci. USA 95:652-656, 1998.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234, 1997). FcRs are reviewed, for example, in Ravetch et al. Annu. Rev. Immunol. 9:457-492, 1991; Capel et al. Immunomethods 4:25-34, 1994; and de Haas et al. J. Lab. Clin. Med. 126:330-41, 1995. Other FcRs, including those to be identified in the future, are encompassed by the term “FcR” herein. The term also includes the neonatal receptor, FcRn, which is responsible for the transfer of maternal IgGs to the fetus (see, e.g., Guyer et al. J. Immunol. 117:587, 1976; and Kim et al. J. Immunol. 24:249, 1994).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells, and neutrophils; with PBMCs and NK cells being preferred. The effector cells can be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al. J. Immunol. Methods 202:163, 1996, can be performed.

An “epitope” is the portion of the antigen to which the antibody selectively binds. For a polypeptide antigen, a linear epitope can be a peptide portion of about 4-15 (e.g., 4, 5, 6, 7, 8, 9, 10, 11, 12, amino acid residues. A non-linear, conformational epitope may comprise residues of a polypeptide sequence brought to close vicinity in the three-dimensional (3D) structure of the protein. In some aspects, the epitope comprises amino acids that are within 4 angstroms (Å) of any atom of an antibody. In certain aspects, the epitope comprises amino acids that are within 3.5 Å, 3 Å, 2.5 Å, or 2 Å of any atom of an antibody. The amino acid residues of an antibody that contact an antigen (i.e., paratope) can be determined, for example, by determining the crystal structure of the antibody in complex with the antigen or by performing hydrogen/deuterium exchange.

The terms “full-length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human and/or has been made using any of the techniques for making human antibodies. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. Sequences of Proteins of Immunological Interest, Fifth Edition, NIH Publication 91-3242, Bethesda MD, vols. 1-3, 1991. In one aspect, for the VL, the subgroup is subgroup kappa Ill or kappa IV as in Kabat et al. supra. In one aspect, for the VH, the subgroup is subgroup III as in Kabat et al. supra.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some aspects, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the HVRs (e.g., CDRs) correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al. Nature 321:522-525, 1986; Riechmann et al. Nature 332:323-329, 1988; and Presta, Curr. Op. Struct. Biol. 2:593-596, 1992.

The term “hypervariable region” or “HVR” as used herein refers to each of the regions of an antibody variable domain which are hypervariable in sequence (“complementarity determining regions” or “CDRs”). Generally, antibodies comprise six CDRs: three in the VH (CDR-H1, CDR-H2, CDR-H3), and three in the VL (CDR-L1, CDR-L2, CDR-L3). Exemplary CDRs herein include:
(a) CDRs occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917, 1987);
(b) CDRs occurring at amino acid residues 24-34 (L1), 50-56 (L2), 89-97 (L3), 31-35b (H1), 50-65 (H2), and 95-102 (H3) (Kabat et al. Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745, 1996).

Unless otherwise indicated, HVR residues and other residues in the variable domain (e.g., FR residues) are numbered herein according to Kabat et al. supra.

An “immunoconjugate” is an antibody conjugated to one or more heterologous molecule(s), including but not limited to a cytotoxic agent.

The term “isolated” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some aspects, an antibody is purified to greater than 95% or 99% purity as determined by, for example, electrophoretic (e.g., sodium dodecyl sulfate polyacrylamide gel electrophoresis (SDS-PAGE), isoelectric focusing (IEF), capillary electrophoresis) or chromatographic (e.g., ion exchange or reverse phase HPLC) methods. For a review of methods for assessment of antibody purity, see, for example, Flatman et al. J. Chromatogr. B 848:79-87, 2007. In preferred aspects, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

The term “monoclonal antibody” as used herein refers to an antibody obtained from a population of substantially homogeneous antibodies, i.e., the individual antibodies comprising the population are identical and/or bind the same epitope on an antigen, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In certain aspects, the term “monoclonal antibody” encompasses bispecific antibodies.

The term “bivalent antibody” refers to an antibody that has two binding sites for the antigen. A bivalent antibody can be, without limitation, in the IgG format or in the F(ab′)2 format.

The term “multispecific antibody” is used in the broadest sense and covers an antibody that binds to two or more determinants or epitopes on one antigen or two or more determinants or epitopes on more than one antigen. Such multispecific antibodies include, but are not limited to, full-length antibodies, antibodies having two or more VL and VH domains, antibody fragments such as Fab, Fv, dsFv, scFv, diabodies, bispecific diabodies and triabodies, antibody fragments that have been linked covalently or non-covalently. “Polyepitopic specificity” refers to the ability to specifically bind to two or more different epitopes on the same or different target(s). In certain aspects, the multispecific antibody is a bispecific antibody. “Dual specificity” or “bispecificity” refers to the ability to specifically bind to two different epitopes on the same or different target(s). However, in contrast to bispecific antibodies, dual-specific antibodies have two antigen-binding arms that are identical in amino acid sequence and each Fab arm is capable of recognizing two antigens. Dual-specificity allows the antibodies to interact with high affinity with two different antigens as a single Fab or IgG molecule. According to one aspect, the multispecific antibody binds to each epitope with an affinity of 5 μM to 0.001 pM, 3 μM to 0.001 pM, 1 μM to 0.001 pM, 0.5 μM to 0.001 pM or 0.1 μM to 0.001 pM. “Monospecific” refers to the ability to bind only one epitope.

A “naked antibody” refers to an antibody that is not conjugated to a heterologous moiety (e.g., a cytotoxic moiety) or radiolabel. The naked antibody may be present in a pharmaceutical composition.

With regard to the binding of an antibody to a target molecule, the term “binds” or “binding” or “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a K_{D} for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a K_{D} in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and K_{D} values are inversely related. A high affinity for an antigen is measured by a low K_{D} value. In one aspect, the term “specific binding” refers to binding where a molecule binds to a particular polypeptide or epitope on a particular polypeptide without substantially binding to any other polypeptide or polypeptide epitope.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al. supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al. supra). The “EU numbering system” or “EU index” is generally used when referring to a residue in an immunoglobulin heavy chain constant region (e.g., the EU index reported in Kabat et al. supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see U.S. Provisional Application No. 60/640,323, Figures for EU numbering).

“Percent (%) amino acid sequence identity” with respect to the polypeptide sequences identified herein is defined as the percentage of amino acid residues in a candidate sequence that are identical with the amino acid residues in the polypeptide being compared, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, California. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “package insert” is used to refer to instructions customarily included in commercial packages of therapeutic products, that contain information about the indications, usage, dosage, administration, combination therapy, contraindications and/or warnings concerning the use of such therapeutic products.

The terms “pharmaceutical formulation” and “pharmaceutical composition” are used interchangeably herein, and refer to a preparation which is in such form as to permit the biological activity of an active ingredient contained therein to be effective, and which contains no additional components which are unacceptably toxic to a subject to which the formulation would be administered. Such formulations are sterile.

A “sterile” pharmaceutical formulation is aseptic or free or essentially free from all living microorganisms and their spores.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

A “kit” is any manufacture (e.g., a package or container) comprising at least one reagent, for example, a medicament for treatment of CSU (e.g., CSU refractory to antihistamines (e.g., sgH1-AHs)) (e.g., an anti-tryptase antibody). The manufacture is preferably promoted, distributed, or sold as a unit for performing the methods of the present disclosure.

### III. Therapeutic Methods, Compositions for Use, and Uses of the Invention

The present invention features methods of treating a patient having CSU (e.g., CSU refractory to antihistamines (e.g., sgH1-AHs)), compositions (e.g., anti-tryptase antibodies) for use in treating a patient having CSU, and uses of an anti-tryptase antibody, e.g., in the manufacture or preparation of a medicament for treating a patient having CSU.

In one aspect, provided herein is a method of treating a patient having CSU (e.g., CSU refractory to antihistamines (e.g., sgH1-AHs)), the method comprising administering to a patient having CSU an anti-tryptase beta antibody in a dosing regimen comprising a dosing cycle, wherein the dosing cycle comprises between two and twelve doses, wherein a total of about 150 mg to about 43,200 mg of the anti-tryptase antibody is administered SC or IV to the patient in the dosing cycle.

In another aspect, provided herein is an anti-tryptase beta antibody for use in treating a patient having CSU (e.g., CSU refractory to antihistamines (e.g., sgH1-AHs)), wherein the anti-tryptase beta antibody is for administration to a patient having CSU in a dosing regimen comprising a dosing cycle, wherein the dosing cycle comprises between two and twelve doses, wherein a total of about 150 mg to about 43,200 mg of the anti-tryptase antibody is administered SC or IV to the patient in the dosing cycle.

In yet another aspect, provided herein is the use of an anti-tryptase beta antibody in the manufacture of a medicament for treating a patient having CSU (e.g., CSU refractory to antihistamines (e.g., sgH1-AHs)), wherein the medicament is for administration to a patient having CSU in a dosing regimen comprising a dosing cycle, wherein the dosing cycle comprises between two and twelve doses, wherein a total of about 150 mg to about 43,200 mg of the anti-tryptase antibody is administered SC or IV to the patient in the dosing cycle.

In one aspect, provided herein is a method of treating a patient having CSU (e.g., CSU refractory to antihistamines (e.g., sgH1-AHs)), the method comprising administering to a patient having CSU an anti-tryptase beta antibody in a dosing regimen comprising a dosing cycle, wherein the dosing cycle comprises between two and six doses, wherein a total of about 300 mg to about 21,600 mg of the anti-tryptase antibody is administered SC or IV to the patient in the dosing cycle.

In another aspect, provided herein is an anti-tryptase beta antibody for use in treating a patient having CSU (e.g., CSU refractory to antihistamines (e.g., sgH1-AHs)), wherein the anti-tryptase beta antibody is for administration to a patient having CSU in a dosing regimen comprising a dosing cycle, wherein the dosing cycle comprises between two and six doses, wherein a total of about 300 mg to about 21,600 mg of the anti-tryptase antibody is administered SC or IV to the patient in the dosing cycle.

In yet another aspect, provided herein is the use of an anti-tryptase beta antibody in the manufacture of a medicament for treating a patient having CSU (e.g., CSU refractory to antihistamines (e.g., sgH1-AHs)), wherein the medicament is for administration to a patient having CSU in a dosing regimen comprising a dosing cycle, wherein the dosing cycle comprises between two and six doses, wherein a total of about 300 mg to about 21,600 mg of the anti-tryptase antibody is administered SC or IV to the patient in the dosing cycle.

For example, in some aspects, the dosing cycle comprises two, three, four, five, or six doses. In some aspects, the dosing cycle comprises two doses. In other aspects, the dosing cycle comprises three doses. In other aspects, the dosing cycle comprises four doses. In other aspects, the dosing cycle comprises five doses. In other aspects, the dosing cycle comprises six doses.

In some aspects, a total of about 300 mg to about 21,600 mg, about 300 mg to about 21,000 mg, about 300 mg to about 20,000 mg, about 300 mg to about 19,000 mg, about 300 mg to about 18,000 mg, about 300 mg to about 17,000 mg, about 300 mg to about 16,000 mg, about 300 mg to about 15,000 mg, about 300 mg to about 14,000 mg, about 300 mg to about 13,000 mg, about 300 mg to about 12,000 mg, about 300 mg to about 11,000 mg, about 300 mg to about 10,000 mg, about 300 mg to about 9,000 mg, about 300 mg to about 8,000 mg, about 300 mg to about 7,000 mg, about 300 mg to about 6,000 mg, about 300 mg to about 7,000 mg, about 300 mg to about 6,000 mg, about 300 mg to about 5,000 mg, about 300 mg to about 4,000 mg, about 300 mg to about 3,000 mg, about 300 mg to about 2,000 mg, about 300 mg to about 1,000 mg, about 300 mg to about 900 mg, about 300 mg to about 600 mg, about 600 mg to about 21,600 mg, about 600 mg to about 21,000 mg, about 600 mg to about 20,000 mg, about 600 mg to about 19,000 mg, about 600 mg to about 18,000 mg, about 600 mg to about 17,000 mg, about 600 mg to about 16,000 mg, about 600 mg to about 15,000 mg, about 600 mg to about 14,000 mg, about 600 mg to about 13,000 mg, about 600 mg to about 12,000 mg, about 600 mg to about 11,000 mg, about 600 mg to about 10,000 mg, about 600 mg to about 9,000 mg, about 600 mg to about 8,000 mg, about 600 mg to about 7,000 mg, about 600 mg to about 6,000 mg, about 600 mg to about 7,000 mg, about 600 mg to about 6,000 mg, about 600 mg to about 5,000 mg, about 600 mg to about 4,000 mg, about 600 mg to about 3,000 mg, about 600 mg to about 2,000 mg, about 600 mg to about 1,000 mg, about 600 mg to about 900 mg, about 900 mg to about 21,600 mg, about 900 mg to about 21,000 mg, about 900 mg to about 20,000 mg, about 900 mg to about 19,000 mg, about 900 mg to about 18,000 mg, about 900 mg to about 17,000 mg, about 900 mg to about 16,000 mg, about 900 mg to about 15,000 mg, about 900 mg to about 14,000 mg, about 900 mg to about 13,000 mg, about 900 mg to about 12,000 mg, about 900 mg to about 11,000 mg, about 900 mg to about 10,000 mg, about 900 mg to about 9,000 mg, about 900 mg to about 8,000 mg, about 900 mg to about 7,000 mg, about 900 mg to about 6,000 mg, about 900 mg to about 7,000 mg, about 900 mg to about 6,000 mg, about 900 mg to about 5,000 mg, about 900 mg to about 4,000 mg, about 900 mg to about 3,000 mg, about 900 mg to about 2,000 mg, about 900 mg to about 1,000 mg, about 1,000 mg to about 21,600 mg, about 1,000 mg to about 21,000 mg, about 1,000 mg to about 20,000 mg, about 1,000 mg to about 19,000 mg, about 1,000 mg to about 18,000 mg, about 1,000 mg to about 17,000 mg, about 1,000 mg to about 16,000 mg, about 1,000 mg to about 15,000 mg, about 1,000 mg to about 14,000 mg, about 1,000 mg to about 13,000 mg, about 1,000 mg to about 12,000 mg, about 1,000 mg to about 11,000 mg, about 1,000 mg to about 10,000 mg, about 1,000 mg to about 9,000 mg, about 1,000 mg to about 8,000 mg, about 1,000 mg to about 7,000 mg, about 1,000 mg to about 6,000 mg, about 1,000 mg to about 7,000 mg, about 1,000 mg to about 6,000 mg, about 1,000 mg to about 5,000 mg, about 1,000 mg to about 4,000 mg, about 1,000 mg to about 3,000 mg, about 1,000 mg to about 2,000 mg, about 2,000 mg to about 21,600 mg, about 2,000 mg to about 21,000 mg, about 2,000 mg to about 20,000 mg, about 2,000 mg to about 19,000 mg, about 2,000 mg to about 18,000 mg, about 2,000 mg to about 17,000 mg, about 2,000 mg to about 16,000 mg, about 2,000 mg to about 15,000 mg, about 2,000 mg to about 14,000 mg, about 2,000 mg to about 13,000 mg, about 2,000 mg to about 12,000 mg, about 2,000 mg to about 11,000 mg, about 2,000 mg to about 10,000 mg, about 2,000 mg to about 9,000 mg, about 2,000 mg to about 8,000 mg, about 2,000 mg to about 7,000 mg, about 2,000 mg to about 6,000 mg, about 2,000 mg to about 7,000 mg, about 2,000 mg to about 6,000 mg, about 2,000 mg to about 5,000 mg, about 2,000 mg to about 4,000 mg, about 2,000 mg to about 3,000 mg, about 3,000 mg to about 21,600 mg, about 3,000 mg to about 21,000 mg, about 3,000 mg to about 20,000 mg, about 3,000 mg to about 19,000 mg, about 3,000 mg to about 18,000 mg, about 3,000 mg to about 17,000 mg, about 3,000 mg to about 16,000 mg, about 3,000 mg to about 15,000 mg, about 3,000 mg to about 14,000 mg, about 3,000 mg to about 13,000 mg, about 3,000 mg to about 12,000 mg, about 3,000 mg to about 11,000 mg, about 3,000 mg to about 10,000 mg, about 3,000 mg to about 9,000 mg, about 3,000 mg to about 8,000 mg, about 3,000 mg to about 7,000 mg, about 3,000 mg to about 6,000 mg, about 3,000 mg to about 7,000 mg, about 3,000 mg to about 6,000 mg, about 3,000 mg to about 5,000 mg, about 3,000 mg to about 4,000 mg, about 4,000 mg to about 21,600 mg, about 4,000 mg to about 21,000 mg, about 4,000 mg to about 20,000 mg, about 4,000 mg to about 19,000 mg, about 4,000 mg to about 18,000 mg, about 4,000 mg to about 17,000 mg, about 4,000 mg to about 16,000 mg, about 4,000 mg to about 15,000 mg, about 4,000 mg to about 14,000 mg, about 4,000 mg to about 13,000 mg, about 4,000 mg to about 12,000 mg, about 4,000 mg to about 11,000 mg, about 4,000 mg to about 10,000 mg, about 4,000 mg to about 9,000 mg, about 4,000 mg to about 8,000 mg, about 4,000 mg to about 7,000 mg, about 4,000 mg to about 6,000 mg, about 4,000 mg to about 7,000 mg, about 4,000 mg to about 6,000 mg, about 4,000 mg to about 5,000 mg, about 5,000 mg to about 21,600 mg, about 5,000 mg to about 21,000 mg, about 5,000 mg to about 20,000 mg, about 5,000 mg to about 19,000 mg, about 5,000 mg to about 18,000 mg, about 5,000 mg to about 17,000 mg, about 5,000 mg to about 16,000 mg, about 5,000 mg to about 15,000 mg, about 5,000 mg to about 14,000 mg, about 5,000 mg to about 13,000 mg, about 5,000 mg to about 12,000 mg, about 5,000 mg to about 11,000 mg, about 5,000 mg to about 10,000 mg, about 5,000 mg to about 9,000 mg, about 5,000 mg to about 8,000 mg, about 5,000 mg to about 7,000 mg, about 5,000 mg to about 6,000 mg, about 5,000 mg to about 7,000 mg, about 5,000 mg to about 6,000 mg, about 6,000 mg to about 21,600 mg, about 6,000 mg to about 21,000 mg, about 6,000 mg to about 20,000 mg, about 6,000 mg to about 19,000 mg, about 6,000 mg to about 18,000 mg, about 6,000 mg to about 17,000 mg, about 6,000 mg to about 16,000 mg, about 6,000 mg to about 15,000 mg, about 6,000 mg to about 14,000 mg, about 6,000 mg to about 13,000 mg, about 6,000 mg to about 12,000 mg, about 6,000 mg to about 11,000 mg, about 6,000 mg to about 10,000 mg, about 6,000 mg to about 9,000 mg, about 6,000 mg to about 8,000 mg, about 6,000 mg to about 7,000 mg, about 7,000 mg to about 21,600 mg, about 7,000 mg to about 21,000 mg, about 7,000 mg to about 20,000 mg, about 7,000 mg to about 19,000 mg, about 7,000 mg to about 18,000 mg, about 7,000 mg to about 17,000 mg, about 7,000 mg to about 16,000 mg, about 7,000 mg to about 15,000 mg, about 7,000 mg to about 14,000 mg, about 7,000 mg to about 13,000 mg, about 7,000 mg to about 12,000 mg, about 7,000 mg to about 11,000 mg, about 7,000 mg to about 10,000 mg, about 7,000 mg to about 9,000 mg, about 7,000 mg to about 8,000 mg, about 8,000 mg to about 21,600 mg, about 8,000 mg to about 21,000 mg, about 8,000 mg to about 20,000 mg, about 8,000 mg to about 19,000 mg, about 8,000 mg to about 18,000 mg, about 8,000 mg to about 17,000 mg, about 8,000 mg to about 16,000 mg, about 8,000 mg to about 15,000 mg, about 8,000 mg to about 14,000 mg, about 8,000 mg to about 13,000 mg, about 8,000 mg to about 12,000 mg, about 8,000 mg to about 11,000 mg, about 8,000 mg to about 10,000 mg, about 8,000 mg to about 9,000 mg, about 9,000 mg to about 21,600 mg, about 9,000 mg to about 21,000 mg, about 9,000 mg to about 20,000 mg, about 9,000 mg to about 19,000 mg, about 9,000 mg to about 18,000 mg, about 9,000 mg to about 17,000 mg, about 9,000 mg to about 16,000 mg, about 9,000 mg to about 15,000 mg, about 9,000 mg to about 14,000 mg, about 9,000 mg to about 13,000 mg, about 9,000 mg to about 12,000 mg, about 9,000 mg to about 11,000 mg, about 9,000 mg to about 10,000 mg, about 10,000 mg to about 21,600 mg, about 10,000 mg to about 21,000 mg, about 10,000 mg to about 20,000 mg, about 10,000 mg to about 19,000 mg, about 10,000 mg to about 18,000 mg, about 10,000 mg to about 17,000 mg, about 10,000 mg to about 16,000 mg, about 10,000 mg to about 15,000 mg, about 10,000 mg to about 14,000 mg, about 10,000 mg to about 13,000 mg, about 10,000 mg to about 12,000 mg, about 10,000 mg to about 11,000 mg, about 11,000 mg to about 21,600 mg, about 11,000 mg to about 21,000 mg, about 11,000 mg to about 20,000 mg, about 11,000 mg to about 19,000 mg, about 11,000 mg to about 18,000 mg, about 11,000 mg to about 17,000 mg, about 11,000 mg to about 16,000 mg, about 11,000 mg to about 15,000 mg, about 11,000 mg to about 14,000 mg, about 11,000 mg to about 13,000 mg, about 11,000 mg to about 12,000 mg, about 12,000 mg to about 21,600 mg, about 12,000 mg to about 21,000 mg, about 12,000 mg to about 20,000 mg, about 12,000 mg to about 19,000 mg, about 12,000 mg to about 18,000 mg, about 12,000 mg to about 17,000 mg, about 12,000 mg to about 16,000 mg, about 12,000 mg to about 15,000 mg, about 12,000 mg to about 14,000 mg, about 12,000 mg to about 13,000 mg, about 13,000 mg to about 21,600 mg, about 13,000 mg to about 21,000 mg, about 13,000 mg to about 20,000 mg, about 13,000 mg to about 19,000 mg, about 13,000 mg to about 18,000 mg, about 13,000 mg to about 17,000 mg, about 13,000 mg to about 16,000 mg, about 13,000 mg to about 15,000 mg, about 13,000 mg to about 14,000 mg, about 14,000 mg to about 21,600 mg, about 14,000 mg to about 21,000 mg, about 14,000 mg to about 20,000 mg, about 14,000 mg to about 19,000 mg, about 14,000 mg to about 18,000 mg, about 14,000 mg to about 17,000 mg, about 14,000 mg to about 16,000 mg, about 14,000 mg to about 15,000 mg, about 15,000 mg to about 21,600 mg, about 15,000 mg to about 21,000 mg, about 15,000 mg to about 20,000 mg, about 15,000 mg to about 19,000 mg, about 15,000 mg to about 18,000 mg, about 15,000 mg to about 17,000 mg, about 15,000 mg to about 16,000 mg, about 16,000 mg to about 21,600 mg, about 16,000 mg to about 21,000 mg, about 16,000 mg to about 20,000 mg, about 16,000 mg to about 19,000 mg, about 16,000 mg to about 18,000 mg, about 16,000 mg to about 17,000 mg, about 17,000 mg to about 21,600 mg, about 17,000 mg to about 21,000 mg, about 17,000 mg to about 20,000 mg, about 17,000 mg to about 19,000 mg, about 17,000 mg to about 18,000 mg, about 18,000 mg to about 21,600 mg, about 18,000 mg to about 21,000 mg, about 18,000 mg to about 20,000 mg, about 18,000 mg to about 19,000 mg, about 19,000 mg to about 21,600 mg, about 19,000 mg to about 21,000 mg, about 19,000 mg to about 20,000 mg, about 20,000 mg to about 21,600 mg, about 20,000 mg to about 21,000 mg, or about 21,000 mg to about 21,600 mg of the anti-tryptase antibody is administered SC or IV to the patient in the dosing cycle.

The anti-tryptase antibody may be administered to the patient in any suitable dose, e.g., any dose described herein. In some instances, the dose is 300 mg SC, 600 mg SC, 900 mg IV, or 1800 mg IV.

For example, in one aspect, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of from about 50 mg to about 1000 mg SC (e.g., by a pump (e.g., by a patch pump)).

In another aspect, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of from about 50 mg to about 1000 mg SC (e.g., by a pump (e.g., by a patch pump)).

In another aspect, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of from about 50 mg to about 1000 mg SC (e.g., by a pump (e.g., by a patch pump)).

In one aspect, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of from about 450 mg to about 3600 mg IV.

In another aspect, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of from about 450 mg to about 3600 mg IV.

In another aspect, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of from about 450 mg to about 3600 mg IV.

In one aspect, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of from about 300 mg to about 3600 mg. The C1D1 may be administered, for example, intravenously (IV) or subcutaneously (SC). In some aspects, the C1D1 is administered IV. In other aspects, the C1D1 is administered SC (e.g., by a pump (e.g., by a patch pump)).

In another aspect, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of from about 300 mg to about 3600 mg. In some aspects, the C1D1 is administered IV. In other aspects, the C1D1 is administered SC (e.g., by a pump (e.g., by a patch pump)).

In another aspect, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of from about 300 mg to about 3600 mg. In some aspects, the C1D1 is administered IV. In other aspects, the C1D1 is administered SC (e.g., by a pump (e.g., by a patch pump)).

For example, in one aspect, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of from about 25 mg to about 450 mg (e.g., about 300 mg). The C1D1 may be administered, for example, IV or SC. In some aspects, the C1D1 is administered IV. In other aspects, the C1D1 is administered SC (e.g., by a pump (e.g., by a patch pump)).

In another aspect, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of from about 25 mg to about 450 mg (e.g., about 300 mg). In some aspects, the C1D1 is administered IV. In other aspects, the C1D1 is administered SC (e.g., by a pump (e.g., by a patch pump)).

In another aspect, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of from about 25 mg to about 450 mg (e.g., about 300 mg). In some aspects, the C1D1 is administered IV. In other aspects, the C1D1 is administered SC (e.g., by a pump (e.g., by a patch pump)).

For example, in any of the preceding aspects, the first dose (C1D1) of the anti-tryptase antibody, and/or any additional doses of the anti-tryptase antibody, may be about 25 mg to about 450 mg, about 25 mg to about 425 mg, about 25 mg to about 400 mg, about 25 mg to about 375 mg, about 25 mg to about 350 mg, about 25 mg to about 325 mg, about 25 mg to about 300 mg, about 25 mg to about 275 mg, about 25 mg to about 250 mg, about 25 mg to about 225 mg, about 25 mg to about 200 mg, about 25 mg to about 175 mg, about 25 mg to about 150 mg, about 25 mg to about 125 mg, about 25 mg to about 100 mg, about 25 mg to about 75 mg, about 25 mg to about 50 mg, about 50 mg to about 450 mg, about 50 mg to about 425 mg, about 50 mg to about 400 mg, about 50 mg to about 375 mg, about 50 mg to about 350 mg, about 50 mg to about 325 mg, about 50 mg to about 300 mg, about 50 mg to about 275 mg, about 50 mg to about 250 mg, about 50 mg to about 225 mg, about 50 mg to about 200 mg, about 50 mg to about 175 mg, about 50 mg to about 150 mg, about 50 mg to about 125 mg, about 50 mg to about 100 mg, about 50 mg to about 75 mg, about 75 mg to about 450 mg, about 75 mg to about 425 mg, about 75 mg to about 400 mg, about 75 mg to about 375 mg, about 75 mg to about 350 mg, about 75 mg to about 325 mg, about 75 mg to about 300 mg, about 75 mg to about 275 mg, about 75 mg to about 250 mg, about 75 mg to about 225 mg, about 75 mg to about 200 mg, about 75 mg to about 175 mg, about 75 mg to about 150 mg, about 75 mg to about 125 mg, about 75 mg to about 100 mg, about 100 mg to about 450 mg, about 100 mg to about 425 mg, about 100 mg to about 400 mg, about 100 mg to about 375 mg, about 100 mg to about 350 mg, about 100 mg to about 325 mg, about 100 mg to about 300 mg, about 100 mg to about 275 mg, about 100 mg to about 250 mg, about 100 mg to about 225 mg, about 100 mg to about 200 mg, about 100 mg to about 175 mg, about 100 mg to about 150 mg, about 100 mg to about 125 mg, about 125 mg to about 450 mg, about 125 mg to about 425 mg, about 125 mg to about 400 mg, about 125 mg to about 375 mg, about 125 mg to about 350 mg, about 125 mg to about 325 mg, about 125 mg to about 300 mg, about 125 mg to about 275 mg, about 125 mg to about 250 mg, about 125 mg to about 225 mg, about 125 mg to about 200 mg, about 125 mg to about 175 mg, about 125 mg to about 150 mg, about 150 mg to about 450 mg, about 150 mg to about 425 mg, about 150 mg to about 400 mg, about 150 mg to about 375 mg, about 150 mg to about 350 mg, about 150 mg to about 325 mg, about 150 mg to about 300 mg, about 150 mg to about 275 mg, about 150 mg to about 250 mg, about 150 mg to about 225 mg, about 150 mg to about 200 mg, about 150 mg to about 175 mg, about 175 mg to about 450 mg, about 175 mg to about 425 mg, about 175 mg to about 400 mg, about 175 mg to about 375 mg, about 175 mg to about 350 mg, about 175 mg to about 325 mg, about 175 mg to about 300 mg, about 175 mg to about 275 mg, about 175 mg to about 250 mg, about 175 mg to about 225 mg, about 175 mg to about 200 mg, about 200 mg to about 450 mg, about 200 mg to about 425 mg, about 200 mg to about 400 mg, about 200 mg to about 375 mg, about 200 mg to about 350 mg, about 200 mg to about 325 mg, about 200 mg to about 300 mg, about 200 mg to about 275 mg, about 200 mg to about 250 mg, about 200 mg to about 225 mg, about 225 mg to about 450 mg, about 225 mg to about 425 mg, about 225 mg to about 400 mg, about 225 mg to about 375 mg, about 225 mg to about 350 mg, about 225 mg to about 325 mg, about 225 mg to about 300 mg, about 225 mg to about 275 mg, about 225 mg to about 250 mg, about 250 mg to about 450 mg, about 250 mg to about 425 mg, about 250 mg to about 400 mg, about 250 mg to about 375 mg, about 250 mg to about 350 mg, about 250 mg to about 325 mg, about 250 mg to about 300 mg, about 250 mg to about 275 mg, about 275 mg to about 450 mg, about 275 mg to about 425 mg, about 275 mg to about 400 mg, about 275 mg to about 375 mg, about 275 mg to about 350 mg, about 275 mg to about 325 mg, about 275 mg to about 300 mg, about 300 mg to about 450 mg, about 300 mg to about 425 mg, about 300 mg to about 400 mg, about 300 mg to about 375 mg, about 300 mg to about 350 mg, about 300 mg to about 325 mg, about 325 mg to about 450 mg, about 325 mg to about 425 mg, about 325 mg to about 400 mg, about 325 mg to about 375 mg, about 325 mg to about 350 mg, about 350 mg to about 450 mg, about 350 mg to about 425 mg, about 350 mg to about 400 mg, about 350 mg to about 375 mg, about 375 mg to about 450 mg, about 375 mg to about 425 mg, about 375 mg to about 400 mg, about 400 mg to about 450 mg, about 400 mg to about 425 mg, or about 425 mg to about 450 mg.

In one aspect, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of from about 300 mg to about 750 mg (e.g., about 450 mg). The C1D1 may be administered, for example, IV or SC. In some aspects, the C1D1 is administered IV. In other aspects, the C1D1 is administered SC (e.g., by a pump (e.g., by a patch pump)).

In another aspect, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of from about 300 mg to about 750 mg (e.g., about 450 mg). In some aspects, the C1D1 is administered IV. In other aspects, the C1D1 is administered SC (e.g., by a pump (e.g., by a patch pump)).

In another aspect, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of from about 300 mg to about 750 mg (e.g., about 450 mg). In some aspects, the C1D1 is administered IV. In other aspects, the C1D1 is administered SC (e.g., by a pump (e.g., by a patch pump)).

For example, in any of the preceding aspects, the first dose (C1D1) of the anti-tryptase antibody, and/or any additional doses of the anti-tryptase antibody, may be about 300 mg to about 750 mg, about 300 mg to about 725 mg, about 300 mg to about 700 mg, about 300 mg to about 675 mg, about 300 mg to about 650 mg, about 300 mg to about 625 mg, about 300 mg to about 600 mg, about 300 mg to about 575 mg, about 300 mg to about 550 mg, about 300 mg to about 525 mg, about 300 mg to about 500 mg, about 300 mg to about 475 mg, about 300 mg to about 450 mg, about 300 mg to about 425 mg, about 300 mg to about 400 mg, about 300 mg to about 375 mg, about 300 mg to about 350 mg, about 300 mg to about 325 mg, about 325 mg to about 750 mg, about 325 mg to about 725 mg, about 325 mg to about 700 mg, about 325 mg to about 675 mg, about 325 mg to about 650 mg, about 325 mg to about 625 mg, about 325 mg to about 600 mg, about 325 mg to about 575 mg, about 325 mg to about 550 mg, about 325 mg to about 525 mg, about 325 mg to about 500 mg, about 325 mg to about 475 mg, about 325 mg to about 450 mg, about 325 mg to about 425 mg, about 325 mg to about 400 mg, about 325 mg to about 375 mg, about 325 mg to about 350 mg, about 350 mg to about 750 mg, about 350 mg to about 725 mg, about 350 mg to about 700 mg, about 350 mg to about 675 mg, about 350 mg to about 650 mg, about 350 mg to about 625 mg, about 350 mg to about 600 mg, about 350 mg to about 575 mg, about 350 mg to about 550 mg, about 350 mg to about 525 mg, about 350 mg to about 500 mg, about 350 mg to about 475 mg, about 350 mg to about 450 mg, about 350 mg to about 425 mg, about 350 mg to about 400 mg, about 350 mg to about 375 mg, about 375 mg to about 750 mg, about 375 mg to about 725 mg, about 375 mg to about 700 mg, about 375 mg to about 675 mg, about 375 mg to about 650 mg, about 375 mg to about 625 mg, about 375 mg to about 600 mg, about 375 mg to about 575 mg, about 375 mg to about 550 mg, about 375 mg to about 525 mg, about 375 mg to about 500 mg, about 375 mg to about 475 mg, about 375 mg to about 450 mg, about 375 mg to about 425 mg, about 375 mg to about 400 mg, about 400 mg to about 750 mg, about 400 mg to about 725 mg, about 400 mg to about 700 mg, about 400 mg to about 675 mg, about 400 mg to about 650 mg, about 400 mg to about 625 mg, about 400 mg to about 600 mg, about 400 mg to about 575 mg, about 400 mg to about 550 mg, about 400 mg to about 525 mg, about 400 mg to about 500 mg, about 400 mg to about 475 mg, about 400 mg to about 450 mg, about 400 mg to about 425 mg, about 425 mg to about 750 mg, about 425 mg to about 725 mg, about 425 mg to about 700 mg, about 425 mg to about 675 mg, about 425 mg to about 650 mg, about 425 mg to about 625 mg, about 425 mg to about 600 mg, about 425 mg to about 575 mg, about 425 mg to about 550 mg, about 425 mg to about 525 mg, about 425 mg to about 500 mg, about 425 mg to about 475 mg, about 425 mg to about 450 mg, about 450 mg to about 750 mg, about 450 mg to about 725 mg, about 450 mg to about 700 mg, about 450 mg to about 675 mg, about 450 mg to about 650 mg, about 450 mg to about 625 mg, about 450 mg to about 600 mg, about 450 mg to about 575 mg, about 450 mg to about 550 mg, about 450 mg to about 525 mg, about 450 mg to about 500 mg, about 450 mg to about 475 mg, about 475 mg to about 750 mg, about 475 mg to about 725 mg, about 475 mg to about 700 mg, about 475 mg to about 675 mg, about 475 mg to about 650 mg, about 475 mg to about 625 mg, about 475 mg to about 600 mg, about 475 mg to about 575 mg, about 475 mg to about 550 mg, about 475 mg to about 525 mg, about 475 mg to about 500 mg, about 500 mg to about 750 mg, about 500 mg to about 725 mg, about 500 mg to about 700 mg, about 500 mg to about 675 mg, about 500 mg to about 650 mg, about 500 mg to about 625 mg, about 500 mg to about 600 mg, about 500 mg to about 575 mg, about 500 mg to about 550 mg, about 500 mg to about 525 mg, about 525 mg to about 750 mg, about 525 mg to about 725 mg, about 525 mg to about 700 mg, about 525 mg to about 675 mg, about 525 mg to about 650 mg, about 525 mg to about 625 mg, about 525 mg to about 600 mg, about 525 mg to about 575 mg, about 525 mg to about 550 mg, about 550 mg to about 750 mg, about 550 mg to about 725 mg, about 550 mg to about 700 mg, about 550 mg to about 675 mg, about 550 mg to about 650 mg, about 550 mg to about 625 mg, about 550 mg to about 600 mg, about 550 mg to about 575 mg, about 575 mg to about 750 mg, about 575 mg to about 725 mg, about 575 mg to about 700 mg, about 575 mg to about 675 mg, about 575 mg to about 650 mg, about 575 mg to about 625 mg, about 575 mg to about 600 mg, about 600 mg to about 750 mg, about 600 mg to about 725 mg, about 600 mg to about 700 mg, about 600 mg to about 675 mg, about 600 mg to about 650 mg, about 600 mg to about 625 mg, about 625 mg to about 750 mg, about 625 mg to about 725 mg, about 625 mg to about 700 mg, about 625 mg to about 675 mg, about 625 mg to about 650 mg, about 650 mg to about 750 mg, about 650 mg to about 725 mg, about 650 mg to about 700 mg, about 650 mg to about 675 mg, about 675 mg to about 750 mg, about 675 mg to about 725 mg, about 675 mg to about 700 mg, about 700 mg to about 750 mg, about 700 mg to about 725 mg, or about 725 mg to about 750 mg.

In one aspect, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of from about 400 mg to about 800 mg (e.g., about 600 mg). The C1D1 may be administered, for example, IV or SC. In some aspects, the C1D1 is administered IV. In other aspects, the C1D1 is administered SC (e.g., by a pump (e.g., by a patch pump)).

In another aspect, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of from about 400 mg to about 800 mg (e.g., about 600 mg). In some aspects, the C1D1 is administered IV. In other aspects, the C1D1 is administered SC (e.g., by a pump (e.g., by a patch pump)).

In another aspect, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of from about 400 mg to about 800 mg (e.g., about 600 mg). In some aspects, the C1D1 is administered IV. In other aspects, the C1D1 is administered SC (e.g., by a pump (e.g., by a patch pump)).

For example, in any of the preceding aspects, the first dose (C1D1) of the anti-tryptase antibody, and/or any additional doses of the anti-tryptase antibody, may be about 400 mg to about 800 mg, about 400 mg to about 775 mg, about 400 mg to about 750 mg, about 400 mg to about 725 mg, about 400 mg to about 700 mg, about 400 mg to about 675 mg, about 400 mg to about 650 mg, about 400 mg to about 625 mg, about 400 mg to about 600 mg, about 400 mg to about 575 mg, about 400 mg to about 550 mg, about 400 mg to about 525 mg, about 400 mg to about 500 mg, about 400 mg to about 475 mg, about 400 mg to about 450 mg, about 400 mg to about 425 mg, about 425 mg to about 800 mg, about 425 mg to about 775 mg, about 425 mg to about 750 mg, about 425 mg to about 725 mg, about 425 mg to about 700 mg, about 425 mg to about 675 mg, about 425 mg to about 650 mg, about 425 mg to about 625 mg, about 425 mg to about 600 mg, about 425 mg to about 575 mg, about 425 mg to about 550 mg, about 425 mg to about 525 mg, about 425 mg to about 500 mg, about 425 mg to about 475 mg, about 425 mg to about 450 mg, about 450 mg to about 800 mg, about 450 mg to about 775 mg, about 450 mg to about 750 mg, about 450 mg to about 725 mg, about 450 mg to about 700 mg, about 450 mg to about 675 mg, about 450 mg to about 650 mg, about 450 mg to about 625 mg, about 450 mg to about 600 mg, about 450 mg to about 575 mg, about 450 mg to about 550 mg, about 450 mg to about 525 mg, about 450 mg to about 500 mg, about 450 mg to about 475 mg, about 475 mg to about 800 mg, about 475 mg to about 775 mg, about 475 mg to about 750 mg, about 475 mg to about 725 mg, about 475 mg to about 700 mg, about 475 mg to about 675 mg, about 475 mg to about 650 mg, about 475 mg to about 625 mg, about 475 mg to about 600 mg, about 475 mg to about 575 mg, about 475 mg to about 550 mg, about 475 mg to about 525 mg, about 475 mg to about 500 mg, about 500 mg to about 800 mg, about 500 mg to about 775 mg, about 500 mg to about 750 mg, about 500 mg to about 725 mg, about 500 mg to about 700 mg, about 500 mg to about 675 mg, about 500 mg to about 650 mg, about 500 mg to about 625 mg, about 500 mg to about 600 mg, about 500 mg to about 575 mg, about 500 mg to about 550 mg, about 500 mg to about 525 mg, about 525 mg to about 800 mg, about 525 mg to about 775 mg, about 525 mg to about 750 mg, about 525 mg to about 725 mg, about 525 mg to about 700 mg, about 525 mg to about 675 mg, about 525 mg to about 650 mg, about 525 mg to about 625 mg, about 525 mg to about 600 mg, about 525 mg to about 575 mg, about 525 mg to about 550 mg, about 550 mg to about 800 mg, about 550 mg to about 775 mg, about 550 mg to about 750 mg, about 550 mg to about 725 mg, about 550 mg to about 700 mg, about 550 mg to about 675 mg, about 550 mg to about 650 mg, about 550 mg to about 625 mg, about 550 mg to about 600 mg, about 550 mg to about 575 mg, about 575 mg to about 800 mg, about 575 mg to about 775 mg, about 575 mg to about 750 mg, about 575 mg to about 725 mg, about 575 mg to about 700 mg, about 575 mg to about 675 mg, about 575 mg to about 650 mg, about 575 mg to about 625 mg, about 575 mg to about 600 mg, about 600 mg to about 800 mg, about 600 mg to about 775 mg, about 600 mg to about 750 mg, about 600 mg to about 725 mg, about 600 mg to about 700 mg, about 600 mg to about 675 mg, about 600 mg to about 650 mg, about 600 mg to about 625 mg, about 625 mg to about 800 mg, about 625 mg to about 775 mg, about 625 mg to about 750 mg, about 625 mg to about 725 mg, about 625 mg to about 700 mg, about 625 mg to about 675 mg, about 625 mg to about 650 mg, about 650 mg to about 800 mg, about 650 mg to about 775 mg, about 650 mg to about 750 mg, about 650 mg to about 725 mg, about 650 mg to about 700 mg, about 650 mg to about 675 mg, about 675 mg to about 800 mg, about 675 mg to about 775 mg, about 675 mg to about 750 mg, about 675 mg to about 725 mg, about 675 mg to about 700 mg, about 700 mg to about 800 mg, about 700 mg to about 775 mg, about 700 mg to about 750 mg, about 700 mg to about 725 mg, about 725 mg to about 800 mg, about 725 mg to about 775 mg, about 725 mg to about 750 mg, about 750 mg to about 800 mg, about 750 mg to about 775 mg, or about 775 mg to about 800 mg.

In one aspect, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of from about 450 mg to about 900 mg (e.g., about 750 mg). The C1D1 may be administered, for example, IV or SC. In some aspects, the C1D1 is administered IV. In other aspects, the C1D1 is administered SC (e.g., by a pump (e.g., by a patch pump)).

In another aspect, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of from about 450 mg to about 900 mg (e.g., about 750 mg). In some aspects, the C1D1 is administered IV. In other aspects, the C1D1 is administered SC (e.g., by a pump (e.g., by a patch pump)).

In another aspect, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of from about 450 mg to about 900 mg (e.g., about 750 mg). In some aspects, the C1D1 is administered IV. In other aspects, the C1D1 is administered SC (e.g., by a pump (e.g., by a patch pump)).

For example, in any of the preceding aspects, the first dose (C1D1) of the anti-tryptase antibody, and/or any additional doses of the anti-tryptase antibody, may be about 450 mg to about 900 mg, about 450 mg to about 875 mg, about 450 mg to about 850 mg, about 450 mg to about 825 mg, about 450 mg to about 800 mg, about 450 mg to about 775 mg, about 450 mg to about 750 mg, about 450 mg to about 725 mg, about 450 mg to about 700 mg, about 450 mg to about 675 mg, about 450 mg to about 650 mg, about 450 mg to about 625 mg, about 450 mg to about 600 mg, about 450 mg to about 575 mg, about 450 mg to about 550 mg, about 450 mg to about 525 mg, about 450 mg to about 500 mg, about 450 mg to about 475 mg, about 475 mg to about 900 mg, about 475 mg to about 875 mg, about 475 mg to about 850 mg, about 475 mg to about 825 mg, about 475 mg to about 800 mg, about 475 mg to about 775 mg, about 475 mg to about 750 mg, about 475 mg to about 725 mg, about 475 mg to about 700 mg, about 475 mg to about 675 mg, about 475 mg to about 650 mg, about 475 mg to about 625 mg, about 475 mg to about 600 mg, about 475 mg to about 575 mg, about 475 mg to about 550 mg, about 475 mg to about 525 mg, about 475 mg to about 500 mg, about 500 mg to about 900 mg, about 500 mg to about 875 mg, about 500 mg to about 850 mg, about 500 mg to about 825 mg, about 500 mg to about 800 mg, about 500 mg to about 775 mg, about 500 mg to about 750 mg, about 500 mg to about 725 mg, about 500 mg to about 700 mg, about 500 mg to about 675 mg, about 500 mg to about 650 mg, about 500 mg to about 625 mg, about 500 mg to about 600 mg, about 500 mg to about 575 mg, about 500 mg to about 550 mg, about 500 mg to about 525 mg, about 500 mg to about 900 mg, about 500 mg to about 875 mg, about 500 mg to about 850 mg, about 500 mg to about 825 mg, about 500 mg to about 800 mg, about 500 mg to about 775 mg, about 500 mg to about 750 mg, about 500 mg to about 725 mg, about 500 mg to about 700 mg, about 500 mg to about 675 mg, about 500 mg to about 650 mg, about 500 mg to about 625 mg, about 500 mg to about 600 mg, about 500 mg to about 575 mg, about 500 mg to about 550 mg, about 500 mg to about 525 mg, about 525 mg to about 900 mg, about 525 mg to about 875 mg, about 525 mg to about 850 mg, about 525 mg to about 825 mg, about 525 mg to about 800 mg, about 525 mg to about 775 mg, about 525 mg to about 750 mg, about 525 mg to about 725 mg, about 525 mg to about 700 mg, about 525 mg to about 675 mg, about 525 mg to about 650 mg, about 525 mg to about 625 mg, about 525 mg to about 600 mg, about 525 mg to about 575 mg, about 525 mg to about 550 mg, about 550 mg to about 900 mg, about 550 mg to about 875 mg, about 550 mg to about 850 mg, about 550 mg to about 825 mg, about 550 mg to about 800 mg, about 550 mg to about 775 mg, about 550 mg to about 750 mg, about 550 mg to about 725 mg, about 550 mg to about 700 mg, about 550 mg to about 675 mg, about 550 mg to about 650 mg, about 550 mg to about 625 mg, about 550 mg to about 600 mg, about 550 mg to about 575 mg, about 575 mg to about 900 mg, about 575 mg to about 875 mg, about 575 mg to about 850 mg, about 575 mg to about 825 mg, about 575 mg to about 800 mg, about 575 mg to about 775 mg, about 575 mg to about 750 mg, about 575 mg to about 725 mg, about 575 mg to about 700 mg, about 575 mg to about 675 mg, about 575 mg to about 650 mg, about 575 mg to about 625 mg, about 575 mg to about 600 mg, about 600 mg to about 900 mg, about 600 mg to about 875 mg, about 600 mg to about 850 mg, about 600 mg to about 825 mg, about 600 mg to about 800 mg, about 600 mg to about 775 mg, about 600 mg to about 750 mg, about 600 mg to about 725 mg, about 600 mg to about 700 mg, about 600 mg to about 675 mg, about 600 mg to about 650 mg, about 600 mg to about 625 mg, about 625 mg to about 900 mg, about 625 mg to about 875 mg, about 625 mg to about 850 mg, about 625 mg to about 825 mg, about 625 mg to about 800 mg, about 625 mg to about 775 mg, about 625 mg to about 750 mg, about 625 mg to about 725 mg, about 625 mg to about 700 mg, about 625 mg to about 675 mg, about 625 mg to about 650 mg, about 650 mg to about 900 mg, about 650 mg to about 875 mg, about 650 mg to about 850 mg, about 650 mg to about 825 mg, about 650 mg to about 800 mg, about 650 mg to about 775 mg, about 650 mg to about 750 mg, about 650 mg to about 725 mg, about 650 mg to about 700 mg, about 650 mg to about 675 mg, about 675 mg to about 900 mg, about 675 mg to about 875 mg, about 675 mg to about 850 mg, about 675 mg to about 825 mg, about 675 mg to about 800 mg, about 675 mg to about 775 mg, about 675 mg to about 750 mg, about 675 mg to about 725 mg, about 675 mg to about 700 mg, about 700 mg to about 900 mg, about 700 mg to about 875 mg, about 700 mg to about 850 mg, about 700 mg to about 825 mg, about 700 mg to about 800 mg, about 700 mg to about 775 mg, about 700 mg to about 750 mg, about 700 mg to about 725 mg, about 725 mg to about 900 mg, about 725 mg to about 875 mg, about 725 mg to about 850 mg, about 725 mg to about 825 mg, about 725 mg to about 800 mg, about 725 mg to about 775 mg, about 725 mg to about 750 mg, about 750 mg to about 900 mg, about 750 mg to about 875 mg, about 750 mg to about 850 mg, about 750 mg to about 825 mg, about 750 mg to about 800 mg, about 750 mg to about 775 mg, about 775 mg to about 900 mg, about 775 mg to about 875 mg, about 775 mg to about 850 mg, about 775 mg to about 825 mg, about 775 mg to about 800 mg, about 800 mg to about 900 mg, about 800 mg to about 875 mg, about 800 mg to about 850 mg, about 800 mg to about 825 mg, about 825 mg to about 900 mg, about 825 mg to about 875 mg, about 825 mg to about 850 mg, about 850 mg to about 900 mg, about 850 mg to about 875 mg, or about 875 mg to about 900 mg.

In one aspect, provided herein is a method of treating a patient having CSU (e.g., CSU refractory to antihistamines (e.g., sgH1-AHs)), the method including administering to a patient having CSU an anti-tryptase antibody, wherein the anti-tryptase antibody is for administration to a patient having CSU in a dosing regimen including a dosing cycle, wherein the dosing cycle includes a first dose (C1D1) of the anti-tryptase antibody of from about 750 mg to about 1350 mg (e.g., about 900 mg). The C1D1 may be administered, for example, IV or SC. In some aspects, the C1D1 is administered IV. In other aspects, the C1D1 is administered SC (e.g., by a pump (e.g., by a patch pump)).

In another aspect, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of from about 750 mg to about 1350 mg (e.g., about 900 mg). In some aspects, the C1D1 is administered IV. In other aspects, the C1D1 is administered SC (e.g., by a pump (e.g., by a patch pump)).

In another aspect, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of from about 750 mg to about 1350 mg (e.g., about 900 mg). In some aspects, the C1D1 is administered IV. In other aspects, the C1D1 is administered SC (e.g., by a pump (e.g., by a patch pump)).

For example, in any of the preceding aspects, the first dose (C1D1) of the anti-tryptase antibody, and/or any additional doses of the anti-tryptase antibody, may be about 750 mg to about 1350 mg, about 750 mg to about 1325 mg, about 750 mg to about 1300 mg, about 750 mg to about 1275 mg, about 750 mg to about 1250 mg, about 750 mg to about 1225 mg, about 750 mg to about 1200 mg, about 750 mg to about 1175 mg, about 750 mg to about 1150 mg, about 750 mg to about 1125 mg, about 750 mg to about 1100 mg, about 750 mg to about 1075 mg, about 750 mg to about 1050 mg, about 750 mg to about 1025 mg, about 750 mg to about 1000 mg, about 750 mg to about 975 mg, about 750 mg to about 950 mg, about 750 mg to about 925 mg, about 750 mg to about 900 mg, about 750 mg to about 875 mg, about 750 mg to about 850 mg, about 750 mg to about 825 mg, about 750 mg to about 800 mg, about 750 mg to about 775 mg, about 775 mg to about 1350 mg, about 775 mg to about 1325 mg, about 775 mg to about 1300 mg, about 775 mg to about 1275 mg, about 775 mg to about 1250 mg, about 775 mg to about 1225 mg, about 775 mg to about 1200 mg, about 775 mg to about 1175 mg, about 775 mg to about 1150 mg, about 775 mg to about 1125 mg, about 775 mg to about 1100 mg, about 775 mg to about 1075 mg, about 775 mg to about 1050 mg, about 775 mg to about 1025 mg, about 775 mg to about 1000 mg, about 775 mg to about 975 mg, about 775 mg to about 950 mg, about 775 mg to about 925 mg, about 775 mg to about 900 mg, about 775 mg to about 875 mg, about 775 mg to about 850 mg, about 775 mg to about 825 mg, about 775 mg to about 800 mg, about 800 mg to about 1350 mg, about 800 mg to about 1325 mg, about 800 mg to about 1300 mg, about 800 mg to about 1275 mg, about 800 mg to about 1250 mg, about 800 mg to about 1225 mg, about 800 mg to about 1200 mg, about 800 mg to about 1175 mg, about 800 mg to about 1150 mg, about 800 mg to about 1125 mg, about 800 mg to about 1100 mg, about 800 mg to about 1075 mg, about 800 mg to about 1050 mg, about 800 mg to about 1025 mg, about 800 mg to about 1000 mg, about 800 mg to about 975 mg, about 800 mg to about 950 mg, about 800 mg to about 925 mg, about 800 mg to about 900 mg, about 800 mg to about 875 mg, about 800 mg to about 850 mg, about 800 mg to about 825 mg, about 825 mg to about 1350 mg, about 825 mg to about 1325 mg, about 825 mg to about 1300 mg, about 825 mg to about 1275 mg, about 825 mg to about 1250 mg, about 825 mg to about 1225 mg, about 825 mg to about 1200 mg, about 825 mg to about 1175 mg, about 825 mg to about 1150 mg, about 825 mg to about 1125 mg, about 825 mg to about 1100 mg, about 825 mg to about 1075 mg, about 825 mg to about 1050 mg, about 825 mg to about 1025 mg, about 825 mg to about 1000 mg, about 825 mg to about 975 mg, about 825 mg to about 950 mg, about 825 mg to about 925 mg, about 825 mg to about 900 mg, about 825 mg to about 875 mg, about 825 mg to about 850 mg, about 850 mg to about 1350 mg, about 850 mg to about 1325 mg, about 850 mg to about 1300 mg, about 850 mg to about 1275 mg, about 850 mg to about 1250 mg, about 850 mg to about 1225 mg, about 850 mg to about 1200 mg, about 850 mg to about 1175 mg, about 850 mg to about 1150 mg, about 850 mg to about 1125 mg, about 850 mg to about 1100 mg, about 850 mg to about 1075 mg, about 850 mg to about 1050 mg, about 850 mg to about 1025 mg, about 850 mg to about 1000 mg, about 850 mg to about 975 mg, about 850 mg to about 950 mg, about 850 mg to about 925 mg, about 850 mg to about 900 mg, about 850 mg to about 875 mg, about 875 mg to about 1350 mg, about 875 mg to about 1325 mg, about 875 mg to about 1300 mg, about 875 mg to about 1275 mg, about 875 mg to about 1250 mg, about 875 mg to about 1225 mg, about 875 mg to about 1200 mg, about 875 mg to about 1175 mg, about 875 mg to about 1150 mg, about 875 mg to about 1125 mg, about 875 mg to about 1100 mg, about 875 mg to about 1075 mg, about 875 mg to about 1050 mg, about 875 mg to about 1025 mg, about 875 mg to about 1000 mg, about 875 mg to about 975 mg, about 875 mg to about 950 mg, about 875 mg to about 925 mg, about 875 mg to about 900 mg, about 900 mg to about 1350 mg, about 900 mg to about 1325 mg, about 900 mg to about 1300 mg, about 900 mg to about 1275 mg, about 900 mg to about 1250 mg, about 900 mg to about 1225 mg, about 900 mg to about 1200 mg, about 900 mg to about 1175 mg, about 900 mg to about 1150 mg, about 900 mg to about 1125 mg, about 900 mg to about 1100 mg, about 900 mg to about 1075 mg, about 900 mg to about 1050 mg, about 900 mg to about 1025 mg, about 900 mg to about 1000 mg, about 900 mg to about 975 mg, about 900 mg to about 950 mg, about 900 mg to about 925 mg, about 925 mg to about 1350 mg, about 925 mg to about 1325 mg, about 925 mg to about 1300 mg, about 925 mg to about 1275 mg, about 925 mg to about 1250 mg, about 925 mg to about 1225 mg, about 925 mg to about 1200 mg, about 925 mg to about 1175 mg, about 925 mg to about 1150 mg, about 925 mg to about 1125 mg, about 925 mg to about 1100 mg, about 925 mg to about 1075 mg, about 925 mg to about 1050 mg, about 925 mg to about 1025 mg, about 925 mg to about 1000 mg, about 925 mg to about 975 mg, about 925 mg to about 950 mg, about 950 mg to about 1350 mg, about 950 mg to about 1325 mg, about 950 mg to about 1300 mg, about 950 mg to about 1275 mg, about 950 mg to about 1250 mg, about 950 mg to about 1225 mg, about 950 mg to about 1200 mg, about 950 mg to about 1175 mg, about 950 mg to about 1150 mg, about 950 mg to about 1125 mg, about 950 mg to about 1100 mg, about 950 mg to about 1075 mg, about 950 mg to about 1050 mg, about 950 mg to about 1025 mg, about 950 mg to about 1000 mg, about 950 mg to about 975 mg, about 975 mg to about 1350 mg, about 975 mg to about 1325 mg, about 975 mg to about 1300 mg, about 975 mg to about 1275 mg, about 975 mg to about 1250 mg, about 975 mg to about 1225 mg, about 975 mg to about 1200 mg, about 975 mg to about 1175 mg, about 975 mg to about 1150 mg, about 975 mg to about 1125 mg, about 975 mg to about 1100 mg, about 975 mg to about 1075 mg, about 975 mg to about 1050 mg, about 975 mg to about 1025 mg, about 975 mg to about 1000 mg, about 1000 mg to about 1350 mg, about 1000 mg to about 1325 mg, about 1000 mg to about 1300 mg, about 1000 mg to about 1275 mg, about 1000 mg to about 1250 mg, about 1000 mg to about 1225 mg, about 1000 mg to about 1200 mg, about 1000 mg to about 1175 mg, about 1000 mg to about 1150 mg, about 1000 mg to about 1125 mg, about 1000 mg to about 1100 mg, about 1000 mg to about 1075 mg, about 1000 mg to about 1050 mg, about 1000 mg to about 1025 mg, about 1025 mg to about 1350 mg, about 1025 mg to about 1325 mg, about 1025 mg to about 1300 mg, about 1025 mg to about 1275 mg, about 1025 mg to about 1250 mg, about 1025 mg to about 1225 mg, about 1025 mg to about 1200 mg, about 1025 mg to about 1175 mg, about 1025 mg to about 1150 mg, about 1025 mg to about 1125 mg, about 1025 mg to about 1100 mg, about 1025 mg to about 1075 mg, about 1025 mg to about 1050 mg, about 1050 mg to about 1350 mg, about 1050 mg to about 1325 mg, about 1050 mg to about 1300 mg, about 1050 mg to about 1275 mg, about 1050 mg to about 1250 mg, about 1050 mg to about 1225 mg, about 1050 mg to about 1200 mg, about 1050 mg to about 1175 mg, about 1050 mg to about 1150 mg, about 1050 mg to about 1125 mg, about 1050 mg to about 1100 mg, about 1050 mg to about 1075 mg, about 1075 mg to about 1350 mg, about 1075 mg to about 1325 mg, about 1075 mg to about 1300 mg, about 1075 mg to about 1275 mg, about 1075 mg to about 1250 mg, about 1075 mg to about 1225 mg, about 1075 mg to about 1200 mg, about 1075 mg to about 1175 mg, about 1075 mg to about 1150 mg, about 1075 mg to about 1125 mg, about 1075 mg to about 1100 mg, about 1100 mg to about 1350 mg, about 1100 mg to about 1325 mg, about 1100 mg to about 1300 mg, about 1100 mg to about 1275 mg, about 1100 mg to about 1250 mg, about 1100 mg to about 1225 mg, about 1100 mg to about 1200 mg, about 1100 mg to about 1175 mg, about 1100 mg to about 1150 mg, about 1100 mg to about 1125 mg, about 1125 mg to about 1350 mg, about 1125 mg to about 1325 mg, about 1125 mg to about 1300 mg, about 1125 mg to about 1275 mg, about 1125 mg to about 1250 mg, about 1125 mg to about 1225 mg, about 1125 mg to about 1200 mg, about 1125 mg to about 1175 mg, about 1125 mg to about 1150 mg, about 1150 mg to about 1350 mg, about 1150 mg to about 1325 mg, about 1150 mg to about 1300 mg, about 1150 mg to about 1275 mg, about 1150 mg to about 1250 mg, about 1150 mg to about 1225 mg, about 1150 mg to about 1200 mg, about 1150 mg to about 1175 mg, about 1175 mg to about 1350 mg, about 1175 mg to about 1325 mg, about 1175 mg to about 1300 mg, about 1175 mg to about 1275 mg, about 1175 mg to about 1250 mg, about 1175 mg to about 1225 mg, about 1175 mg to about 1200 mg, about 1200 mg to about 1350 mg, about 1200 mg to about 1325 mg, about 1200 mg to about 1300 mg, about 1200 mg to about 1275 mg, about 1200 mg to about 1250 mg, about 1200 mg to about 1225 mg, about 1225 mg to about 1350 mg, about 1225 mg to about 1325 mg, about 1225 mg to about 1300 mg, about 1225 mg to about 1275 mg, about 1225 mg to about 1250 mg, about 1250 mg to about 1350 mg, about 1250 mg to about 1325 mg, about 1250 mg to about 1300 mg, about 1250 mg to about 1275 mg, about 1275 mg to about 1350 mg, about 1275 mg to about 1325 mg, about 1275 mg to about 1300 mg, about 1300 mg to about 1350 mg, about 1300 mg to about 1325 mg, or about 1325 mg to about 1350 mg.

In one aspect, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of from about 900 mg to about 1800 mg (e.g., about 1350 mg). The C1D1 may be administered, for example, IV or SC. In some aspects, the C1D1 is administered IV. In other aspects, the C1D1 is administered SC (e.g., by a pump (e.g., by a patch pump)).

In another aspect, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of from about 900 mg to about 1800 mg (e.g., about 1350 mg). In some aspects, the C1D1 is administered IV. In other aspects, the C1D1 is administered SC (e.g., by a pump (e.g., by a patch pump)).

In another aspect, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of from about 900 mg to about 1800 mg (e.g., about 1350 mg). In some aspects, the C1D1 is administered IV. In other aspects, the C1D1 is administered SC (e.g., by a pump (e.g., by a patch pump)).

For example, in any of the preceding aspects, the first dose (C1D1) of the anti-tryptase antibody, and/or any additional doses of the anti-tryptase antibody, may be about 900 mg to about 1800 mg, about 900 mg to about 1775 mg, about 900 mg to about 1750 mg, about 900 mg to about 1725 mg, about 900 mg to about 1700 mg, about 900 mg to about 1675 mg, about 900 mg to about 1650 mg, about 900 mg to about 1625 mg, about 900 mg to about 1600 mg, about 900 mg to about 1575 mg, about 900 mg to about 1550 mg, about 900 mg to about 1525 mg, about 900 mg to about 1500 mg, about 900 mg to about 1475 mg, about 900 mg to about 1450 mg, about 900 mg to about 1425 mg, about 900 mg to about 1400 mg, about 900 mg to about 1375 mg, about 900 mg to about 1350 mg, about 900 mg to about 1325 mg, about 900 mg to about 1300 mg, about 900 mg to about 1275 mg, about 900 mg to about 1250 mg, about 900 mg to about 1225 mg, about 900 mg to about 1200 mg, about 900 mg to about 1175 mg, about 900 mg to about 1150 mg, about 900 mg to about 1125 mg, about 900 mg to about 1100 mg, about 900 mg to about 1075 mg, about 900 mg to about 1050 mg, about 900 mg to about 1025 mg, about 900 mg to about 1000 mg, about 900 mg to about 975 mg, about 900 mg to about 950 mg, about 900 mg to about 925 mg, about 925 mg to about 1800 mg, about 925 mg to about 1775 mg, about 925 mg to about 1750 mg, about 925 mg to about 1725 mg, about 925 mg to about 1700 mg, about 925 mg to about 1675 mg, about 925 mg to about 1650 mg, about 925 mg to about 1625 mg, about 925 mg to about 1600 mg, about 925 mg to about 1575 mg, about 925 mg to about 1550 mg, about 925 mg to about 1525 mg, about 925 mg to about 1500 mg, about 925 mg to about 1475 mg, about 925 mg to about 1450 mg, about 925 mg to about 1425 mg, about 925 mg to about 1400 mg, about 925 mg to about 1375 mg, about 925 mg to about 1350 mg, about 925 mg to about 1325 mg, about 925 mg to about 1300 mg, about 925 mg to about 1275 mg, about 925 mg to about 1250 mg, about 925 mg to about 1225 mg, about 925 mg to about 1200 mg, about 925 mg to about 1175 mg, about 925 mg to about 1150 mg, about 925 mg to about 1125 mg, about 925 mg to about 1100 mg, about 925 mg to about 1075 mg, about 925 mg to about 1050 mg, about 925 mg to about 1025 mg, about 925 mg to about 1000 mg, about 925 mg to about 975 mg, about 925 mg to about 950 mg, about 950 mg to about 1800 mg, about 950 mg to about 1775 mg, about 950 mg to about 1750 mg, about 950 mg to about 1725 mg, about 950 mg to about 1700 mg, about 950 mg to about 1675 mg, about 950 mg to about 1650 mg, about 950 mg to about 1625 mg, about 950 mg to about 1600 mg, about 950 mg to about 1575 mg, about 950 mg to about 1550 mg, about 950 mg to about 1525 mg, about 950 mg to about 1500 mg, about 950 mg to about 1475 mg, about 950 mg to about 1450 mg, about 950 mg to about 1425 mg, about 950 mg to about 1400 mg, about 950 mg to about 1375 mg, about 950 mg to about 1350 mg, about 950 mg to about 1325 mg, about 950 mg to about 1300 mg, about 950 mg to about 1275 mg, about 950 mg to about 1250 mg, about 950 mg to about 1225 mg, about 950 mg to about 1200 mg, about 950 mg to about 1175 mg, about 950 mg to about 1150 mg, about 950 mg to about 1125 mg, about 950 mg to about 1100 mg, about 950 mg to about 1075 mg, about 950 mg to about 1050 mg, about 950 mg to about 1025 mg, about 950 mg to about 1000 mg, about 950 mg to about 975 mg, about 975 mg to about 1800 mg, about 975 mg to about 1775 mg, about 975 mg to about 1750 mg, about 975 mg to about 1725 mg, about 975 mg to about 1700 mg, about 975 mg to about 1675 mg, about 975 mg to about 1650 mg, about 975 mg to about 1625 mg, about 975 mg to about 1600 mg, about 975 mg to about 1575 mg, about 975 mg to about 1550 mg, about 975 mg to about 1525 mg, about 975 mg to about 1500 mg, about 975 mg to about 1475 mg, about 975 mg to about 1450 mg, about 975 mg to about 1425 mg, about 975 mg to about 1400 mg, about 975 mg to about 1375 mg, about 975 mg to about 1350 mg, about 975 mg to about 1325 mg, about 975 mg to about 1300 mg, about 975 mg to about 1275 mg, about 975 mg to about 1250 mg, about 975 mg to about 1225 mg, about 975 mg to about 1200 mg, about 975 mg to about 1175 mg, about 975 mg to about 1150 mg, about 975 mg to about 1125 mg, about 975 mg to about 1100 mg, about 975 mg to about 1075 mg, about 975 mg to about 1050 mg, about 975 mg to about 1025 mg, about 975 mg to about 1000 mg, about 1000 mg to about 1800 mg, about 1000 mg to about 1775 mg, about 1000 mg to about 1750 mg, about 1000 mg to about 1725 mg, about 1000 mg to about 1700 mg, about 1000 mg to about 1675 mg, about 1000 mg to about 1650 mg, about 1000 mg to about 1625 mg, about 1000 mg to about 1600 mg, about 1000 mg to about 1575 mg, about 1000 mg to about 1550 mg, about 1000 mg to about 1525 mg, about 1000 mg to about 1500 mg, about 1000 mg to about 1475 mg, about 1000 mg to about 1450 mg, about 1000 mg to about 1425 mg, about 1000 mg to about 1400 mg, about 1000 mg to about 1375 mg, about 1000 mg to about 1350 mg, about 1000 mg to about 1325 mg, about 1000 mg to about 1300 mg, about 1000 mg to about 1275 mg, about 1000 mg to about 1250 mg, about 1000 mg to about 1225 mg, about 1000 mg to about 1200 mg, about 1000 mg to about 1175 mg, about 1000 mg to about 1150 mg, about 1000 mg to about 1125 mg, about 1000 mg to about 1100 mg, about 1000 mg to about 1075 mg, about 1000 mg to about 1050 mg, about 1000 mg to about 1025 mg, about 1025 mg to about 1800 mg, about 1025 mg to about 1775 mg, about 1025 mg to about 1750 mg, about 1025 mg to about 1725 mg, about 1025 mg to about 1700 mg, about 1025 mg to about 1675 mg, about 1025 mg to about 1650 mg, about 1025 mg to about 1625 mg, about 1025 mg to about 1600 mg, about 1025 mg to about 1575 mg, about 1025 mg to about 1550 mg, about 1025 mg to about 1525 mg, about 1025 mg to about 1500 mg, about 1025 mg to about 1475 mg, about 1025 mg to about 1450 mg, about 1025 mg to about 1425 mg, about 1025 mg to about 1400 mg, about 1025 mg to about 1375 mg, about 1025 mg to about 1350 mg, about 1025 mg to about 1325 mg, about 1025 mg to about 1300 mg, about 1025 mg to about 1275 mg, about 1025 mg to about 1250 mg, about 1025 mg to about 1225 mg, about 1025 mg to about 1200 mg, about 1025 mg to about 1175 mg, about 1025 mg to about 1150 mg, about 1025 mg to about 1125 mg, about 1025 mg to about 1100 mg, about 1025 mg to about 1075 mg, about 1025 mg to about 1050 mg, about 1050 mg to about 1800 mg, about 1050 mg to about 1775 mg, about 1050 mg to about 1750 mg, about 1050 mg to about 1725 mg, about 1050 mg to about 1700 mg, about 1050 mg to about 1675 mg, about 1050 mg to about 1650 mg, about 1050 mg to about 1625 mg, about 1050 mg to about 1600 mg, about 1050 mg to about 1575 mg, about 1050 mg to about 1550 mg, about 1050 mg to about 1525 mg, about 1050 mg to about 1500 mg, about 1050 mg to about 1475 mg, about 1050 mg to about 1450 mg, about 1050 mg to about 1425 mg, about 1050 mg to about 1400 mg, about 1050 mg to about 1375 mg, about 1050 mg to about 1350 mg, about 1050 mg to about 1325 mg, about 1050 mg to about 1300 mg, about 1050 mg to about 1275 mg, about 1050 mg to about 1250 mg, about 1050 mg to about 1225 mg, about 1050 mg to about 1200 mg, about 1050 mg to about 1175 mg, about 1050 mg to about 1150 mg, about 1050 mg to about 1125 mg, about 1050 mg to about 1100 mg, about 1050 mg to about 1075 mg, about 1075 mg to about 1800 mg, about 1075 mg to about 1775 mg, about 1075 mg to about 1750 mg, about 1075 mg to about 1725 mg, about 1075 mg to about 1700 mg, about 1075 mg to about 1675 mg, about 1075 mg to about 1650 mg, about 1075 mg to about 1625 mg, about 1075 mg to about 1600 mg, about 1075 mg to about 1575 mg, about 1075 mg to about 1550 mg, about 1075 mg to about 1525 mg, about 1075 mg to about 1500 mg, about 1075 mg to about 1475 mg, about 1075 mg to about 1450 mg, about 1075 mg to about 1425 mg, about 1075 mg to about 1400 mg, about 1075 mg to about 1375 mg, about 1075 mg to about 1350 mg, about 1075 mg to about 1325 mg, about 1075 mg to about 1300 mg, about 1075 mg to about 1275 mg, about 1075 mg to about 1250 mg, about 1075 mg to about 1225 mg, about 1075 mg to about 1200 mg, about 1075 mg to about 1175 mg, about 1075 mg to about 1150 mg, about 1075 mg to about 1125 mg, about 1075 mg to about 1100 mg, about 1100 mg to about 1800 mg, about 1100 mg to about 1775 mg, about 1100 mg to about 1750 mg, about 1100 mg to about 1725 mg, about 1100 mg to about 1700 mg, about 1100 mg to about 1675 mg, about 1100 mg to about 1650 mg, about 1100 mg to about 1625 mg, about 1100 mg to about 1600 mg, about 1100 mg to about 1575 mg, about 1100 mg to about 1550 mg, about 1100 mg to about 1525 mg, about 1100 mg to about 1500 mg, about 1100 mg to about 1475 mg, about 1100 mg to about 1450 mg, about 1100 mg to about 1425 mg, about 1100 mg to about 1400 mg, about 1100 mg to about 1375 mg, about 1100 mg to about 1350 mg, about 1100 mg to about 1325 mg, about 1100 mg to about 1300 mg, about 1100 mg to about 1275 mg, about 1100 mg to about 1250 mg, about 1100 mg to about 1225 mg, about 1100 mg to about 1200 mg, about 1100 mg to about 1175 mg, about 1100 mg to about 1150 mg, about 1100 mg to about 1125 mg, about 1125 mg to about 1800 mg, about 1125 mg to about 1775 mg, about 1125 mg to about 1750 mg, about 1125 mg to about 1725 mg, about 1125 mg to about 1700 mg, about 1125 mg to about 1675 mg, about 1125 mg to about 1650 mg, about 1125 mg to about 1625 mg, about 1125 mg to about 1600 mg, about 1125 mg to about 1575 mg, about 1125 mg to about 1550 mg, about 1125 mg to about 1525 mg, about 1125 mg to about 1500 mg, about 1125 mg to about 1475 mg, about 1125 mg to about 1450 mg, about 1125 mg to about 1425 mg, about 1125 mg to about 1400 mg, about 1125 mg to about 1375 mg, about 1125 mg to about 1350 mg, about 1125 mg to about 1325 mg, about 1125 mg to about 1300 mg, about 1125 mg to about 1275 mg, about 1125 mg to about 1250 mg, about 1125 mg to about 1225 mg, about 1125 mg to about 1200 mg, about 1125 mg to about 1175 mg, about 1125 mg to about 1150 mg, about 1150 mg to about 1800 mg, about 1150 mg to about 1775 mg, about 1150 mg to about 1750 mg, about 1150 mg to about 1725 mg, about 1150 mg to about 1700 mg, about 1150 mg to about 1675 mg, about 1150 mg to about 1650 mg, about 1150 mg to about 1625 mg, about 1150 mg to about 1600 mg, about 1150 mg to about 1575 mg, about 1150 mg to about 1550 mg, about 1150 mg to about 1525 mg, about 1150 mg to about 1500 mg, about 1150 mg to about 1475 mg, about 1150 mg to about 1450 mg, about 1150 mg to about 1425 mg, about 1150 mg to about 1400 mg, about 1150 mg to about 1375 mg, about 1150 mg to about 1350 mg, about 1150 mg to about 1325 mg, about 1150 mg to about 1300 mg, about 1150 mg to about 1275 mg, about 1150 mg to about 1250 mg, about 1150 mg to about 1225 mg, about 1150 mg to about 1200 mg, about 1150 mg to about 1175 mg, about 1175 mg to about 1800 mg, about 1175 mg to about 1775 mg, about 1175 mg to about 1750 mg, about 1175 mg to about 1725 mg, about 1175 mg to about 1700 mg, about 1175 mg to about 1675 mg, about 1175 mg to about 1650 mg, about 1175 mg to about 1625 mg, about 1175 mg to about 1600 mg, about 1175 mg to about 1575 mg, about 1175 mg to about 1550 mg, about 1175 mg to about 1525 mg, about 1175 mg to about 1500 mg, about 1175 mg to about 1475 mg, about 1175 mg to about 1450 mg, about 1175 mg to about 1425 mg, about 1175 mg to about 1400 mg, about 1175 mg to about 1375 mg, about 1175 mg to about 1350 mg, about 1175 mg to about 1325 mg, about 1175 mg to about 1300 mg, about 1175 mg to about 1275 mg, about 1175 mg to about 1250 mg, about 1175 mg to about 1225 mg, about 1175 mg to about 1200 mg, about 1200 mg to about 1800 mg, about 1200 mg to about 1775 mg, about 1200 mg to about 1750 mg, about 1200 mg to about 1725 mg, about 1200 mg to about 1700 mg, about 1200 mg to about 1675 mg, about 1200 mg to about 1650 mg, about 1200 mg to about 1625 mg, about 1200 mg to about 1600 mg, about 1200 mg to about 1575 mg, about 1200 mg to about 1550 mg, about 1200 mg to about 1525 mg, about 1200 mg to about 1500 mg, about 1200 mg to about 1475 mg, about 1200 mg to about 1450 mg, about 1200 mg to about 1425 mg, about 1200 mg to about 1400 mg, about 1200 mg to about 1375 mg, about 1200 mg to about 1350 mg, about 1200 mg to about 1325 mg, about 1200 mg to about 1300 mg, about 1200 mg to about 1275 mg, about 1200 mg to about 1250 mg, about 1200 mg to about 1225 mg, about 1225 mg to about 1800 mg, about 1225 mg to about 1775 mg, about 1225 mg to about 1750 mg, about 1225 mg to about 1725 mg, about 1225 mg to about 1700 mg, about 1225 mg to about 1675 mg, about 1225 mg to about 1650 mg, about 1225 mg to about 1625 mg, about 1225 mg to about 1600 mg, about 1225 mg to about 1575 mg, about 1225 mg to about 1550 mg, about 1225 mg to about 1525 mg, about 1225 mg to about 1500 mg, about 1225 mg to about 1475 mg, about 1225 mg to about 1450 mg, about 1225 mg to about 1425 mg, about 1225 mg to about 1400 mg, about 1225 mg to about 1375 mg, about 1225 mg to about 1350 mg, about 1225 mg to about 1325 mg, about 1225 mg to about 1300 mg, about 1225 mg to about 1275 mg, about 1225 mg to about 1250 mg, about 1250 mg to about 1800 mg, about 1250 mg to about 1775 mg, about 1250 mg to about 1750 mg, about 1250 mg to about 1725 mg, about 1250 mg to about 1700 mg, about 1250 mg to about 1675 mg, about 1250 mg to about 1650 mg, about 1250 mg to about 1625 mg, about 1250 mg to about 1600 mg, about 1250 mg to about 1575 mg, about 1250 mg to about 1550 mg, about 1250 mg to about 1525 mg, about 1250 mg to about 1500 mg, about 1250 mg to about 1475 mg, about 1250 mg to about 1450 mg, about 1250 mg to about 1425 mg, about 1250 mg to about 1400 mg, about 1250 mg to about 1375 mg, about 1250 mg to about 1350 mg, about 1250 mg to about 1325 mg, about 1250 mg to about 1300 mg, about 1250 mg to about 1275 mg, about 1275 mg to about 1800 mg, about 1275 mg to about 1775 mg, about 1275 mg to about 1750 mg, about 1275 mg to about 1725 mg, about 1275 mg to about 1700 mg, about 1275 mg to about 1675 mg, about 1275 mg to about 1650 mg, about 1275 mg to about 1625 mg, about 1275 mg to about 1600 mg, about 1275 mg to about 1575 mg, about 1275 mg to about 1550 mg, about 1275 mg to about 1525 mg, about 1275 mg to about 1500 mg, about 1275 mg to about 1475 mg, about 1275 mg to about 1450 mg, about 1275 mg to about 1425 mg, about 1275 mg to about 1400 mg, about 1275 mg to about 1375 mg, about 1275 mg to about 1350 mg, about 1275 mg to about 1325 mg, about 1275 mg to about 1300 mg, about 1300 mg to about 1800 mg, about 1300 mg to about 1775 mg, about 1300 mg to about 1750 mg, about 1300 mg to about 1725 mg, about 1300 mg to about 1700 mg, about 1300 mg to about 1675 mg, about 1300 mg to about 1650 mg, about 1300 mg to about 1625 mg, about 1300 mg to about 1600 mg, about 1300 mg to about 1575 mg, about 1300 mg to about 1550 mg, about 1300 mg to about 1525 mg, about 1300 mg to about 1500 mg, about 1300 mg to about 1475 mg, about 1300 mg to about 1450 mg, about 1300 mg to about 1425 mg, about 1300 mg to about 1400 mg, about 1300 mg to about 1375 mg, about 1300 mg to about 1350 mg, about 1300 mg to about 1325 mg, about 1325 mg to about 1800 mg, about 1325 mg to about 1775 mg, about 1325 mg to about 1750 mg, about 1325 mg to about 1725 mg, about 1325 mg to about 1700 mg, about 1325 mg to about 1675 mg, about 1325 mg to about 1650 mg, about 1325 mg to about 1625 mg, about 1325 mg to about 1600 mg, about 1325 mg to about 1575 mg, about 1325 mg to about 1550 mg, about 1325 mg to about 1525 mg, about 1325 mg to about 1500 mg, about 1325 mg to about 1475 mg, about 1325 mg to about 1450 mg, about 1325 mg to about 1425 mg, about 1325 mg to about 1400 mg, about 1325 mg to about 1375 mg, about 1325 mg to about 1350 mg, about 1350 mg to about 1800 mg, about 1350 mg to about 1775 mg, about 1350 mg to about 1750 mg, about 1350 mg to about 1725 mg, about 1350 mg to about 1700 mg, about 1350 mg to about 1675 mg, about 1350 mg to about 1650 mg, about 1350 mg to about 1625 mg, about 1350 mg to about 1600 mg, about 1350 mg to about 1575 mg, about 1350 mg to about 1550 mg, about 1350 mg to about 1525 mg, about 1350 mg to about 1500 mg, about 1350 mg to about 1475 mg, about 1350 mg to about 1450 mg, about 1350 mg to about 1425 mg, about 1350 mg to about 1400 mg, about 1350 mg to about 1375 mg, about 1375 mg to about 1800 mg, about 1375 mg to about 1775 mg, about 1375 mg to about 1750 mg, about 1375 mg to about 1725 mg, about 1375 mg to about 1700 mg, about 1375 mg to about 1675 mg, about 1375 mg to about 1650 mg, about 1375 mg to about 1625 mg, about 1375 mg to about 1600 mg, about 1375 mg to about 1575 mg, about 1375 mg to about 1550 mg, about 1375 mg to about 1525 mg, about 1375 mg to about 1500 mg, about 1375 mg to about 1475 mg, about 1375 mg to about 1450 mg, about 1375 mg to about 1425 mg, about 1375 mg to about 1400 mg, about 1400 mg to about 1800 mg, about 1400 mg to about 1775 mg, about 1400 mg to about 1750 mg, about 1400 mg to about 1725 mg, about 1400 mg to about 1700 mg, about 1400 mg to about 1675 mg, about 1400 mg to about 1650 mg, about 1400 mg to about 1625 mg, about 1400 mg to about 1600 mg, about 1400 mg to about 1575 mg, about 1400 mg to about 1550 mg, about 1400 mg to about 1525 mg, about 1400 mg to about 1500 mg, about 1400 mg to about 1475 mg, about 1400 mg to about 1450 mg, about 1400 mg to about 1425 mg, about 1425 mg to about 1800 mg, about 1425 mg to about 1775 mg, about 1425 mg to about 1750 mg, about 1425 mg to about 1725 mg, about 1425 mg to about 1700 mg, about 1425 mg to about 1675 mg, about 1425 mg to about 1650 mg, about 1425 mg to about 1625 mg, about 1425 mg to about 1600 mg, about 1425 mg to about 1575 mg, about 1425 mg to about 1550 mg, about 1425 mg to about 1525 mg, about 1425 mg to about 1500 mg, about 1425 mg to about 1475 mg, about 1425 mg to about 1450 mg, about 1450 mg to about 1800 mg, about 1450 mg to about 1775 mg, about 1450 mg to about 1750 mg, about 1450 mg to about 1725 mg, about 1450 mg to about 1700 mg, about 1450 mg to about 1675 mg, about 1450 mg to about 1650 mg, about 1450 mg to about 1625 mg, about 1450 mg to about 1600 mg, about 1450 mg to about 1575 mg, about 1450 mg to about 1550 mg, about 1450 mg to about 1525 mg, about 1450 mg to about 1500 mg, about 1450 mg to about 1475 mg, about 1475 mg to about 1800 mg, about 1475 mg to about 1775 mg, about 1475 mg to about 1750 mg, about 1475 mg to about 1725 mg, about 1475 mg to about 1700 mg, about 1475 mg to about 1675 mg, about 1475 mg to about 1650 mg, about 1475 mg to about 1625 mg, about 1475 mg to about 1600 mg, about 1475 mg to about 1575 mg, about 1475 mg to about 1550 mg, about 1475 mg to about 1525 mg, about 1475 mg to about 1500 mg, about 1500 mg to about 1800 mg, about 1500 mg to about 1775 mg, about 1500 mg to about 1750 mg, about 1500 mg to about 1725 mg, about 1500 mg to about 1700 mg, about 1500 mg to about 1675 mg, about 1500 mg to about 1650 mg, about 1500 mg to about 1625 mg, about 1500 mg to about 1600 mg, about 1500 mg to about 1575 mg, about 1500 mg to about 1550 mg, about 1500 mg to about 1525 mg, about 1525 mg to about 1800 mg, about 1525 mg to about 1775 mg, about 1525 mg to about 1750 mg, about 1525 mg to about 1725 mg, about 1525 mg to about 1700 mg, about 1525 mg to about 1675 mg, about 1525 mg to about 1650 mg, about 1525 mg to about 1625 mg, about 1525 mg to about 1600 mg, about 1525 mg to about 1575 mg, about 1525 mg to about 1550 mg, about 1550 mg to about 1800 mg, about 1550 mg to about 1775 mg, about 1550 mg to about 1750 mg, about 1550 mg to about 1725 mg, about 1550 mg to about 1700 mg, about 1550 mg to about 1675 mg, about 1550 mg to about 1650 mg, about 1550 mg to about 1625 mg, about 1550 mg to about 1600 mg, about 1550 mg to about 1575 mg, about 1575 mg to about 1800 mg, about 1575 mg to about 1775 mg, about 1575 mg to about 1750 mg, about 1575 mg to about 1725 mg, about 1575 mg to about 1700 mg, about 1575 mg to about 1675 mg, about 1575 mg to about 1650 mg, about 1575 mg to about 1625 mg, about 1575 mg to about 1600 mg, about 1600 mg to about 1800 mg, about 1600 mg to about 1775 mg, about 1600 mg to about 1750 mg, about 1600 mg to about 1725 mg, about 1600 mg to about 1700 mg, about 1600 mg to about 1675 mg, about 1600 mg to about 1650 mg, about 1600 mg to about 1625 mg, about 1625 mg to about 1800 mg, about 1625 mg to about 1775 mg, about 1625 mg to about 1750 mg, about 1625 mg to about 1725 mg, about 1625 mg to about 1700 mg, about 1625 mg to about 1675 mg, about 1625 mg to about 1650 mg, about 1650 mg to about 1800 mg, about 1650 mg to about 1775 mg, about 1650 mg to about 1750 mg, about 1650 mg to about 1725 mg, about 1650 mg to about 1700 mg, about 1650 mg to about 1675 mg, about 1675 mg to about 1800 mg, about 1675 mg to about 1775 mg, about 1675 mg to about 1750 mg, about 1675 mg to about 1725 mg, about 1675 mg to about 1700 mg, about 1700 mg to about 1800 mg, about 1700 mg to about 1775 mg, about 1700 mg to about 1750 mg, about 1700 mg to about 1725 mg, about 1725 mg to about 1800 mg, about 1725 mg to about 1775 mg, about 1725 mg to about 1750 mg, about 1750 mg to about 1800 mg, about 1750 mg to about 1775 mg, or about 1775 mg to about 1800 mg.

In one aspect, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of from about 1350 mg to about 3600 mg (e.g., about 1800 mg). The C1D1 may be administered, for example, IV or SC. In some aspects, the C1D1 is administered IV. In other aspects, the C1D1 is administered SC (e.g., by a pump (e.g., by a patch pump)).

In another aspect, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of from about 1350 mg to about 3600 mg (e.g., about 1800 mg). In some aspects, the C1D1 is administered IV. In other aspects, the C1D1 is administered SC (e.g., by a pump (e.g., by a patch pump)).

In another aspect, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of from about 1350 mg to about 3600 mg (e.g., about 1800 mg). In some aspects, the C1D1 is administered IV. In other aspects, the C1D1 is administered SC (e.g., by a pump (e.g., by a patch pump)).

For example, in any of the preceding aspects, the first dose (C1D1) of the anti-tryptase antibody, and/or any additional doses of the anti-tryptase antibody, may be about 1350 mg to about 3600 mg, about 1350 mg to about 3550 mg, about 1350 mg to about 3500 mg, about 1350 mg to about 3450 mg, about 1350 mg to about 3400 mg, about 1350 mg to about 3350 mg, about 1350 mg to about 3300 mg, about 1350 mg to about 3250 mg, about 1350 mg to about 3200 mg, about 1350 mg to about 3150 mg, about 1350 mg to about 3100 mg, about 1350 mg to about 3050 mg, about 1350 mg to about 3000 mg, about 1350 mg to about 2950 mg, about 1350 mg to about 2900 mg, about 1350 mg to about 2850 mg, about 1350 mg to about 2800 mg, about 1350 mg to about 2750 mg, about 1350 mg to about 2700 mg, about 1350 mg to about 2650 mg, about 1350 mg to about 2600 mg, about 1350 mg to about 2550 mg, about 1350 mg to about 2500 mg, about 1350 mg to about 2450 mg, about 1350 mg to about 2400 mg, about 1350 mg to about 2350 mg, about 1350 mg to about 2300 mg, about 1350 mg to about 2250 mg, about 1350 mg to about 2200 mg, about 1350 mg to about 2150 mg, about 1350 mg to about 2100 mg, about 1350 mg to about 2050 mg, about 1350 mg to about 2000 mg, about 1350 mg to about 1950 mg, about 1350 mg to about 1900 mg, about 1350 mg to about 1850 mg, about 1350 mg to about 1800 mg, about 1350 mg to about 1750 mg, about 1350 mg to about 1700 mg, about 1350 mg to about 1650 mg, about 1350 mg to about 1600 mg, about 1350 mg to about 1550 mg, about 1350 mg to about 1500 mg, about 1350 mg to about 1450 mg, about 1350 mg to about 1400 mg, about 1400 mg to about 3600 mg, about 1400 mg to about 3550 mg, about 1400 mg to about 3500 mg, about 1400 mg to about 3450 mg, about 1400 mg to about 3400 mg, about 1400 mg to about 3350 mg, about 1400 mg to about 3300 mg, about 1400 mg to about 3250 mg, about 1400 mg to about 3200 mg, about 1400 mg to about 3150 mg, about 1400 mg to about 3100 mg, about 1400 mg to about 3050 mg, about 1400 mg to about 3000 mg, about 1400 mg to about 2950 mg, about 1400 mg to about 2900 mg, about 1400 mg to about 2850 mg, about 1400 mg to about 2800 mg, about 1400 mg to about 2750 mg, about 1400 mg to about 2700 mg, about 1400 mg to about 2650 mg, about 1400 mg to about 2600 mg, about 1400 mg to about 2550 mg, about 1400 mg to about 2500 mg, about 1400 mg to about 2450 mg, about 1400 mg to about 2400 mg, about 1400 mg to about 2350 mg, about 1400 mg to about 2300 mg, about 1400 mg to about 2250 mg, about 1400 mg to about 2200 mg, about 1400 mg to about 2150 mg, about 1400 mg to about 2100 mg, about 1400 mg to about 2050 mg, about 1400 mg to about 2000 mg, about 1400 mg to about 1950 mg, about 1400 mg to about 1900 mg, about 1400 mg to about 1850 mg, about 1400 mg to about 1800 mg, about 1400 mg to about 1750 mg, about 1400 mg to about 1700 mg, about 1400 mg to about 1650 mg, about 1400 mg to about 1600 mg, about 1400 mg to about 1550 mg, about 1400 mg to about 1500 mg, about 1400 mg to about 1450 mg, about 1450 mg to about 3600 mg, about 1450 mg to about 3550 mg, about 1450 mg to about 3500 mg, about 1450 mg to about 3450 mg, about 1450 mg to about 3400 mg, about 1450 mg to about 3350 mg, about 1450 mg to about 3300 mg, about 1450 mg to about 3250 mg, about 1450 mg to about 3200 mg, about 1450 mg to about 3150 mg, about 1450 mg to about 3100 mg, about 1450 mg to about 3050 mg, about 1450 mg to about 3000 mg, about 1450 mg to about 2950 mg, about 1450 mg to about 2900 mg, about 1450 mg to about 2850 mg, about 1450 mg to about 2800 mg, about 1450 mg to about 2750 mg, about 1450 mg to about 2700 mg, about 1450 mg to about 2650 mg, about 1450 mg to about 2600 mg, about 1450 mg to about 2550 mg, about 1450 mg to about 2500 mg, about 1450 mg to about 2450 mg, about 1450 mg to about 2400 mg, about 1450 mg to about 2350 mg, about 1450 mg to about 2300 mg, about 1450 mg to about 2250 mg, about 1450 mg to about 2200 mg, about 1450 mg to about 2150 mg, about 1450 mg to about 2100 mg, about 1450 mg to about 2050 mg, about 1450 mg to about 2000 mg, about 1450 mg to about 1950 mg, about 1450 mg to about 1900 mg, about 1450 mg to about 1850 mg, about 1450 mg to about 1800 mg, about 1450 mg to about 1750 mg, about 1450 mg to about 1700 mg, about 1450 mg to about 1650 mg, about 1450 mg to about 1600 mg, about 1450 mg to about 1550 mg, about 1450 mg to about 1500 mg, about 1500 mg to about 3600 mg, about 1500 mg to about 3550 mg, about 1500 mg to about 3500 mg, about 1500 mg to about 3450 mg, about 1500 mg to about 3400 mg, about 1500 mg to about 3350 mg, about 1500 mg to about 3300 mg, about 1500 mg to about 3250 mg, about 1500 mg to about 3200 mg, about 1500 mg to about 3150 mg, about 1500 mg to about 3100 mg, about 1500 mg to about 3050 mg, about 1500 mg to about 3000 mg, about 1500 mg to about 2950 mg, about 1500 mg to about 2900 mg, about 1500 mg to about 2850 mg, about 1500 mg to about 2800 mg, about 1500 mg to about 2750 mg, about 1500 mg to about 2700 mg, about 1500 mg to about 2650 mg, about 1500 mg to about 2600 mg, about 1500 mg to about 2550 mg, about 1500 mg to about 2500 mg, about 1500 mg to about 2450 mg, about 1500 mg to about 2400 mg, about 1500 mg to about 2350 mg, about 1500 mg to about 2300 mg, about 1500 mg to about 2250 mg, about 1500 mg to about 2200 mg, about 1500 mg to about 2150 mg, about 1500 mg to about 2100 mg, about 1500 mg to about 2050 mg, about 1500 mg to about 2000 mg, about 1500 mg to about 1950 mg, about 1500 mg to about 1900 mg, about 1500 mg to about 1850 mg, about 1500 mg to about 1800 mg, about 1500 mg to about 1750 mg, about 1500 mg to about 1700 mg, about 1500 mg to about 1650 mg, about 1500 mg to about 1600 mg, about 1500 mg to about 1550 mg, about 1550 mg to about 3600 mg, about 1550 mg to about 3550 mg, about 1550 mg to about 3500 mg, about 1550 mg to about 3450 mg, about 1550 mg to about 3400 mg, about 1550 mg to about 3350 mg, about 1550 mg to about 3300 mg, about 1550 mg to about 3250 mg, about 1550 mg to about 3200 mg, about 1550 mg to about 3150 mg, about 1550 mg to about 3100 mg, about 1550 mg to about 3050 mg, about 1550 mg to about 3000 mg, about 1550 mg to about 2950 mg, about 1550 mg to about 2900 mg, about 1550 mg to about 2850 mg, about 1550 mg to about 2800 mg, about 1550 mg to about 2750 mg, about 1550 mg to about 2700 mg, about 1550 mg to about 2650 mg, about 1550 mg to about 2600 mg, about 1550 mg to about 2550 mg, about 1550 mg to about 2500 mg, about 1550 mg to about 2450 mg, about 1550 mg to about 2400 mg, about 1550 mg to about 2350 mg, about 1550 mg to about 2300 mg, about 1550 mg to about 2250 mg, about 1550 mg to about 2200 mg, about 1550 mg to about 2150 mg, about 1550 mg to about 2100 mg, about 1550 mg to about 2050 mg, about 1550 mg to about 2000 mg, about 1550 mg to about 1950 mg, about 1550 mg to about 1900 mg, about 1550 mg to about 1850 mg, about 1550 mg to about 1800 mg, about 1550 mg to about 1750 mg, about 1550 mg to about 1700 mg, about 1550 mg to about 1650 mg, about 1550 mg to about 1600 mg, about 1600 mg to about 3600 mg, about 1600 mg to about 3550 mg, about 1600 mg to about 3500 mg, about 1600 mg to about 3450 mg, about 1600 mg to about 3400 mg, about 1600 mg to about 3350 mg, about 1600 mg to about 3300 mg, about 1600 mg to about 3250 mg, about 1600 mg to about 3200 mg, about 1600 mg to about 3150 mg, about 1600 mg to about 3100 mg, about 1600 mg to about 3050 mg, about 1600 mg to about 3000 mg, about 1600 mg to about 2950 mg, about 1600 mg to about 2900 mg, about 1600 mg to about 2850 mg, about 1600 mg to about 2800 mg, about 1600 mg to about 2750 mg, about 1600 mg to about 2700 mg, about 1600 mg to about 2650 mg, about 1600 mg to about 2600 mg, about 1600 mg to about 2550 mg, about 1600 mg to about 2500 mg, about 1600 mg to about 2450 mg, about 1600 mg to about 2400 mg, about 1600 mg to about 2350 mg, about 1600 mg to about 2300 mg, about 1600 mg to about 2250 mg, about 1600 mg to about 2200 mg, about 1600 mg to about 2150 mg, about 1600 mg to about 2100 mg, about 1600 mg to about 2050 mg, about 1600 mg to about 2000 mg, about 1600 mg to about 1950 mg, about 1600 mg to about 1900 mg, about 1600 mg to about 1850 mg, about 1600 mg to about 1800 mg, about 1600 mg to about 1750 mg, about 1600 mg to about 1700 mg, about 1600 mg to about 1650 mg, about 1650 mg to about 3600 mg, about 1650 mg to about 3550 mg, about 1650 mg to about 3500 mg, about 1650 mg to about 3450 mg, about 1650 mg to about 3400 mg, about 1650 mg to about 3350 mg, about 1650 mg to about 3300 mg, about 1650 mg to about 3250 mg, about 1650 mg to about 3200 mg, about 1650 mg to about 3150 mg, about 1650 mg to about 3100 mg, about 1650 mg to about 3050 mg, about 1650 mg to about 3000 mg, about 1650 mg to about 2950 mg, about 1650 mg to about 2900 mg, about 1650 mg to about 2850 mg, about 1650 mg to about 2800 mg, about 1650 mg to about 2750 mg, about 1650 mg to about 2700 mg, about 1650 mg to about 2650 mg, about 1650 mg to about 2600 mg, about 1650 mg to about 2550 mg, about 1650 mg to about 2500 mg, about 1650 mg to about 2450 mg, about 1650 mg to about 2400 mg, about 1650 mg to about 2350 mg, about 1650 mg to about 2300 mg, about 1650 mg to about 2250 mg, about 1650 mg to about 2200 mg, about 1650 mg to about 2150 mg, about 1650 mg to about 2100 mg, about 1650 mg to about 2050 mg, about 1650 mg to about 2000 mg, about 1650 mg to about 1950 mg, about 1650 mg to about 1900 mg, about 1650 mg to about 1850 mg, about 1650 mg to about 1800 mg, about 1650 mg to about 1750 mg, about 1650 mg to about 1700 mg, about 1700 mg to about 3600 mg, about 1700 mg to about 3550 mg, about 1700 mg to about 3500 mg, about 1700 mg to about 3450 mg, about 1700 mg to about 3400 mg, about 1700 mg to about 3350 mg, about 1700 mg to about 3300 mg, about 1700 mg to about 3250 mg, about 1700 mg to about 3200 mg, about 1700 mg to about 3150 mg, about 1700 mg to about 3100 mg, about 1700 mg to about 3050 mg, about 1700 mg to about 3000 mg, about 1700 mg to about 2950 mg, about 1700 mg to about 2900 mg, about 1700 mg to about 2850 mg, about 1700 mg to about 2800 mg, about 1700 mg to about 2750 mg, about 1700 mg to about 2700 mg, about 1700 mg to about 2650 mg, about 1700 mg to about 2600 mg, about 1700 mg to about 2550 mg, about 1700 mg to about 2500 mg, about 1700 mg to about 2450 mg, about 1700 mg to about 2400 mg, about 1700 mg to about 2350 mg, about 1700 mg to about 2300 mg, about 1700 mg to about 2250 mg, about 1700 mg to about 2200 mg, about 1700 mg to about 2150 mg, about 1700 mg to about 2100 mg, about 1700 mg to about 2050 mg, about 1700 mg to about 2000 mg, about 1700 mg to about 1950 mg, about 1700 mg to about 1900 mg, about 1700 mg to about 1850 mg, about 1700 mg to about 1800 mg, about 1700 mg to about 1750 mg, about 1750 mg to about 3600 mg, about 1750 mg to about 3550 mg, about 1750 mg to about 3500 mg, about 1750 mg to about 3450 mg, about 1750 mg to about 3400 mg, about 1750 mg to about 3350 mg, about 1750 mg to about 3300 mg, about 1750 mg to about 3250 mg, about 1750 mg to about 3200 mg, about 1750 mg to about 3150 mg, about 1750 mg to about 3100 mg, about 1750 mg to about 3050 mg, about 1750 mg to about 3000 mg, about 1750 mg to about 2950 mg, about 1750 mg to about 2900 mg, about 1750 mg to about 2850 mg, about 1750 mg to about 2800 mg, about 1750 mg to about 2750 mg, about 1750 mg to about 2700 mg, about 1750 mg to about 2650 mg, about 1750 mg to about 2600 mg, about 1750 mg to about 2550 mg, about 1750 mg to about 2500 mg, about 1750 mg to about 2450 mg, about 1750 mg to about 2400 mg, about 1750 mg to about 2350 mg, about 1750 mg to about 2300 mg, about 1750 mg to about 2250 mg, about 1750 mg to about 2200 mg, about 1750 mg to about 2150 mg, about 1750 mg to about 2100 mg, about 1750 mg to about 2050 mg, about 1750 mg to about 2000 mg, about 1750 mg to about 1950 mg, about 1750 mg to about 1900 mg, about 1750 mg to about 1850 mg, about 1750 mg to about 1800 mg, about 1800 mg to about 3600 mg, about 1800 mg to about 3550 mg, about 1800 mg to about 3500 mg, about 1800 mg to about 3450 mg, about 1800 mg to about 3400 mg, about 1800 mg to about 3350 mg, about 1800 mg to about 3300 mg, about 1800 mg to about 3250 mg, about 1800 mg to about 3200 mg, about 1800 mg to about 3150 mg, about 1800 mg to about 3100 mg, about 1800 mg to about 3050 mg, about 1800 mg to about 3000 mg, about 1800 mg to about 2950 mg, about 1800 mg to about 2900 mg, about 1800 mg to about 2850 mg, about 1800 mg to about 2800 mg, about 1800 mg to about 2750 mg, about 1800 mg to about 2700 mg, about 1800 mg to about 2650 mg, about 1800 mg to about 2600 mg, about 1800 mg to about 2550 mg, about 1800 mg to about 2500 mg, about 1800 mg to about 2450 mg, about 1800 mg to about 2400 mg, about 1800 mg to about 2350 mg, about 1800 mg to about 2300 mg, about 1800 mg to about 2250 mg, about 1800 mg to about 2200 mg, about 1800 mg to about 2150 mg, about 1800 mg to about 2100 mg, about 1800 mg to about 2050 mg, about 1800 mg to about 2000 mg, about 1800 mg to about 1950 mg, about 1800 mg to about 1900 mg, about 1800 mg to about 1850 mg, about 1850 mg to about 3600 mg, about 1850 mg to about 3550 mg, about 1850 mg to about 3500 mg, about 1850 mg to about 3450 mg, about 1850 mg to about 3400 mg, about 1850 mg to about 3350 mg, about 1850 mg to about 3300 mg, about 1850 mg to about 3250 mg, about 1850 mg to about 3200 mg, about 1850 mg to about 3150 mg, about 1850 mg to about 3100 mg, about 1850 mg to about 3050 mg, about 1850 mg to about 3000 mg, about 1850 mg to about 2950 mg, about 1850 mg to about 2900 mg, about 1850 mg to about 2850 mg, about 1850 mg to about 2800 mg, about 1850 mg to about 2750 mg, about 1850 mg to about 2700 mg, about 1850 mg to about 2650 mg, about 1850 mg to about 2600 mg, about 1850 mg to about 2550 mg, about 1850 mg to about 2500 mg, about 1850 mg to about 2450 mg, about 1850 mg to about 2400 mg, about 1850 mg to about 2350 mg, about 1850 mg to about 2300 mg, about 1850 mg to about 2250 mg, about 1850 mg to about 2200 mg, about 1850 mg to about 2150 mg, about 1850 mg to about 2100 mg, about 1850 mg to about 2050 mg, about 1850 mg to about 2000 mg, about 1850 mg to about 1950 mg, about 1850 mg to about 1900 mg, about 1900 mg to about 3600 mg, about 1900 mg to about 3550 mg, about 1900 mg to about 3500 mg, about 1900 mg to about 3450 mg, about 1900 mg to about 3400 mg, about 1900 mg to about 3350 mg, about 1900 mg to about 3300 mg, about 1900 mg to about 3250 mg, about 1900 mg to about 3200 mg, about 1900 mg to about 3150 mg, about 1900 mg to about 3100 mg, about 1900 mg to about 3050 mg, about 1900 mg to about 3000 mg, about 1900 mg to about 2950 mg, about 1900 mg to about 2900 mg, about 1900 mg to about 2850 mg, about 1900 mg to about 2800 mg, about 1900 mg to about 2750 mg, about 1900 mg to about 2700 mg, about 1900 mg to about 2650 mg, about 1900 mg to about 2600 mg, about 1900 mg to about 2550 mg, about 1900 mg to about 2500 mg, about 1900 mg to about 2450 mg, about 1900 mg to about 2400 mg, about 1900 mg to about 2350 mg, about 1900 mg to about 2300 mg, about 1900 mg to about 2250 mg, about 1900 mg to about 2200 mg, about 1900 mg to about 2150 mg, about 1900 mg to about 2100 mg, about 1900 mg to about 2050 mg, about 1900 mg to about 2000 mg, about 1900 mg to about 1950 mg, about 1950 mg to about 3600 mg, about 1950 mg to about 3550 mg, about 1950 mg to about 3500 mg, about 1950 mg to about 3450 mg, about 1950 mg to about 3400 mg, about 1950 mg to about 3350 mg, about 1950 mg to about 3300 mg, about 1950 mg to about 3250 mg, about 1950 mg to about 3200 mg, about 1950 mg to about 3150 mg, about 1950 mg to about 3100 mg, about 1950 mg to about 3050 mg, about 1950 mg to about 3000 mg, about 1950 mg to about 2950 mg, about 1950 mg to about 2900 mg, about 1950 mg to about 2850 mg, about 1950 mg to about 2800 mg, about 1950 mg to about 2750 mg, about 1950 mg to about 2700 mg, about 1950 mg to about 2650 mg, about 1950 mg to about 2600 mg, about 1950 mg to about 2550 mg, about 1950 mg to about 2500 mg, about 1950 mg to about 2450 mg, about 1950 mg to about 2400 mg, about 1950 mg to about 2350 mg, about 1950 mg to about 2300 mg, about 1950 mg to about 2250 mg, about 1950 mg to about 2200 mg, about 1950 mg to about 2150 mg, about 1950 mg to about 2100 mg, about 1950 mg to about 2050 mg, about 1950 mg to about 2000 mg, about 2000 mg to about 3600 mg, about 2000 mg to about 3550 mg, about 2000 mg to about 3500 mg, about 2000 mg to about 3450 mg, about 2000 mg to about 3400 mg, about 2000 mg to about 3350 mg, about 2000 mg to about 3300 mg, about 2000 mg to about 3250 mg, about 2000 mg to about 3200 mg, about 2000 mg to about 3150 mg, about 2000 mg to about 3100 mg, about 2000 mg to about 3050 mg, about 2000 mg to about 3000 mg, about 2000 mg to about 2950 mg, about 2000 mg to about 2900 mg, about 2000 mg to about 2850 mg, about 2000 mg to about 2800 mg, about 2000 mg to about 2750 mg, about 2000 mg to about 2700 mg, about 2000 mg to about 2650 mg, about 2000 mg to about 2600 mg, about 2000 mg to about 2550 mg, about 2000 mg to about 2500 mg, about 2000 mg to about 2450 mg, about 2000 mg to about 2400 mg, about 2000 mg to about 2350 mg, about 2000 mg to about 2300 mg, about 2000 mg to about 2250 mg, about 2000 mg to about 2200 mg, about 2000 mg to about 2150 mg, about 2000 mg to about 2100 mg, about 2000 mg to about 2050 mg, about 2050 mg to about 3600 mg, about 2050 mg to about 3550 mg, about 2050 mg to about 3500 mg, about 2050 mg to about 3450 mg, about 2050 mg to about 3400 mg, about 2050 mg to about 3350 mg, about 2050 mg to about 3300 mg, about 2050 mg to about 3250 mg, about 2050 mg to about 3200 mg, about 2050 mg to about 3150 mg, about 2050 mg to about 3100 mg, about 2050 mg to about 3050 mg, about 2050 mg to about 3000 mg, about 2050 mg to about 2950 mg, about 2050 mg to about 2900 mg, about 2050 mg to about 2850 mg, about 2050 mg to about 2800 mg, about 2050 mg to about 2750 mg, about 2050 mg to about 2700 mg, about 2050 mg to about 2650 mg, about 2050 mg to about 2600 mg, about 2050 mg to about 2550 mg, about 2050 mg to about 2500 mg, about 2050 mg to about 2450 mg, about 2050 mg to about 2400 mg, about 2050 mg to about 2350 mg, about 2050 mg to about 2300 mg, about 2050 mg to about 2250 mg, about 2050 mg to about 2200 mg, about 2050 mg to about 2150 mg, about 2050 mg to about 2100 mg, about 2100 mg to about 3600 mg, about 2100 mg to about 3550 mg, about 2100 mg to about 3500 mg, about 2100 mg to about 3450 mg, about 2100 mg to about 3400 mg, about 2100 mg to about 3350 mg, about 2100 mg to about 3300 mg, about 2100 mg to about 3250 mg, about 2100 mg to about 3200 mg, about 2100 mg to about 3150 mg, about 2100 mg to about 3100 mg, about 2100 mg to about 3050 mg, about 2100 mg to about 3000 mg, about 2100 mg to about 2950 mg, about 2100 mg to about 2900 mg, about 2100 mg to about 2850 mg, about 2100 mg to about 2800 mg, about 2100 mg to about 2750 mg, about 2100 mg to about 2700 mg, about 2100 mg to about 2650 mg, about 2100 mg to about 2600 mg, about 2100 mg to about 2550 mg, about 2100 mg to about 2500 mg, about 2100 mg to about 2450 mg, about 2100 mg to about 2400 mg, about 2100 mg to about 2350 mg, about 2100 mg to about 2300 mg, about 2100 mg to about 2250 mg, about 2100 mg to about 2200 mg, about 2100 mg to about 2150 mg, about 2150 mg to about 3600 mg, about 2150 mg to about 3550 mg, about 2150 mg to about 3500 mg, about 2150 mg to about 3450 mg, about 2150 mg to about 3400 mg, about 2150 mg to about 3350 mg, about 2150 mg to about 3300 mg, about 2150 mg to about 3250 mg, about 2150 mg to about 3200 mg, about 2150 mg to about 3150 mg, about 2150 mg to about 3100 mg, about 2150 mg to about 3050 mg, about 2150 mg to about 3000 mg, about 2150 mg to about 2950 mg, about 2150 mg to about 2900 mg, about 2150 mg to about 2850 mg, about 2150 mg to about 2800 mg, about 2150 mg to about 2750 mg, about 2150 mg to about 2700 mg, about 2150 mg to about 2650 mg, about 2150 mg to about 2600 mg, about 2150 mg to about 2550 mg, about 2150 mg to about 2500 mg, about 2150 mg to about 2450 mg, about 2150 mg to about 2400 mg, about 2150 mg to about 2350 mg, about 2150 mg to about 2300 mg, about 2150 mg to about 2250 mg, about 2150 mg to about 2200 mg, about 2200 mg to about 3600 mg, about 2200 mg to about 3550 mg, about 2200 mg to about 3500 mg, about 2200 mg to about 3450 mg, about 2200 mg to about 3400 mg, about 2200 mg to about 3350 mg, about 2200 mg to about 3300 mg, about 2200 mg to about 3250 mg, about 2200 mg to about 3200 mg, about 2200 mg to about 3150 mg, about 2200 mg to about 3100 mg, about 2200 mg to about 3050 mg, about 2200 mg to about 3000 mg, about 2200 mg to about 2950 mg, about 2200 mg to about 2900 mg, about 2200 mg to about 2850 mg, about 2200 mg to about 2800 mg, about 2200 mg to about 2750 mg, about 2200 mg to about 2700 mg, about 2200 mg to about 2650 mg, about 2200 mg to about 2600 mg, about 2200 mg to about 2550 mg, about 2200 mg to about 2500 mg, about 2200 mg to about 2450 mg, about 2200 mg to about 2400 mg, about 2200 mg to about 2350 mg, about 2200 mg to about 2300 mg, about 2200 mg to about 2250 mg, about 2250 mg to about 3600 mg, about 2250 mg to about 3550 mg, about 2250 mg to about 3500 mg, about 2250 mg to about 3450 mg, about 2250 mg to about 3400 mg, about 2250 mg to about 3350 mg, about 2250 mg to about 3300 mg, about 2250 mg to about 3250 mg, about 2250 mg to about 3200 mg, about 2250 mg to about 3150 mg, about 2250 mg to about 3100 mg, about 2250 mg to about 3050 mg, about 2250 mg to about 3000 mg, about 2250 mg to about 2950 mg, about 2250 mg to about 2900 mg, about 2250 mg to about 2850 mg, about 2250 mg to about 2800 mg, about 2250 mg to about 2750 mg, about 2250 mg to about 2700 mg, about 2250 mg to about 2650 mg, about 2250 mg to about 2600 mg, about 2250 mg to about 2550 mg, about 2250 mg to about 2500 mg, about 2250 mg to about 2450 mg, about 2250 mg to about 2400 mg, about 2250 mg to about 2350 mg, about 2250 mg to about 2300 mg, about 2300 mg to about 3600 mg, about 2300 mg to about 3550 mg, about 2300 mg to about 3500 mg, about 2300 mg to about 3450 mg, about 2300 mg to about 3400 mg, about 2300 mg to about 3350 mg, about 2300 mg to about 3300 mg, about 2300 mg to about 3250 mg, about 2300 mg to about 3200 mg, about 2300 mg to about 3150 mg, about 2300 mg to about 3100 mg, about 2300 mg to about 3050 mg, about 2300 mg to about 3000 mg, about 2300 mg to about 2950 mg, about 2300 mg to about 2900 mg, about 2300 mg to about 2850 mg, about 2300 mg to about 2800 mg, about 2300 mg to about 2750 mg, about 2300 mg to about 2700 mg, about 2300 mg to about 2650 mg, about 2300 mg to about 2600 mg, about 2300 mg to about 2550 mg, about 2300 mg to about 2500 mg, about 2300 mg to about 2450 mg, about 2300 mg to about 2400 mg, about 2300 mg to about 2350 mg, about 2350 mg to about 3600 mg, about 2350 mg to about 3550 mg, about 2350 mg to about 3500 mg, about 2350 mg to about 3450 mg, about 2350 mg to about 3400 mg, about 2350 mg to about 3350 mg, about 2350 mg to about 3300 mg, about 2350 mg to about 3250 mg, about 2350 mg to about 3200 mg, about 2350 mg to about 3150 mg, about 2350 mg to about 3100 mg, about 2350 mg to about 3050 mg, about 2350 mg to about 3000 mg, about 2350 mg to about 2950 mg, about 2350 mg to about 2900 mg, about 2350 mg to about 2850 mg, about 2350 mg to about 2800 mg, about 2350 mg to about 2750 mg, about 2350 mg to about 2700 mg, about 2350 mg to about 2650 mg, about 2350 mg to about 2600 mg, about 2350 mg to about 2550 mg, about 2350 mg to about 2500 mg, about 2350 mg to about 2450 mg, about 2350 mg to about 2400 mg, about 2400 mg to about 3600 mg, about 2400 mg to about 3550 mg, about 2400 mg to about 3500 mg, about 2400 mg to about 3450 mg, about 2400 mg to about 3400 mg, about 2400 mg to about 3350 mg, about 2400 mg to about 3300 mg, about 2400 mg to about 3250 mg, about 2400 mg to about 3200 mg, about 2400 mg to about 3150 mg, about 2400 mg to about 3100 mg, about 2400 mg to about 3050 mg, about 2400 mg to about 3000 mg, about 2400 mg to about 2950 mg, about 2400 mg to about 2900 mg, about 2400 mg to about 2850 mg, about 2400 mg to about 2800 mg, about 2400 mg to about 2750 mg, about 2400 mg to about 2700 mg, about 2400 mg to about 2650 mg, about 2400 mg to about 2600 mg, about 2400 mg to about 2550 mg, about 2400 mg to about 2500 mg, about 2400 mg to about 2450 mg, about 2450 mg to about 3600 mg, about 2450 mg to about 3550 mg, about 2450 mg to about 3500 mg, about 2450 mg to about 3450 mg, about 2450 mg to about 3400 mg, about 2450 mg to about 3350 mg, about 2450 mg to about 3300 mg, about 2450 mg to about 3250 mg, about 2450 mg to about 3200 mg, about 2450 mg to about 3150 mg, about 2450 mg to about 3100 mg, about 2450 mg to about 3050 mg, about 2450 mg to about 3000 mg, about 2450 mg to about 2950 mg, about 2450 mg to about 2900 mg, about 2450 mg to about 2850 mg, about 2450 mg to about 2800 mg, about 2450 mg to about 2750 mg, about 2450 mg to about 2700 mg, about 2450 mg to about 2650 mg, about 2450 mg to about 2600 mg, about 2450 mg to about 2550 mg, about 2450 mg to about 2500 mg, about 2500 mg to about 3600 mg, about 2500 mg to about 3550 mg, about 2500 mg to about 3500 mg, about 2500 mg to about 3450 mg, about 2500 mg to about 3400 mg, about 2500 mg to about 3350 mg, about 2500 mg to about 3300 mg, about 2500 mg to about 3250 mg, about 2500 mg to about 3200 mg, about 2500 mg to about 3150 mg, about 2500 mg to about 3100 mg, about 2500 mg to about 3050 mg, about 2500 mg to about 3000 mg, about 2500 mg to about 2950 mg, about 2500 mg to about 2900 mg, about 2500 mg to about 2850 mg, about 2500 mg to about 2800 mg, about 2500 mg to about 2750 mg, about 2500 mg to about 2700 mg, about 2500 mg to about 2650 mg, about 2500 mg to about 2600 mg, about 2500 mg to about 2550 mg, about 2550 mg to about 3600 mg, about 2550 mg to about 3550 mg, about 2550 mg to about 3500 mg, about 2550 mg to about 3450 mg, about 2550 mg to about 3400 mg, about 2550 mg to about 3350 mg, about 2550 mg to about 3300 mg, about 2550 mg to about 3250 mg, about 2550 mg to about 3200 mg, about 2550 mg to about 3150 mg, about 2550 mg to about 3100 mg, about 2550 mg to about 3050 mg, about 2550 mg to about 3000 mg, about 2550 mg to about 2950 mg, about 2550 mg to about 2900 mg, about 2550 mg to about 2850 mg, about 2550 mg to about 2800 mg, about 2550 mg to about 2750 mg, about 2550 mg to about 2700 mg, about 2550 mg to about 2650 mg, about 2550 mg to about 2600 mg, about 2600 mg to about 3600 mg, about 2600 mg to about 3550 mg, about 2600 mg to about 3500 mg, about 2600 mg to about 3450 mg, about 2600 mg to about 3400 mg, about 2600 mg to about 3350 mg, about 2600 mg to about 3300 mg, about 2600 mg to about 3250 mg, about 2600 mg to about 3200 mg, about 2600 mg to about 3150 mg, about 2600 mg to about 3100 mg, about 2600 mg to about 3050 mg, about 2600 mg to about 3000 mg, about 2600 mg to about 2950 mg, about 2600 mg to about 2900 mg, about 2600 mg to about 2850 mg, about 2600 mg to about 2800 mg, about 2600 mg to about 2750 mg, about 2600 mg to about 2700 mg, about 2600 mg to about 2650 mg, about 2650 mg to about 3600 mg, about 2650 mg to about 3550 mg, about 2650 mg to about 3500 mg, about 2650 mg to about 3450 mg, about 2650 mg to about 3400 mg, about 2650 mg to about 3350 mg, about 2650 mg to about 3300 mg, about 2650 mg to about 3250 mg, about 2650 mg to about 3200 mg, about 2650 mg to about 3150 mg, about 2650 mg to about 3100 mg, about 2650 mg to about 3050 mg, about 2650 mg to about 3000 mg, about 2650 mg to about 2950 mg, about 2650 mg to about 2900 mg, about 2650 mg to about 2850 mg, about 2650 mg to about 2800 mg, about 2650 mg to about 2750 mg, about 2650 mg to about 2700 mg, about 2700 mg to about 3600 mg, about 2700 mg to about 3550 mg, about 2700 mg to about 3500 mg, about 2700 mg to about 3450 mg, about 2700 mg to about 3400 mg, about 2700 mg to about 3350 mg, about 2700 mg to about 3300 mg, about 2700 mg to about 3250 mg, about 2700 mg to about 3200 mg, about 2700 mg to about 3150 mg, about 2700 mg to about 3100 mg, about 2700 mg to about 3050 mg, about 2700 mg to about 3000 mg, about 2700 mg to about 2950 mg, about 2700 mg to about 2900 mg, about 2700 mg to about 2850 mg, about 2700 mg to about 2800 mg, about 2700 mg to about 2750 mg, about 2750 mg to about 3600 mg, about 2750 mg to about 3550 mg, about 2750 mg to about 3500 mg, about 2750 mg to about 3450 mg, about 2750 mg to about 3400 mg, about 2750 mg to about 3350 mg, about 2750 mg to about 3300 mg, about 2750 mg to about 3250 mg, about 2750 mg to about 3200 mg, about 2750 mg to about 3150 mg, about 2750 mg to about 3100 mg, about 2750 mg to about 3050 mg, about 2750 mg to about 3000 mg, about 2750 mg to about 2950 mg, about 2750 mg to about 2900 mg, about 2750 mg to about 2850 mg, about 2750 mg to about 2800 mg, about 2800 mg to about 3600 mg, about 2800 mg to about 3550 mg, about 2800 mg to about 3500 mg, about 2800 mg to about 3450 mg, about 2800 mg to about 3400 mg, about 2800 mg to about 3350 mg, about 2800 mg to about 3300 mg, about 2800 mg to about 3250 mg, about 2800 mg to about 3200 mg, about 2800 mg to about 3150 mg, about 2800 mg to about 3100 mg, about 2800 mg to about 3050 mg, about 2800 mg to about 3000 mg, about 2800 mg to about 2950 mg, about 2800 mg to about 2900 mg, about 2800 mg to about 2850 mg, about 2850 mg to about 3600 mg, about 2850 mg to about 3550 mg, about 2850 mg to about 3500 mg, about 2850 mg to about 3450 mg, about 2850 mg to about 3400 mg, about 2850 mg to about 3350 mg, about 2850 mg to about 3300 mg, about 2850 mg to about 3250 mg, about 2850 mg to about 3200 mg, about 2850 mg to about 3150 mg, about 2850 mg to about 3100 mg, about 2850 mg to about 3050 mg, about 2850 mg to about 3000 mg, about 2850 mg to about 2950 mg, about 2850 mg to about 2900 mg, about 2900 mg to about 3600 mg, about 2900 mg to about 3550 mg, about 2900 mg to about 3500 mg, about 2900 mg to about 3450 mg, about 2900 mg to about 3400 mg, about 2900 mg to about 3350 mg, about 2900 mg to about 3300 mg, about 2900 mg to about 3250 mg, about 2900 mg to about 3200 mg, about 2900 mg to about 3150 mg, about 2900 mg to about 3100 mg, about 2900 mg to about 3050 mg, about 2900 mg to about 3000 mg, about 2900 mg to about 2950 mg, about 2950 mg to about 3600 mg, about 2950 mg to about 3550 mg, about 2950 mg to about 3500 mg, about 2950 mg to about 3450 mg, about 2950 mg to about 3400 mg, about 2950 mg to about 3350 mg, about 2950 mg to about 3300 mg, about 2950 mg to about 3250 mg, about 2950 mg to about 3200 mg, about 2950 mg to about 3150 mg, about 2950 mg to about 3100 mg, about 2950 mg to about 3050 mg, about 2950 mg to about 3000 mg, about 3000 mg to about 3600 mg, about 3000 mg to about 3550 mg, about 3000 mg to about 3500 mg, about 3000 mg to about 3450 mg, about 3000 mg to about 3400 mg, about 3000 mg to about 3350 mg, about 3000 mg to about 3300 mg, about 3000 mg to about 3250 mg, about 3000 mg to about 3200 mg, about 3000 mg to about 3150 mg, about 3000 mg to about 3100 mg, about 3000 mg to about 3050 mg, about 3050 mg to about 3600 mg, about 3050 mg to about 3550 mg, about 3050 mg to about 3500 mg, about 3050 mg to about 3450 mg, about 3050 mg to about 3400 mg, about 3050 mg to about 3350 mg, about 3050 mg to about 3300 mg, about 3050 mg to about 3250 mg, about 3050 mg to about 3200 mg, about 3050 mg to about 3150 mg, about 3050 mg to about 3100 mg, about 3100 mg to about 3600 mg, about 3100 mg to about 3550 mg, about 3100 mg to about 3500 mg, about 3100 mg to about 3450 mg, about 3100 mg to about 3400 mg, about 3100 mg to about 3350 mg, about 3100 mg to about 3300 mg, about 3100 mg to about 3250 mg, about 3100 mg to about 3200 mg, about 3100 mg to about 3150 mg, about 3150 mg to about 3600 mg, about 3150 mg to about 3550 mg, about 3150 mg to about 3500 mg, about 3150 mg to about 3450 mg, about 3150 mg to about 3400 mg, about 3150 mg to about 3350 mg, about 3150 mg to about 3300 mg, about 3150 mg to about 3250 mg, about 3150 mg to about 3200 mg, about 3200 mg to about 3600 mg, about 3200 mg to about 3550 mg, about 3200 mg to about 3500 mg, about 3200 mg to about 3450 mg, about 3200 mg to about 3400 mg, about 3200 mg to about 3350 mg, about 3200 mg to about 3300 mg, about 3200 mg to about 3250 mg, about 3250 mg to about 3600 mg, about 3250 mg to about 3550 mg, about 3250 mg to about 3500 mg, about 3250 mg to about 3450 mg, about 3250 mg to about 3400 mg, about 3250 mg to about 3350 mg, about 3250 mg to about 3300 mg, about 3300 mg to about 3600 mg, about 3300 mg to about 3550 mg, about 3300 mg to about 3500 mg, about 3300 mg to about 3450 mg, about 3300 mg to about 3400 mg, about 3300 mg to about 3350 mg, about 3350 mg to about 3600 mg, about 3350 mg to about 3550 mg, about 3350 mg to about 3500 mg, about 3350 mg to about 3450 mg, about 3350 mg to about 3400 mg, about 3400 mg to about 3600 mg, about 3400 mg to about 3550 mg, about 3400 mg to about 3500 mg, about 3400 mg to about 3450 mg, about 3450 mg to about 3600 mg, about 3450 mg to about 3550 mg, about 3450 mg to about 3500 mg, about 3500 mg to about 3600 mg, about 3500 mg to about 3550 mg, or about 3550 mg to about 3600 mg.

In one aspect, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of from about 1800 mg to about 4000 mg (e.g., about 3600 mg). The C1D1 may be administered, for example, IV or SC. In some aspects, the C1D1 is administered IV. In other aspects, the C1D1 is administered SC (e.g., by a pump (e.g., by a patch pump)).

In another aspect, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of from about 1800 mg to about 4000 mg (e.g., about 3600 mg). In some aspects, the C1D1 is administered IV. In other aspects, the C1D1 is administered SC (e.g., by a pump (e.g., by a patch pump)).

In another aspect, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of from about 1800 mg to about 4000 mg (e.g., about 3600 mg). In some aspects, the C1D1 is administered IV. In other aspects, the C1D1 is administered SC (e.g., by a pump (e.g., by a patch pump)).

For example, in any of the preceding aspects, the first dose (C1D1) of the anti-tryptase antibody, and/or any additional doses of the anti-tryptase antibody, may be about 1800 mg to about 4000 mg, about 1800 mg to about 3900 mg, about 1800 mg to about 3800 mg, about 1800 mg to about 3700 mg, about 1800 mg to about 3600 mg, about 1800 mg to about 3500 mg, about 1800 mg to about 3400 mg, about 1800 mg to about 3300 mg, about 1800 mg to about 3200 mg, about 1800 mg to about 3100 mg, about 1800 mg to about 3000 mg, about 1800 mg to about 2900 mg, about 1800 mg to about 2800 mg, about 1800 mg to about 2700 mg, about 1800 mg to about 2600 mg, about 1800 mg to about 2500 mg, about 1800 mg to about 2400 mg, about 1800 mg to about 2300 mg, about 1800 mg to about 2200 mg, about 1800 mg to about 2100 mg, about 1800 mg to about 2000 mg, about 1800 mg to about 1900 mg, about 1900 mg to about 4000 mg, about 1900 mg to about 3900 mg, about 1900 mg to about 3800 mg, about 1900 mg to about 3700 mg, about 1900 mg to about 3600 mg, about 1900 mg to about 3500 mg, about 1900 mg to about 3400 mg, about 1900 mg to about 3300 mg, about 1900 mg to about 3200 mg, about 1900 mg to about 3100 mg, about 1900 mg to about 3000 mg, about 1900 mg to about 2900 mg, about 1900 mg to about 2800 mg, about 1900 mg to about 2700 mg, about 1900 mg to about 2600 mg, about 1900 mg to about 2500 mg, about 1900 mg to about 2400 mg, about 1900 mg to about 2300 mg, about 1900 mg to about 2200 mg, about 1900 mg to about 2100 mg, about 1900 mg to about 2000 mg, about 2000 mg to about 4000 mg, about 2000 mg to about 3900 mg, about 2000 mg to about 3800 mg, about 2000 mg to about 3700 mg, about 2000 mg to about 3600 mg, about 2000 mg to about 3500 mg, about 2000 mg to about 3400 mg, about 2000 mg to about 3300 mg, about 2000 mg to about 3200 mg, about 2000 mg to about 3100 mg, about 2000 mg to about 3000 mg, about 2000 mg to about 2900 mg, about 2000 mg to about 2800 mg, about 2000 mg to about 2700 mg, about 2000 mg to about 2600 mg, about 2000 mg to about 2500 mg, about 2000 mg to about 2400 mg, about 2000 mg to about 2300 mg, about 2000 mg to about 2200 mg, about 2000 mg to about 2100 mg, about 2100 mg to about 4000 mg, about 2100 mg to about 3900 mg, about 2100 mg to about 3800 mg, about 2100 mg to about 3700 mg, about 2100 mg to about 3600 mg, about 2100 mg to about 3500 mg, about 2100 mg to about 3400 mg, about 2100 mg to about 3300 mg, about 2100 mg to about 3200 mg, about 2100 mg to about 3100 mg, about 2100 mg to about 3000 mg, about 2100 mg to about 2900 mg, about 2100 mg to about 2800 mg, about 2100 mg to about 2700 mg, about 2100 mg to about 2600 mg, about 2100 mg to about 2500 mg, about 2100 mg to about 2400 mg, about 2100 mg to about 2300 mg, about 2100 mg to about 2200 mg, about 2200 mg to about 4000 mg, about 2200 mg to about 3900 mg, about 2200 mg to about 3800 mg, about 2200 mg to about 3700 mg, about 2200 mg to about 3600 mg, about 2200 mg to about 3500 mg, about 2200 mg to about 3400 mg, about 2200 mg to about 3300 mg, about 2200 mg to about 3200 mg, about 2200 mg to about 3100 mg, about 2200 mg to about 3000 mg, about 2200 mg to about 2900 mg, about 2200 mg to about 2800 mg, about 2200 mg to about 2700 mg, about 2200 mg to about 2600 mg, about 2200 mg to about 2500 mg, about 2200 mg to about 2400 mg, about 2200 mg to about 2300 mg, about 2300 mg to about 4000 mg, about 2300 mg to about 3900 mg, about 2300 mg to about 3800 mg, about 2300 mg to about 3700 mg, about 2300 mg to about 3600 mg, about 2300 mg to about 3500 mg, about 2300 mg to about 3400 mg, about 2300 mg to about 3300 mg, about 2300 mg to about 3200 mg, about 2300 mg to about 3100 mg, about 2300 mg to about 3000 mg, about 2300 mg to about 2900 mg, about 2300 mg to about 2800 mg, about 2300 mg to about 2700 mg, about 2300 mg to about 2600 mg, about 2300 mg to about 2500 mg, about 2300 mg to about 2400 mg, about 2400 mg to about 4000 mg, about 2400 mg to about 3900 mg, about 2400 mg to about 3800 mg, about 2400 mg to about 3700 mg, about 2400 mg to about 3600 mg, about 2400 mg to about 3500 mg, about 2400 mg to about 3400 mg, about 2400 mg to about 3300 mg, about 2400 mg to about 3200 mg, about 2400 mg to about 3100 mg, about 2400 mg to about 3000 mg, about 2400 mg to about 2900 mg, about 2400 mg to about 2800 mg, about 2400 mg to about 2700 mg, about 2400 mg to about 2600 mg, about 2400 mg to about 2500 mg, about 2500 mg to about 4000 mg, about 2500 mg to about 3900 mg, about 2500 mg to about 3800 mg, about 2500 mg to about 3700 mg, about 2500 mg to about 3600 mg, about 2500 mg to about 3500 mg, about 2500 mg to about 3400 mg, about 2500 mg to about 3300 mg, about 2500 mg to about 3200 mg, about 2500 mg to about 3100 mg, about 2500 mg to about 3000 mg, about 2500 mg to about 2900 mg, about 2500 mg to about 2800 mg, about 2500 mg to about 2700 mg, about 2500 mg to about 2600 mg, about 2600 mg to about 4000 mg, about 2600 mg to about 3900 mg, about 2600 mg to about 3800 mg, about 2600 mg to about 3700 mg, about 2600 mg to about 3600 mg, about 2600 mg to about 3500 mg, about 2600 mg to about 3400 mg, about 2600 mg to about 3300 mg, about 2600 mg to about 3200 mg, about 2600 mg to about 3100 mg, about 2600 mg to about 3000 mg, about 2600 mg to about 2900 mg, about 2600 mg to about 2800 mg, about 2600 mg to about 2700 mg, about 2700 mg to about 4000 mg, about 2700 mg to about 3900 mg, about 2700 mg to about 3800 mg, about 2700 mg to about 3700 mg, about 2700 mg to about 3600 mg, about 2700 mg to about 3500 mg, about 2700 mg to about 3400 mg, about 2700 mg to about 3300 mg, about 2700 mg to about 3200 mg, about 2700 mg to about 3100 mg, about 2700 mg to about 3000 mg, about 2700 mg to about 2900 mg, about 2700 mg to about 2800 mg, about 2800 mg to about 4000 mg, about 2800 mg to about 3900 mg, about 2800 mg to about 3800 mg, about 2800 mg to about 3700 mg, about 2800 mg to about 3600 mg, about 2800 mg to about 3500 mg, about 2800 mg to about 3400 mg, about 2800 mg to about 3300 mg, about 2800 mg to about 3200 mg, about 2800 mg to about 3100 mg, about 2800 mg to about 3000 mg, about 2800 mg to about 2900 mg, about 2900 mg to about 4000 mg, about 2900 mg to about 3900 mg, about 2900 mg to about 3800 mg, about 2900 mg to about 3700 mg, about 2900 mg to about 3600 mg, about 2900 mg to about 3500 mg, about 2900 mg to about 3400 mg, about 2900 mg to about 3300 mg, about 2900 mg to about 3200 mg, about 2900 mg to about 3100 mg, about 2900 mg to about 3000 mg, about 3000 mg to about 4000 mg, about 3000 mg to about 3900 mg, about 3000 mg to about 3800 mg, about 3000 mg to about 3700 mg, about 3000 mg to about 3600 mg, about 3000 mg to about 3500 mg, about 3000 mg to about 3400 mg, about 3000 mg to about 3300 mg, about 3000 mg to about 3200 mg, about 3000 mg to about 3100 mg, about 3100 mg to about 4000 mg, about 3100 mg to about 3900 mg, about 3100 mg to about 3800 mg, about 3100 mg to about 3700 mg, about 3100 mg to about 3600 mg, about 3100 mg to about 3500 mg, about 3100 mg to about 3400 mg, about 3100 mg to about 3300 mg, about 3100 mg to about 3200 mg, about 3200 mg to about 4000 mg, about 3200 mg to about 3900 mg, about 3200 mg to about 3800 mg, about 3200 mg to about 3700 mg, about 3200 mg to about 3600 mg, about 3200 mg to about 3500 mg, about 3200 mg to about 3400 mg, about 3200 mg to about 3300 mg, about 3300 mg to about 4000 mg, about 3300 mg to about 3900 mg, about 3300 mg to about 3800 mg, about 3300 mg to about 3700 mg, about 3300 mg to about 3600 mg, about 3300 mg to about 3500 mg, about 3300 mg to about 3400 mg, about 3400 mg to about 4000 mg, about 3400 mg to about 3900 mg, about 3400 mg to about 3800 mg, about 3400 mg to about 3700 mg, about 3400 mg to about 3600 mg, about 3400 mg to about 3500 mg, about 3500 mg to about 4000 mg, about 3500 mg to about 3900 mg, about 3500 mg to about 3800 mg, about 3500 mg to about 3700 mg, about 3500 mg to about 3600 mg, about 3600 mg to about 4000 mg, about 3600 mg to about 3900 mg, about 3600 mg to about 3800 mg, about 3600 mg to about 3700 mg, about 3700 mg to about 4000 mg, about 3700 mg to about 3900 mg, about 3700 mg to about 3800 mg, about 3800 mg to about 4000 mg, about 3800 mg to about 3900 mg, or about 3900 mg to about 4000 mg.

In one aspect, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody selected from 300 mg, 450 mg, 600 mg, 750 mg, 900 mg, 1350 mg, 1800 mg, or 3600 mg. The C1D1 may be administered, for example, intravenously (IV) or subcutaneously (SC) (e.g., by a pump (e.g., by a patch pump)).

In some aspects, any of the doses disclosed herein may be administered IV. Any suitable approach for IV administration may be used, including injection (e.g., a bolus injection) or infusion. In some examples, the anti-tryptase antibody may be administered IV by infusion. For example, the IV infusion may use pressure supplied by gravity (e.g., a drip) or using a pump (e.g., an infusion pump). In some examples, the IV infusion may be continuous or intermittent. In some examples, a central venous catheter, a peripheral venous catheter, a peripherally inserted central catheter (PICC), a midline catheter, or an implantable port may be used for IV administration. In some examples, the anti-tryptase antibody may be administered IV using a pump. Any suitable pump may be used for IV administration, for example, an infusion pump (e.g., an ambulatory infusion pump or a stationary infusion pump), a syringe pump, a patch pump, or a large volume pump (LVP).

In other aspects, any of the doses disclosed herein may be administered SC. Any suitable approach for SC administration may be used, including injection (e.g., a bolus injection) or infusion. For example, the anti-tryptase antibody may be administered SC using a pump (e.g., a patch pump, a syringe pump (e.g., a syringe pump with an infusion set), or an infusion pump (e.g., an ambulatory infusion pump or a stationary infusion pump)), a pre-filled syringe, a pen injector, or an autoinjector.

For example, in any of the methods or uses disclosed herein, the anti-tryptase antibody may be administered SC using a pump. In some examples, a pump may be used for patient or health care provider (HCP) convenience, an improved safety profile (e.g., in terms of a drug's mechanism of action or the risk of IV-related infection), and/or for a combination therapy. Any suitable pump may be used, e.g., a patch pump, a syringe pump (e.g., a syringe pump with an infusion set), an infusion pump (e.g., an ambulatory infusion pump or a stationary infusion pump), or an LVP. In particular examples, the anti-tryptase antibody may be administered SC using a patch pump. In some examples, the pump (e.g., the patch pump) may be a wearable or on-body pump (e.g., a wearable or on-body patch pump), for example, an Enable ENFUSE® on-body infusor or a West SMARTDOSE® wearable injector (e.g., a West SMARTDOSE® 10 wearable injector). In other examples, the anti-tryptase antibody may be administered SC using a syringe pump (e.g., a syringe pump with an infusion set).

For example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of 300 mg. In some aspects, the C1D1 is administered IV. In other aspects, the C1D1 is administered SC (e.g., by a pump (e.g., by a patch pump)).

In another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of 450 mg. In some aspects, the C1D1 is administered IV. In other aspects, the C1D1 is administered SC (e.g., by a pump (e.g., by a patch pump)).

In another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of 600 mg. In some aspects, the C1D1 is administered IV. In other aspects, the C1D1 is administered SC (e.g., by a pump (e.g., by a patch pump)).

In yet another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of 750 mg. In some aspects, the C1D1 is administered IV. In other aspects, the C1D1 is administered SC (e.g., by a pump (e.g., by a patch pump)).

In a further example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of 900 mg. In some aspects, the C1D1 is administered IV. In other aspects, the C1D1 is administered SC (e.g., by a pump (e.g., by a patch pump)).

In yet a further example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of 1350 mg. In some aspects, the C1D1 is administered IV. In other aspects, the C1D1 is administered SC (e.g., by a pump (e.g., by a patch pump)).

In a still further example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of 1800 mg. In some aspects, the C1D1 is administered IV. In other aspects, the C1D1 is administered SC (e.g., by a pump (e.g., by a patch pump)).

In another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1) of the anti-tryptase antibody of 3600 mg. In some aspects, the C1D1 is administered IV. In other aspects, the C1D1 is administered SC (e.g., by a pump (e.g., by a patch pump)).

In another aspect, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody selected from 300 mg, 450 mg, 600 mg, 750 mg, 900 mg, 1350 mg, 1800 mg, or 3600 mg. In some aspects, the C1D1 is administered IV. In other aspects, the C1D1 is administered SC (e.g., by a pump (e.g., by a patch pump)).

For example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of 300 mg. In some aspects, the C1D1 is administered IV. In other aspects, the C1D1 is administered SC (e.g., by a pump (e.g., by a patch pump)).

In another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of 450 mg. In some aspects, the C1D1 is administered IV. In other aspects, the C1D1 is administered SC (e.g., by a pump (e.g., by a patch pump)).

In another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of 600 mg. In some aspects, the C1D1 is administered IV. In other aspects, the C1D1 is administered SC (e.g., by a pump (e.g., by a patch pump)).

In yet another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of 750 mg. In some aspects, the C1D1 is administered IV. In other aspects, the C1D1 is administered SC (e.g., by a pump (e.g., by a patch pump)).

In a further example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of 900 mg. In some aspects, the C1D1 is administered IV. In other aspects, the C1D1 is administered SC (e.g., by a pump (e.g., by a patch pump)).

In yet a further example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of 1350 mg. In some aspects, the C1D1 is administered IV. In other aspects, the C1D1 is administered SC (e.g., by a pump (e.g., by a patch pump)).

In a still further example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of 1800 mg. In some aspects, the C1D1 is administered IV. In other aspects, the C1D1 is administered SC (e.g., by a pump (e.g., by a patch pump)).

In another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of 3600 mg. In some aspects, the C1D1 is administered IV. In other aspects, the C1D1 is administered SC (e.g., by a pump (e.g., by a patch pump)).

In another aspect, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody selected from 300 mg, 450 mg, 600 mg, 750 mg, 900 mg, 1350 mg, 1800 mg, or 3600 mg. In some aspects, the C1D1 is administered IV. In other aspects, the C1D1 is administered SC (e.g., by a pump (e.g., by a patch pump)).

For example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of 300 mg. In some aspects, the C1D1 is administered IV. In other aspects, the C1D1 is administered SC (e.g., by a pump (e.g., by a patch pump)).

In another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of 450 mg. In some aspects, the C1D1 is administered IV. In other aspects, the C1D1 is administered SC (e.g., by a pump (e.g., by a patch pump)).

In another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of 600 mg. In some aspects, the C1D1 is administered IV. In other aspects, the C1D1 is administered SC (e.g., by a pump (e.g., by a patch pump)).

In yet another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of 750 mg. In some aspects, the C1D1 is administered IV. In other aspects, the C1D1 is administered SC (e.g., by a pump (e.g., by a patch pump)).

In a further example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of 900 mg. In some aspects, the C1D1 is administered IV. In other aspects, the C1D1 is administered SC (e.g., by a pump (e.g., by a patch pump)).

In yet a further example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of 1350 mg. In some aspects, the C1D1 is administered IV. In other aspects, the C1D1 is administered SC (e.g., by a pump (e.g., by a patch pump)).

In a still further example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of 1800 mg. In some aspects, the C1D1 is administered IV. In other aspects, the C1D1 is administered SC (e.g., by a pump (e.g., by a patch pump)).

In another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1) of the anti-tryptase antibody of 3600 mg. In some aspects, the C1D1 is administered IV. In other aspects, the C1D1 is administered SC (e.g., by a pump (e.g., by a patch pump)).

In any of the aspects disclosed herein, the dosing cycle may further include one or more additional doses of the anti-tryptase antibody. The dosing cycle may include any suitable number of additional doses (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more additional doses) of the anti-tryptase antibody. For example, in some aspects, the dosing cycle may include a second dose (C1D2). In another example, in some aspects, the dosing cycle may include a C1D2 and a third dose (C1D3). The one or more additional doses may be equal to or unequal to the C1D1. For example, in some aspects, the dosing cycle includes a second dose (C1D2) and a third dose (C1D3) of the anti-tryptase antibody, wherein the C1D2 and the C1D3 are each equal to the C1D1. The one or more additional doses may be administered by any suitable administration route, e.g., IV or SC (e.g., by a pump (e.g., by a patch pump)).

For example, in one aspect, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are selected from 300 mg, 450 mg, 600 mg, 750 mg, 900 mg, 1350 mg, 1800 mg, or 3600 mg. In some aspects, the C1D1, the C1D2, and the C1D3 are administered IV. In other aspects, the C1D1, the C1D2, and the C1D3 are administered SC (e.g., by a pump (e.g., by a patch pump)).

For example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 300 mg. In some aspects, the C1D1, the C1D2, and the C1D3 are administered IV. In other aspects, the C1D1, the C1D2, and the C1D3 are administered SC (e.g., by a pump (e.g., by a patch pump)).

In another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 450 mg. In some aspects, the C1D1, the C1D2, and the C1D3 are administered IV. In other aspects, the C1D1, the C1D2, and the C1D3 are administered SC (e.g., by a pump (e.g., by a patch pump)).

In another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 600 mg. In some aspects, the C1D1, the C1D2, and the C1D3 are administered IV. In other aspects, the C1D1, the C1D2, and the C1D3 are administered SC (e.g., by a pump (e.g., by a patch pump)).

In yet another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 750 mg. In some aspects, the C1D1, the C1D2, and the C1D3 are administered IV. In other aspects, the C1D1, the C1D2, and the C1D3 are administered SC (e.g., by a pump (e.g., by a patch pump)).

In a further example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 900 mg. In some aspects, the C1D1, the C1D2, and the C1D3 are administered IV. In other aspects, the C1D1, the C1D2, and the C1D3 are administered SC (e.g., by a pump (e.g., by a patch pump)).

In yet a further example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 1350 mg. In some aspects, the C1D1, the C1D2, and the C1D3 are administered IV. In other aspects, the C1D1, the C1D2, and the C1D3 are administered SC (e.g., by a pump (e.g., by a patch pump)).

In a still further example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 1800 mg. In some aspects, the C1D1, the C1D2, and the C1D3 are administered IV. In other aspects, the C1D1, the C1D2, and the C1D3 are administered SC (e.g., by a pump (e.g., by a patch pump)).

In another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 3600 mg. In some aspects, the C1D1, the C1D2, and the C1D3 are administered IV. In other aspects, the C1D1, the C1D2, and the C1D3 are administered SC (e.g., by a pump (e.g., by a patch pump)).

In another aspect, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are selected from 300 mg, 450 mg, 600 mg, 750 mg, 900 mg, 1350 mg, 1800 mg, or 3600 mg. In some aspects, the C1D1, the C1D2, and the C1D3 are administered IV. In other aspects, the C1D1, the C1D2, and the C1D3 are administered SC (e.g., by a pump (e.g., by a patch pump)).

For example, provided herein is an anti-tryptase antibody (e.g., an anti-tryptase beta antibody) for use in treating a patient having CSU, wherein the anti-tryptase antibody is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 300 mg. In some aspects, the C1D1, the C1D2, and the C1D3 are administered IV. In other aspects, the C1D1, the C1D2, and the C1D3 are administered SC (e.g., by a pump (e.g., by a patch pump)).

In another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 450 mg. In some aspects, the C1D1, the C1D2, and the C1D3 are administered IV. In other aspects, the C1D1, the C1D2, and the C1D3 are administered SC (e.g., by a pump (e.g., by a patch pump)).

In another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 600 mg. In some aspects, the C1D1, the C1D2, and the C1D3 are administered IV. In other aspects, the C1D1, the C1D2, and the C1D3 are administered SC (e.g., by a pump (e.g., by a patch pump)).

In yet another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1) of the anti-tryptase antibody of 750 mg. In some aspects, the C1D1, the C1D2, and the C1D3 are administered IV. In other aspects, the C1D1, the C1D2, and the C1D3 are administered SC (e.g., by a pump (e.g., by a patch pump)).

In a further example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 900 mg. In some aspects, the C1D1, the C1D2, and the C1D3 are administered IV. In other aspects, the C1D1, the C1D2, and the C1D3 are administered SC (e.g., by a pump (e.g., by a patch pump)).

In yet a further example, provided herein is an anti-tryptase antibody (e.g., an anti-tryptase beta antibody) antibody for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 1350 mg. In some aspects, the C1D1, the C1D2, and the C1D3 are administered IV. In other aspects, the C1D1, the C1D2, and the C1D3 are administered SC (e.g., by a pump (e.g., by a patch pump)).

In a still further example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 1800 mg. In some aspects, the C1D1, the C1D2, and the C1D3 are administered IV. In other aspects, the C1D1, the C1D2, and the C1D3 are administered SC (e.g., by a pump (e.g., by a patch pump)).

In another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 3600 mg. In some aspects, the C1D1, the C1D2, and the C1D3 are administered IV. In other aspects, the C1D1, the C1D2, and the C1D3 are administered SC (e.g., by a pump (e.g., by a patch pump)).

In another aspect, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are selected from 300 mg, 450 mg, 600 mg, 750 mg, 900 mg, 1350 mg, 1800 mg, or 3600 mg. In some aspects, the C1D1, the C1D2, and the C1D3 are administered IV. In other aspects, the C1D1, the C1D2, and the C1D3 are administered SC (e.g., by a pump (e.g., by a patch pump)).

For example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 300 mg. In some aspects, the C1D1, the C1D2, and the C1D3 are administered IV. In other aspects, the C1D1, the C1D2, and the C1D3 are administered SC (e.g., by a pump (e.g., by a patch pump)).

In another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 450 mg. In some aspects, the C1D1, the C1D2, and the C1D3 are administered IV. In other aspects, the C1D1, the C1D2, and the C1D3 are administered SC (e.g., by a pump (e.g., by a patch pump)).

In another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 600 mg. In some aspects, the C1D1, the C1D2, and the C1D3 are administered IV. In other aspects, the C1D1, the C1D2, and the C1D3 are administered SC (e.g., by a pump (e.g., by a patch pump)).

In yet another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 750 mg. In some aspects, the C1D1, the C1D2, and the C1D3 are administered IV. In other aspects, the C1D1, the C1D2, and the C1D3 are administered SC (e.g., by a pump (e.g., by a patch pump)).

In a further example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 900 mg. In some aspects, the C1D1, the C1D2, and the C1D3 are administered IV. In other aspects, the C1D1, the C1D2, and the C1D3 are administered SC (e.g., by a pump (e.g., by a patch pump)).

In yet a further example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 1350 mg. In some aspects, the C1D1, the C1D2, and the C1D3 are administered IV. In other aspects, the C1D1, the C1D2, and the C1D3 are administered SC (e.g., by a pump (e.g., by a patch pump)).

In a still further example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 1800 mg. In some aspects, the C1D1, the C1D2, and the C1D3 are administered IV. In other aspects, the C1D1, the C1D2, and the C1D3 are administered SC (e.g., by a pump (e.g., by a patch pump)).

In another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 3600 mg. In some aspects, the C1D1, the C1D2, and the C1D3 are administered IV. In other aspects, the C1D1, the C1D2, and the C1D3 are administered SC (e.g., by a pump (e.g., by a patch pump)).

The doses of each dosing cycle may be administered to the subject at any suitable time interval, e.g., every week (Q1W), every two weeks (Q2W), every three weeks (Q3W), every four weeks (Q4W), every six weeks (Q6W), or every eight weeks (Q8W). For example, in some aspects, the doses of the dosing cycle are administered to the subject every four weeks (Q4W).

For example, in one aspect, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 300 mg SC, 300 mg IV, 450 mg SC, 450 mg IV, 600 mg SC, 600 mg IV, 750 mg SC, 750 mg IV, 900 mg SC, 900 mg IV, 1350 mg SC, 1350 mg IV, 1800 mg SC, 1800 mg IV, 3600 mg SC, or 3600 mg IV every four weeks (Q4W). In some instances, the dose is 300 mg SC, 600 mg SC, 900 mg IV, or 1800 mg IV.

For example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 300 mg SC (e.g., by a pump (e.g., by a patch pump)) every four weeks (Q4W).

In another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 300 mg IV every four weeks (Q4W).

In another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 450 mg SC (e.g., by a pump (e.g., by a patch pump)) every four weeks (Q4W).

In another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 450 mg IV every four weeks (Q4W).

In another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 600 mg SC (e.g., by a pump (e.g., by a patch pump)) every four weeks (Q4W).

In another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 600 mg IV every four weeks (Q4W).

In yet another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 750 mg SC (e.g., by a pump (e.g., by a patch pump)) every four weeks (Q4W).

In another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 750 mg IV every four weeks (Q4W).

In a further example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 900 mg SC (e.g., by a pump (e.g., by a patch pump)) every four weeks (Q4W).

In a further example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 900 mg IV every four weeks (Q4W).

In yet a further example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 1350 mg SC (e.g., by a pump (e.g., by a patch pump)) every four weeks (Q4W).

In yet a further example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 1350 mg IV every four weeks (Q4W).

In a still further example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 1800 mg SC (e.g., by a pump (e.g., by a patch pump)) every four weeks (Q4W).

In a still further example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 1800 mg IV every four weeks (Q4W).

In another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 3600 mg SC (e.g., by a pump (e.g., by a patch pump)) every four weeks (Q4W).

In another example, provided herein is a method of treating a patient having CSU (e.g., CSU refractory to antihistamines (e.g., sgH1-AHs)), the method including administering to a patient having CSU an anti-tryptase antibody (e.g., an anti-tryptase beta antibody) at a dose of 3600 mg IV every four weeks (Q4W).

In another aspect, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU at a dose selected from 300 mg SC, 300 mg IV, 450 mg SC, 450 mg IV, 600 mg SC, 600 mg IV, 750 mg SC, 750 mg IV, 900 mg SC, 900 mg IV, 1350 mg SC, 1350 mg IV, 1800 mg SC, 1800 mg IV, 3600 mg SC, or 3600 mg IV every four weeks (Q4W). In some instances, the dose is 300 mg SC, 600 mg SC, 900 mg IV, or 1800 mg IV.

For example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU at a dose of 300 mg SC (e.g., by a pump (e.g., by a patch pump)) every four weeks (Q4W).

In another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU at a dose of 300 mg IV every four weeks (Q4W).

In another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU at a dose of 450 mg SC (e.g., by a pump (e.g., by a patch pump)) every four weeks (Q4W).

In another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU at a dose of 450 mg IV every four weeks (Q4W).

In another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU at a dose of 600 mg SC (e.g., by a pump (e.g., by a patch pump)) every four weeks (Q4W).

In another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU at a dose of 600 mg IV every four weeks (Q4W).

In yet another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU at a dose of 750 mg SC (e.g., by a pump (e.g., by a patch pump)) every four weeks (Q4W).

In another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU at a dose of 750 mg IV every four weeks (Q4W).

In yet another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU at a dose of 900 mg SC (e.g., by a pump (e.g., by a patch pump)) every four weeks (Q4W).

In a further example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU at a dose of 900 mg IV every four weeks (Q4W).

In yet another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU at a dose of 1350 mg SC (e.g., by a pump (e.g., by a patch pump)) every four weeks (Q4W).

In yet a further example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU at a dose of 1350 mg IV every four weeks (Q4W).

In yet another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU at a dose of 1800 mg SC (e.g., by a pump (e.g., by a patch pump)) every four weeks (Q4W).

In a still further example, provided herein is an anti-tryptase antibody (e.g., an anti-tryptase beta antibody) antibody for use in treating a patient having CSU (e.g., CSU refractory to antihistamines (e.g., sgH1-AHs)), wherein the anti-tryptase antibody is for administration to a patient having CSU at a dose of 1800 mg IV every four weeks (Q4W).

In yet another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU at a dose of 3600 mg SC (e.g., by a pump (e.g., by a patch pump)) every four weeks (Q4W).

In another example, provided herein is an anti-tryptase antibody (e.g., an anti-tryptase beta antibody) for use in treating a patient having CSU (e.g., CSU refractory to antihistamines (e.g., sgH1-AHs)), wherein the anti-tryptase antibody is for administration to a patient having CSU at a dose of 3600 mg IV every four weeks (Q4W).

In another aspect, provided herein is the use of an anti-tryptase antibody (e.g., an anti-tryptase beta antibody) antibody in the manufacture of a medicament for treating a patient having CSU (e.g., CSU refractory to antihistamines (e.g., sgH1-AHs)), wherein the medicament is for administration to a patient having CSU at a dose selected from 300 mg SC, 300 mg IV, 450 mg SC, 450 mg IV, 600 mg SC, 600 mg IV, 750 mg SC, 750 mg IV, 900 mg SC, 900 mg IV, 1350 mg SC, 1350 mg IV, 1800 mg SC, 1800 mg IV, 3600 mg SC, or 3600 mg IV every four weeks (Q4W). In some instances, the dose is 300 mg SC, 600 mg SC, 900 mg IV, or 1800 mg IV.

For example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU at a dose of 300 mg SC (e.g., by a pump (e.g., by a patch pump)) every four weeks (Q4W).

For example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU at a dose of 300 mg IV every four weeks (Q4W).

In another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU at a dose of 450 mg SC (e.g., by a pump (e.g., by a patch pump)) every four weeks (Q4W).

In another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU at a dose of 450 mg IV every four weeks (Q4W).

In another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU at a dose of 600 mg SC (e.g., by a pump (e.g., by a patch pump)) every four weeks (Q4W).

In another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU at a dose of 600 mg IV every four weeks (Q4W).

In yet another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU at a dose of 750 mg SC (e.g., by a pump (e.g., by a patch pump) every four weeks (Q4W).

In a further example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU at a dose of 750 mg IV every four weeks (Q4W).

In yet another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU at a dose of 900 mg SC (e.g., by a pump (e.g., by a patch pump) every four weeks (Q4W).

In a further example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU at a dose of 900 mg IV every four weeks (Q4W).

In yet another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU at a dose of 1350 mg SC (e.g., by a pump (e.g., by a patch pump) every four weeks (Q4W).

In yet a further example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU at a dose of 1350 mg IV every four weeks (Q4W).

In yet another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU at a dose of 1800 mg SC (e.g., by a pump (e.g., by a patch pump) every four weeks (Q4W).

In a still further example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU at a dose of 1800 mg IV every four weeks (Q4W).

In yet another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU at a dose of 3600 mg SC (e.g., by a pump (e.g., by a patch pump) every four weeks (Q4W).

In another example, provided herein is the use of an anti-tryptase antibody (e.g., an anti-tryptase beta antibody) in the manufacture of a medicament for treating a patient having CSU (e.g., CSU refractory to antihistamines (e.g., sgH1-AHs)), wherein the medicament is for administration to a patient having CSU at a dose of 3600 mg IV every four weeks (Q4W).

Each dosing cycle may have any suitable length.

For example, in some aspects, each dosing cycle may have a length of about 57 days.

The doses of each dosing cycle may be administered on any suitable day(s) of the dosing cycle. For example, in some aspects, the C1D1 is administered on Day 1 of the dosing cycle, the C1D2 is administered on Day 29 (±1 day) of the dosing cycle, and the C1D3 is administered on Day 57 (±1 day) of the dosing cycle.

In other aspects, the dosing cycle may have a length of about 48 weeks. For example, in some aspects, the doses of the dosing cycle are administered every four weeks (Q4W) for 48 weeks. For example, in some aspects, the C1D1 is administered on Week 0 of the dosing cycle, a C1D2 is administered on Week 4 of the dosing cycle, a C1D3 is administered on Week 8 of the dosing cycle, a C1D3 is administered on Week 12 of the dosing cycle, a C1D4 is administered on Week 16 of the dosing cycle, a C1D5 is administered on Week 20 of the dosing cycle, a C1D6 is administered on Week 24 of the dosing cycle, a C1D7 is administered on Week 28 of the dosing cycle, a C1D8 is administered on Week 32 of the dosing cycle, a C1D9 is administered on Week 36 of the dosing cycle, a C1D10 is administered on Week 40 of the dosing cycle, a C1D11 is administered on Week 44 of the dosing cycle, and a C1D12 is administered on Week 48 of the dosing cycle.

In other aspects, the dosing cycle may continue indefinitely, e.g., while the patient is responding to the treatment or until the patient experiences a relapse. For example, the patient may be administered an anti-tryptase antibody (e.g., anti-tryptase beta antibody), e.g., every week (Q1W), every two weeks (Q2W), every three weeks (Q3W), every four weeks (Q4W), every six weeks (Q6W), or every eight weeks (Q8W), indefinitely. For example, in some aspects, the doses of the dosing cycle are administered to the subject every four weeks (Q4W) indefinitely.

The dosing regimens described herein may include any suitable number of dosing cycles. For example, in some aspects, the dosing regimen includes or consists of one dosing cycle. In other aspects, the dosing regimen may include more than one dosing cycle (e.g., 2, 3, 4, 5, 6, 7, 8, 9, 10, 11, 12, 13, 14, 15, 16, 17, 18, 19, 20, or more dosing cycles).

Any suitable anti-tryptase antibody (e.g., anti-tryptase beta antibody) may be used in any of the aspects described herein. For example, any of the anti-tryptase antibodies described in Section IV, Subsection A below can be used. In some aspects, the anti-tryptase antibody may be any anti-tryptase antibody described in U.S. Pat. Nos. 10,738,131 or 10,752,703; in U.S. Patent Application Publication No. US 2018/0230233; or in International Patent Application Publication No. WO 2018/148585.

For example, any of the anti-tryptase (e.g., anti-tryptase beta) antibodies may include one, two, three, four, five, or all six of the following complementarity determining regions (CDRs): (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In one aspect, provided herein is a method of treating a patient having CSU (e.g., CSU refractory to antihistamines (e.g., sgH1-AHs)), the method comprising administering to a patient having CSU an anti-tryptase beta antibody in a dosing regimen comprising a dosing cycle, wherein the dosing cycle comprises a first dose (C1D1) of the anti-tryptase beta antibody selected from 300 mg SC, 600 mg SC, 900 mg IV, or 1800 mg IV, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

For example, provided herein is a method of treating a patient having CSU (e.g., CSU refractory to antihistamines (e.g., sgH1-AHs)), the method comprising administering to a patient having CSU an anti-tryptase beta antibody in a dosing regimen comprising a dosing cycle, wherein the dosing cycle comprises a first dose (C1D1) of the anti-tryptase beta antibody of 300 mg SC, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is a method of treating a patient having CSU (e.g., CSU refractory to antihistamines (e.g., sgH1-AHs)), the method comprising administering to a patient having CSU an anti-tryptase beta antibody in a dosing regimen comprising a dosing cycle, wherein the dosing cycle comprises a first dose (C1D1) of the anti-tryptase beta antibody of 600 mg SC, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is a method of treating a patient having CSU (e.g., CSU refractory to antihistamines (e.g., sgH1-AHs)), the method comprising administering to a patient having CSU an anti-tryptase beta antibody in a dosing regimen comprising a dosing cycle, wherein the dosing cycle comprises a first dose (C1D1) of the anti-tryptase beta antibody of 900 mg IV, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is a method of treating a patient having CSU (e.g., CSU refractory to antihistamines (e.g., sgH1-AHs)), the method comprising administering to a patient having CSU an anti-tryptase beta antibody in a dosing regimen comprising a dosing cycle, wherein the dosing cycle comprises a first dose (C1D1) of the anti-tryptase beta antibody of 1800 mg IV, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, provided herein is an anti-tryptase beta antibody for use in treating a patient having CSU (e.g., CSU refractory to antihistamines (e.g., sgH1-AHs)), wherein the anti-tryptase beta antibody is for administration to a patient having CSU in a dosing regimen comprising a dosing cycle, wherein the dosing cycle comprises a first dose (C1D1) of the anti-tryptase beta antibody selected from 300 mg SC, 600 mg SC, 900 mg IV, or 1800 mg IV, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

For example, provided herein is an anti-tryptase beta antibody for use in treating a patient having CSU (e.g., CSU refractory to antihistamines (e.g., sgH1-AHs)), wherein the anti-tryptase beta antibody is for administration to a patient having CSU in a dosing regimen comprising a dosing cycle, wherein the dosing cycle comprises a first dose (C1D1) of the anti-tryptase beta antibody of 300 mg SC, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is an anti-tryptase beta antibody for use in treating a patient having CSU (e.g., CSU refractory to antihistamines (e.g., sgH1-AHs)), wherein the anti-tryptase beta antibody is for administration to a patient having CSU in a dosing regimen comprising a dosing cycle, wherein the dosing cycle comprises a first dose (C1D1) of the anti-tryptase beta antibody of 600 mg SC, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is an anti-tryptase beta antibody for use in treating a patient having CSU (e.g., CSU refractory to antihistamines (e.g., sgH1-AHs)), wherein the anti-tryptase beta antibody is for administration to a patient having CSU in a dosing regimen comprising a dosing cycle, wherein the dosing cycle comprises a first dose (C1D1) of the anti-tryptase beta antibody of 900 mg IV, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is an anti-tryptase beta antibody for use in treating a patient having CSU (e.g., CSU refractory to antihistamines (e.g., sgH1-AHs)), wherein the anti-tryptase beta antibody is for administration to a patient having CSU in a dosing regimen comprising a dosing cycle, wherein the dosing cycle comprises a first dose (C1D1) of the anti-tryptase beta antibody of 1800 mg IV, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, provided herein is the use of an anti-tryptase beta antibody in the manufacture of a medicament for treating a patient having CSU (e.g., CSU refractory to antihistamines (e.g., sgH1-AHs)), wherein the medicament is for administration to a patient having CSU in a dosing regimen comprising a dosing cycle, wherein the dosing cycle comprises a first dose (C1D1) of the anti-tryptase beta antibody selected from 300 mg SC, 600 mg SC, 900 mg IV, or 1800 mg IV, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

For example, provided herein is the use of an anti-tryptase beta antibody in the manufacture of a medicament for treating a patient having CSU (e.g., CSU refractory to antihistamines (e.g., sgH1-AHs)), wherein the medicament is for administration to a patient having CSU in a dosing regimen comprising a dosing cycle, wherein the dosing cycle comprises a first dose (C1D1) of the anti-tryptase beta antibody of 300 mg SC, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is the use of an anti-tryptase beta antibody in the manufacture of a medicament for treating a patient having CSU (e.g., CSU refractory to antihistamines (e.g., sgH1-AHs)), wherein the medicament is for administration to a patient having CSU in a dosing regimen comprising a dosing cycle, wherein the dosing cycle comprises a first dose (C1D1) of the anti-tryptase beta antibody of 600 mg SC, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is the use of an anti-tryptase beta antibody in the manufacture of a medicament for treating a patient having CSU (e.g., CSU refractory to antihistamines (e.g., sgH1-AHs)), wherein the medicament is for administration to a patient having CSU in a dosing regimen comprising a dosing cycle, wherein the dosing cycle comprises a first dose (C1D1) of the anti-tryptase beta antibody of 900 mg IV, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is the use of an anti-tryptase beta antibody in the manufacture of a medicament for treating a patient having CSU (e.g., CSU refractory to antihistamines (e.g., sgH1-AHs)), wherein the medicament is for administration to a patient having CSU in a dosing regimen comprising a dosing cycle, wherein the dosing cycle comprises a first dose (C1D1) of the anti-tryptase beta antibody of 1800 mg IV, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In some aspects, the dosing cycle further comprises a second dose (C1D2) and a third dose (C1D3) of the anti-tryptase beta antibody, wherein the C1D2 and the C1D3 are each equal to the C1D1.

In some aspects, the doses of the dosing cycle are administered to the subject every four weeks (Q4W).

In some aspects, the dosing cycle has a length of about 57 days.

In some aspects, the C1D1 is administered on Day 1 of the dosing cycle, the C1D2 is administered on Day 29 (±1 day) of the dosing cycle, and the C1D3 is administered on Day 57 (±1 day) of the dosing cycle.

In some aspects, the dosing regimen consists of one dosing cycle.

In another aspect, provided herein is a method of treating a patient having CSU (e.g., CSU refractory to antihistamines (e.g., sgH1-AHs)), the method comprising administering to a patient having CSU an anti-tryptase beta antibody in a dosing regimen comprising a dosing cycle, wherein the dosing cycle comprises between two and six doses, wherein a total of about 300 mg to about 21,600 mg of the anti-tryptase antibody is administered SC or IV to the patient in the dosing cycle, and wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, provided herein is an anti-tryptase beta antibody for use in treating a patient having CSU (e.g., CSU refractory to antihistamines (e.g., sgH1-AHs)), wherein the anti-tryptase beta antibody is for administration to a patient having CSU in a dosing regimen comprising a dosing cycle, wherein the dosing cycle comprises between two and six doses, wherein a total of about 300 mg to about 21,600 mg of the anti-tryptase antibody is administered SC or IV to the patient in the dosing cycle, and wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, provided herein is the use of an anti-tryptase beta antibody in the manufacture of a medicament for treating a patient having CSU (e.g., CSU refractory to antihistamines (e.g., sgH1-AHs)), wherein the medicament is for administration to a patient having CSU in a dosing regimen comprising a dosing cycle, wherein the dosing cycle comprises between two and six doses, wherein a total of about 300 mg to about 21,600 mg of the anti-tryptase antibody is administered SC or IV to the patient in the dosing cycle, wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, provided herein is a method of treating a patient having CSU (e.g., CSU refractory to antihistamines (e.g., sgH1-AHs)), the method comprising administering to a patient having CSU an anti-tryptase beta antibody in a dosing regimen comprising a dosing cycle, wherein the dosing cycle comprises administering the anti-tryptase beta antibody at a dose selected from 300 mg SC, 600 mg SC, 900 mg IV, or 1800 mg IV every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

For example, provided herein is a method of treating a patient having CSU (e.g., CSU refractory to antihistamines (e.g., sgH1-AHs)), the method comprising administering to a patient having CSU an anti-tryptase beta antibody in a dosing regimen comprising a dosing cycle, wherein the dosing cycle comprises administering the anti-tryptase beta antibody at a dose of 300 mg SC every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is a method of treating a patient having CSU (e.g., CSU refractory to antihistamines (e.g., sgH1-AHs)), the method comprising administering to a patient having CSU an anti-tryptase beta antibody in a dosing regimen comprising a dosing cycle, wherein the dosing cycle comprises administering the anti-tryptase beta antibody at a dose of 600 mg SC every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is a method of treating a patient having CSU (e.g., CSU refractory to antihistamines (e.g., sgH1-AHs)), the method comprising administering to a patient having CSU an anti-tryptase beta antibody in a dosing regimen comprising a dosing cycle, wherein the dosing cycle comprises administering the anti-tryptase beta antibody at a dose of 900 mg IV every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is a method of treating a patient having CSU (e.g., CSU refractory to antihistamines (e.g., sgH1-AHs)), the method comprising administering to a patient having CSU an anti-tryptase beta antibody in a dosing regimen comprising a dosing cycle, wherein the dosing cycle comprises administering the anti-tryptase beta antibody at a dose of 1800 mg IV every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, provided herein is an anti-tryptase beta antibody for use in treating a patient having CSU (e.g., CSU refractory to antihistamines (e.g., sgH1-AHs)), wherein the anti-tryptase beta antibody is for administration to a patient having CSU in a dosing regimen comprising a dosing cycle, wherein the dosing cycle comprises administering the anti-tryptase beta antibody at a dose selected from 300 mg SC, 600 mg SC, 900 mg IV, or 1800 mg IV every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

For example, provided herein is an anti-tryptase beta antibody for use in treating a patient having CSU (e.g., CSU refractory to antihistamines (e.g., sgH1-AHs)), wherein the anti-tryptase beta antibody is for administration to a patient having CSU in a dosing regimen comprising a dosing cycle, wherein the dosing cycle comprises administering the anti-tryptase beta antibody at a dose of 300 mg SC every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is an anti-tryptase beta antibody for use in treating a patient having CSU (e.g., CSU refractory to antihistamines (e.g., sgH1-AHs)), wherein the anti-tryptase beta antibody is for administration to a patient having CSU in a dosing regimen comprising a dosing cycle, wherein the dosing cycle comprises administering the anti-tryptase beta antibody at a dose of 600 mg SC every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is an anti-tryptase beta antibody for use in treating a patient having CSU (e.g., CSU refractory to antihistamines (e.g., sgH1-AHs)), wherein the anti-tryptase beta antibody is for administration to a patient having CSU in a dosing regimen comprising a dosing cycle, wherein the dosing cycle comprises administering the anti-tryptase beta antibody at a dose of 900 mg IV every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is an anti-tryptase beta antibody for use in treating a patient having CSU (e.g., CSU refractory to antihistamines (e.g., sgH1-AHs)), wherein the anti-tryptase beta antibody is for administration to a patient having CSU in a dosing regimen comprising a dosing cycle, wherein the dosing cycle comprises administering the anti-tryptase beta antibody at a dose of 1800 mg IV every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another aspect, provided herein is the use of an anti-tryptase beta antibody in the manufacture of a medicament for treating a patient having CSU (e.g., CSU refractory to antihistamines (e.g., sgH1-AHs)), wherein the medicament is for administration to a patient having CSU in a dosing regimen comprising a dosing cycle, wherein the dosing cycle comprises administering the anti-tryptase beta antibody at a dose selected from 300 mg SC, 600 mg SC, 900 mg IV, or 1800 mg IV every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

For example, provided herein is the use of an anti-tryptase beta antibody in the manufacture of a medicament for treating a patient having CSU (e.g., CSU refractory to antihistamines (e.g., sgH1-AHs)), wherein the medicament is for administration to a patient having CSU in a dosing regimen comprising a dosing cycle, wherein the dosing cycle comprises administering the anti-tryptase beta antibody at a dose of 300 mg SC every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is the use of an anti-tryptase beta antibody in the manufacture of a medicament for treating a patient having CSU (e.g., CSU refractory to antihistamines (e.g., sgH1-AHs)), wherein the medicament is for administration to a patient having CSU in a dosing regimen comprising a dosing cycle, wherein the dosing cycle comprises administering the anti-tryptase beta antibody at a dose of 600 mg SC every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is the use of an anti-tryptase beta antibody in the manufacture of a medicament for treating a patient having CSU (e.g., CSU refractory to antihistamines (e.g., sgH1-AHs)), wherein the medicament is for administration to a patient having CSU in a dosing regimen comprising a dosing cycle, wherein the dosing cycle comprises administering the anti-tryptase beta antibody at a dose of 900 mg IV every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In another example, provided herein is the use of an anti-tryptase beta antibody in the manufacture of a medicament for treating a patient having CSU (e.g., CSU refractory to antihistamines (e.g., sgH1-AHs)), wherein the medicament is for administration to a patient having CSU in a dosing regimen comprising a dosing cycle, wherein the dosing cycle comprises administering the anti-tryptase beta antibody at a dose of 1800 mg IV every four weeks (Q4W), wherein the anti-tryptase beta antibody comprises the following six CDRs: (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In any of the aspects provided herein, the antibody may include (a) a heavy chain variable (VH) domain including an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 7; (b) a light chain variable (VL) domain including an amino acid sequence having at least 90%, at least 91%, at least 92%, at least 93%, at least 94%, at least 95%, at least 96%, at least 97%, at least 98%, or at least 99% identity to the amino acid sequence of SEQ ID NO: 8; or (c) a VH domain as in (a) and a VL domain as in (b).

For example, in some aspects, the antibody may include (a) a heavy chain variable (VH) domain including an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 7. In some aspects, the VH domain includes the amino acid sequence of SEQ ID NO: 7.

In another example, in some aspects, the antibody may include (b) a light chain variable (VL) domain including an amino acid sequence having at least 90%, at least 91%, at least 92%, at least 93%, at least 94%, at least 95%, at least 96%, at least 97%, at least 98%, or at least 99% identity to the amino acid sequence of SEQ ID NO: 8. In some aspects, the VL domain includes the amino acid sequence of SEQ ID NO: 8.

In any of the aspects described herein, the VH domain may include the amino acid sequence of SEQ ID NO: 7 and the VL domain includes the amino acid sequence of SEQ ID NO: 8.

In another example, in any of the aspects described herein, the antibody may include (a) a heavy chain having at least 90%, at least 91%, at least 92%, at least 93%, at least 94%, at least 95%, at least 96%, at least 97%, at least 98%, or at least 99% identity to the amino acid sequence of SEQ ID NO: 9 and (b) a light chain having at least 90%, at least 91%, at least 92%, at least 93%, at least 94%, at least 95%, at least 96%, at least 97%, at least 98%, or at least 99% identity to the amino acid sequence of SEQ ID NO: 10. For example, in some aspects, the antibody may include (a) a heavy chain including the amino acid sequence of SEQ ID NO: 9 and (b) a light chain including the amino acid sequence of SEQ ID NO: 10.

In another example, in any of the aspects described herein, the antibody may include (a) a heavy chain having at least 90%, at least 91%, at least 92%, at least 93%, at least 94%, at least 95%, at least 96%, at least 97%, at least 98%, or at least 99% identity to the amino acid sequence of SEQ ID NO: 11 and (b) a light chain having at least 90%, at least 91%, at least 92%, at least 93%, at least 94%, at least 95%, at least 96%, at least 97%, at least 98%, or at least 99% identity to the amino acid sequence of SEQ ID NO: 10. For example, in some aspects, the antibody may include (a) a heavy chain including the amino acid sequence of SEQ ID NO: 11 and (b) a light chain including the amino acid sequence of SEQ ID NO: 10.

The methods, compositions for use (e.g., anti-tryptase antibodies for use), and uses of the present disclosure may be used for treating any suitable type of CSU.

In some aspects, the CSU is refractory to antihistamines.

In some aspects, the CSU is refractory to second-generation H1 antihistamines (sgH1-AHs).

In some aspects, the patient: (i) has had a CSU diagnosis for greater than or equal to (≥) 6 months; (ii) has presence of itch and hives for greater than (>) 6 consecutive weeks at any time prior to treatment despite current use of sgH1-AHs, consistent with standard of care during this time period; (iii) has received stable doses of sgH1-AHs, consistent with standard of care therapy for CSU, starting at least 14 (−4/+2 days) consecutive days prior to treatment; and/or (iv) has a Urticaria Activity Score summed over 7 days (UAS7) symptom score of ≥16 during the 7 days prior to the C1D1.

In some aspects, the patient has a UAS7 symptom score of ≥16.

In some aspects, the patient is Chronic Urticaria Index (CU Index®)-positive.

In some aspects, the patient is receiving background sgH1-AH therapy. For example, in some aspects, the patient may be receiving cetirizine, levocetirizine, fexofenadine, loratadine, desloratadine, rupatadine, or bilastine.

In some aspects, the background sgH1-AH therapy comprises cetirizine 10-40 mg once a day (QD), levocetirizine 5-20 mg QD, fexofenadine 180-720 mg QD, loratadine 10-40 mg QD, desloratadine 5-20 mg QD, rupatadine 10-40 mg QD, or bilastine 20-80 mg QD.

In some aspects, the patient receives a single dose of rescue therapy within a 24-hour period if symptoms worsen.

In some aspects, the rescue therapy comprises up to 10 mg loratadine or up to 10 mg cetirizine.

In some aspects, the treating results in an improvement from baseline in the patient's UAS7 at Week 12 compared to placebo.

In some aspects, (i) the treatment results in well-controlled urticaria (UAS7 less than or equal to (≤) 6 at Week 12); or (ii) the treatment results in a complete response (UAS7=0) at Week 12.

Any of the aspects disclosed herein may include administering one or more additional therapeutic agents to the patient. Any suitable additional therapeutic agent(s) may be used, e.g., antihistamines (e.g., sgH1-AHs, e.g., cetirizine, levocetirizine, fexofenadine, loratadine, desloratadine, rupatadine, bilastine, or a combination thereof), an anti-IgE antibody (e.g., omalizumab (XOLAIR®) or ligelizumab (also known as QGE031)), a Spleen tyrosine kinase (Syk) inhibitor (e.g., GSK2646264), an anti-sialic acid-binding immunoglobulin-like lectin 8 (Siglec-8) antibody (e.g., lirentelimab (also known as AK002)), a Bruton's tyrosine kinase (BTK) inhibitor (e.g., ibrutinib or fenebrutinib), a leukotriene receptor antagonist (LTRA) (e.g., montelukast, zafirlukast, or pranlukast), a leukotriene synthesis inhibitor (e.g., zileuton), a chemoattractant receptor-homologous molecule expressed on T helper type 2 (CRTh2) antagonist (e.g., AZD1981), an interleukin 1 (IL-1) antagonist (e.g., an anti-IL-1 antibody, e.g., an anti-IL-1ß antibody, e.g., canakinumab), an interleukin 5 (IL-5) antagonist (e.g., an anti-IL-5 antibody (e.g., mepolizumab) or an anti-IL-5 receptor alpha antibody (e.g., benralizumab)), dapsone, cyclosporin A, or any combination thereof. Exemplary additional therapeutic agents for treatment of CSU are described, e.g., in Min et al. Allergy Asthma Immunol. Res. 11(4):470-481, 2019. In some examples, an antihistamine (e.g., sgH1-AHs, e.g., cetirizine, levocetirizine, fexofenadine, loratadine, desloratadine, rupatadine, bilastine, or a combination thereof) may be administered at a higher dose than a standard dose for the antihistamine, e.g., up to a 4-fold higher dose than a standard dose. The one or more additional therapeutic agents may be standard of care for CSU (e.g., antihistamines (e.g., sgH1-AHs, e.g., cetirizine, levocetirizine, fexofenadine, loratadine, desloratadine, rupatadine, bilastine, or a combination thereof)). A person of skill in the art will be able to select a suitable standard of care as appropriate.

The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. The combined administration includes co-administration, using separate formulations or a single pharmaceutical formulation, and consecutive administration in either order, wherein preferably there is a time period while both (or all) active agents simultaneously exert their biological activities. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together. When administered sequentially, the combination may be administered in two or more administrations.

Such combination therapies noted above encompass combined administration (where two or more therapeutic agents are included in the same or separate formulations), and separate administration, in which case, administration of an agent (e.g., an anti-tryptase antibody), or a pharmaceutical composition thereof, can occur prior to, simultaneously, and/or following, administration of the additional therapeutic agent(s). In one aspect, administration of an agent (e.g., an anti-tryptase antibody), or a pharmaceutical composition thereof, and administration of an additional therapeutic agent occur within about one month; or within about one, two, or three weeks; or within about one, two, three, four, five, or six days; or within about 1, 2, 3, 4, 5, 6, 7, 8, or 9 hours; or within about 1, 5, 10, 20, 30, 40, or 50 minutes, of each other. For aspects involving sequential administration, the agent (e.g., an anti-tryptase antibody) may be administered prior to or after administration of the additional therapeutic agent(s).

In any of the aspects described herein, the anti-tryptase antibody, and any additional therapeutic agent, can be administered by any suitable means, including parenterally, intraperitoneally, intramuscularly, intravenously, intradermally, percutaneously, intraarterially, intralesionally, intracranially, intraarticularly, intraprostatically, intrapleurally, intratracheally, intrathecally, intranasally, intravaginally, intrarectally, topically, intratumorally, peritoneally, subcutaneously, subconjunctivally, intravesicularly, mucosally, intrapericardially, intraumbilically, intraocularly, intraorbitally, orally, topically, transdermally, intravitreally, periocularly, conjunctivally, subtenonly, intracamerally, subretinally, retrobulbarly, intracanalicularly, by inhalation, by injection, by implantation, by infusion, by continuous infusion, by localized perfusion bathing target cells directly, by catheter, by lavage, in cremes, or in lipid compositions. The administration may be systemic or local. In addition, the antagonist may suitably be administered by pulse infusion, e.g., with declining doses of the antagonist.

In any of the aspects described herein, the anti-tryptase antibody, and any additional therapeutic agent, can be administered intravenously.

In any of the aspects described herein, the anti-tryptase antibody, and any additional therapeutic agent, can be administered subcutaneously (e.g., by a pump (e.g., by a patch pump)).

Any therapeutic agent, e.g., an anti-tryptase antibody, any additional therapeutic agent, or pharmaceutical compositions thereof, would be formulated, dosed, and administered in a fashion consistent with good medical practice. Dosages for anti-tryptase antibodies are disclosed herein. Dosages for additional therapeutic agents are known in the art. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The therapeutic agent (e.g., an anti-tryptase antibody), or pharmaceutical composition thereof, need not be, but is optionally formulated with one or more agents currently used to prevent or treat the disorder in question (e.g., CSU (e.g., CSU refractory to antihistamines (e.g., sgH1-AHs))). The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

As one example, for the prevention or treatment of disease, the appropriate dosage of an antibody, when used alone or in combination with one or more other additional therapeutic agents, will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g., 0.1 mg/kg to 10 mg/kg) of antibody can be an initial candidate dosage for administration to the patient, whether, for example, by one or more separate administrations, or by continuous infusion. One typical daily dosage might range from about 1 μg/kg to 200 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. For example, a dose may be administered once per month. An initial higher loading dose, followed by one or more lower doses, may be administered. However, other dosage regimens may be useful. The progress of this therapy is easily monitored by conventional techniques and assays. In some aspects, a dose of about 50 mg/mL to about 200 mg/ml (e.g., about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, or about 200 mg/ml of an antibody may be administered.

### IV. Compositions and Pharmaceutical Formulations

Any suitable composition (e.g., anti-tryptase antibody) or a pharmaceutical formulation thereof can be used in the methods, compositions for use, and uses described herein. Non-limiting examples suitable for the methods, compositions for use, and uses described herein are described further below.

### A. Anti-Tryptase Antibodies

Any suitable anti-tryptase antibody can be used in the methods, compositions for use, and uses of the invention. For example, the anti-tryptase antibody may be any anti-tryptase antibody described in U.S. Pat. Nos. 10,738,131 and 10,752,703; in U.S. Patent Application Publication No. US 2018/0230233; or in International Patent Application Publication No. WO 2018/148585.

In some aspects, the anti-tryptase antibody (e.g., the anti-tryptase beta antibody) can include at least one, at least two, at least three, at least four, at least five, or all six CDRs selected from (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6), or a combination of one or more of the above CDRs and one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) to any one of SEQ ID NOs: 1-6. For example, in some aspects, the anti-tryptase antibody includes (a) an CDR-H1 comprising the amino acid sequence of DYGMV (SEQ ID NO: 1); (b) an CDR-H2 comprising the amino acid sequence of FISSGSSTVYYADTMKG (SEQ ID NO: 2); (c) an CDR-H3 comprising the amino acid sequence of RNYDDWYFDV (SEQ ID NO: 3); (d) an CDR-L1 comprising the amino acid sequence of SASSSVTYMY (SEQ ID NO: 4); (e) an CDR-L2 comprising the amino acid sequence of RTSDLAS (SEQ ID NO: 5); and (f) an CDR-L3 comprising the amino acid sequence of QHYHSYPLT (SEQ ID NO: 6).

In some aspects, the anti-tryptase antibody (e.g., the anti-tryptase beta antibody) can include (a) a heavy chain variable (VH) domain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 7; (b) a light chain variable (VL) domain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 8; or (c) a VH domain as in (a) and a VL domain as in (b). For example, in some aspects, the VH domain comprises the amino acid sequence of SEQ ID NO: 7. In some aspects, the VL domain comprises the amino acid sequence of SEQ ID NO: 8. In particular aspects, the VH domain comprises the amino acid sequence of SEQ ID NO: 7 and the VL domain comprises the amino acid sequence of SEQ ID NO: 8.

In some aspects, the anti-tryptase antibody (e.g., the anti-tryptase beta antibody) can include (a) a heavy chain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 9 and (b) a light chain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 10. For example, in some aspects, the anti-tryptase antibody (e.g., the anti-tryptase beta antibody) includes (a) a heavy chain comprising the amino acid sequence of SEQ ID NO: 9 and (b) a light chain comprising the amino acid sequence of SEQ ID NO: 10.

In other aspects, the anti-tryptase antibody (e.g., the anti-tryptase beta antibody) can include (a) a heavy chain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 11 and (b) a light chain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 10. For example, in some aspects, the anti-tryptase antibody (e.g., the anti-tryptase beta antibody) includes (a) a heavy chain comprising the amino acid sequence of SEQ ID NO: 11 and (b) a light chain comprising the amino acid sequence of SEQ ID NO: 10.

In some aspects, the anti-tryptase antibody is an antibody that binds to the same epitope as any one of the preceding antibodies. In some aspects, whether the antibody binds to the same epitope or competes for binding to human tryptase beta 1 is determined by an epitope binning assay. In some aspects, the epitope binning assay is an OCTET® epitope binning assay such as described in Example 3, Section C of WO 2018/148585. In some aspects, human tryptase beta 1 monomer protein is biotinylated at Lys residue by reacting with NHS-PEG4-biotin. Biotinylated monomer is diluted to 5 g/ml in kinetics buffer (ForteBio, Inc.) and immobilized onto streptavidin sensor tips (ForteBio, Inc.). After the immobilization step, human tryptase beta 1-immobilized sensors are saturated with the first antibody, diluted at 10-20 μg/ml, followed by binding with second antibody diluted at 2.5 μg/ml. In some aspects, the epitope binning assay is performed at 30° C.

In some aspects, the anti-tryptase antibody is an antibody that competes for binding with, or cross-blocks or is cross-blocked by any one of the preceding antibodies.

It is expressly contemplated that any such anti-tryptase antibodies for use in any of the aspects enumerated herein may have any of the features, singly or in combination, described in Sections 1-7 below.

### 1. Antibody Affinity

In certain aspects, an antibody provided herein (e.g., an anti-tryptase antibody) has a dissociation constant (K_{D}) of ≤1 μM, ≤100 nM, ≤10 nM, ≤1 nM, ≤0.1 nM, ≤0.01 nM, ≤1 pM, or ≤0.1 pM (e.g., 10⁻⁶ M or less, e.g., from 10⁻⁶ M to 10⁻⁹ M or less, e.g., from 10⁻⁹ M to 10⁻¹³ M or less). For example, in some aspects, an anti-tryptase antibody binds to tryptase (e.g., human tryptase, e.g., human tryptase beta) with a K_{D} of about 100 nM or lower (e.g., 100 nM or lower, 10 nM or lower, 1 nM or lower, 100 pM or lower, 10 pM or lower, 1 pM or lower, or 0.1 pM or lower). In some aspects, the antibody binds tryptase (e.g., human tryptase, e.g., human tryptase beta) with a K_{D} of 10 nM or lower (e.g., 10 nM or lower, 1 nm or lower, 100 pM or lower, 10 pM or lower, 1 pM or lower, or 0.1 pM or lower). In some aspects, the antibody binds tryptase (e.g., human tryptase, e.g., human tryptase beta) with a K_{D} of 1 nM or lower (e.g., 1 nm or lower, 100 pM or lower, 10 pM or lower, 1 pM or lower, or 0.1 pM or lower). In some aspects, any of the anti-tryptase antibodies described above or herein binds to tryptase (e.g., human tryptase, e.g., human tryptase beta) with a K_{D} of about 0.5 nM or lower (e.g., 0.5 nm or lower, 400 pM or lower, 300 pM or lower, 200 pM or lower, 100 pM or lower, 50 pM or lower, 25 pM or lower, 10 pM or lower, 1 pM or lower, or 0.1 pM or lower). In some aspects, the antibody binds tryptase (e.g., human tryptase, e.g., human tryptase beta) with a K_{D} between about 0.1 nM to about 0.5 nM (e.g., about 0.1 nM, about 0.2 nM, about 0.3 nM, about 0.4 nM, or about 0.5 nM). In some aspects, the antibody binds tryptase (e.g., human tryptase, e.g., human tryptase beta) with a K_{D} of about 0.4 nM. In some aspects, the antibody binds tryptase (e.g., human tryptase, e.g., human tryptase beta) with a K_{D} of about 0.18 nM. Any of the other antibodies described herein may bind to its antigen with affinities as described above with respect to anti-tryptase antibodies.

In one aspect, K_{D} is measured by a radiolabeled antigen binding assay (RIA). In one aspect, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al. J. Mol. Biol. 293:865-881, 1999). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al. Cancer Res. 57:4593-4599, 1997). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN®-20) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another aspect, K_{D} is measured using a BIACORE® surface plasmon resonance assay. For example, an assay using a BIACORE®-2000 or a BIACORE @-3000 (BIAcore, Inc., Piscataway, NJ) is performed at 25° ° C. with immobilized antigen CM5 chips at ˜10 response units (RU). In one aspect, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in phosphate buffered saline (PBS) with 0.05% polysorbate 20 (TWEEN®-20) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, for example, Chen et al. (J. Mol. Biol. 293:865-881, 1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

In some aspects, K_{D} is measured using a BIACORE® SPR assay. In some aspects, the SPR assay can use a BIAcore® T200 or an equivalent device. In some aspects, BIAcore® Series S CM5 sensor chips (or equivalent sensor chips) are immobilized with monoclonal mouse anti-human IgG (Fc) antibody and anti-tryptase antibodies are subsequently captured on the flow cell. Serial 3-fold dilutions of the His-tagged human tryptase beta 1 monomer (SEQ ID NO: 128) are injected at a flow rate of 30 μl/min. Each sample is analyzed with 3 min association and 10 min dissociation. The assay is performed at 25° C. After each injection, the chip is regenerated using 3 M MgCl₂. Binding response is corrected by subtracting the response units (RU) from a flow cell capturing an irrelevant IgG at similar density. A 1:1 Langmuir model of simultaneous fitting of kₒₙ and k_{off} is used for kinetics analysis.

### 2. Antibody Fragments

In certain aspects, an antibody provided herein (e.g., an anti-tryptase antibody) is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134, 2003; and Hollinger et al. Proc. Natl. Acad. Sci. USA 90: 6444-6448, 1993. Triabodies and tetrabodies are also described in Hudson et al. Nat. Med. 9:129-134, 2003.

Single-domain antibodies are antibody fragments comprising all or a portion of the heavy chain variable domain or all or a portion of the light chain variable domain of an antibody. In certain aspects, a single-domain antibody is a human single-domain antibody (see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

### 3. Chimeric and Humanized Antibodies

In certain aspects, an antibody provided herein (e.g., an anti-tryptase antibody) is a chimeric antibody. Certain chimeric antibodies are described, e.g., in U.S. Pat. No. 4,816,567; and Morrison et al. Proc. Natl. Acad. Sci. USA, 81:6851-6855, 1984). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the HVR residues are derived), for example, to restore or improve antibody specificity or affinity.

Humanized antibodies and methods of making them are reviewed, for example, in Almagro et al. Front. Biosci. 13:1619-1633, 2008, and are further described, e.g., in Riechmann et al. Nature 332:323-329, 1988; Queen et al. Proc. Natl. Acad. Sci. USA 86:10029-10033, 1989; U.S. Pat. Nos. 5,821,337, 7,527,791, 6,982,321, and 7,087,409; Kashmiri et al. Methods 36:25-34, 2005 (describing specificity determining region (SDR) grafting); Padlan, Mol. Immunol. 28:489-498, 1991 (describing “resurfacing”); Dall'Acqua et al. Methods 36:43-60, 2005 (describing “FR shuffling”); and Osbourn et al. Methods 36:61-68, 2005 and Klimka et al. Br. J. Cancer, 83:252-260, 2000 (describing the “guided selection” approach to FR shuffling).

Human framework regions that may be used for humanization include but are not limited to: framework regions selected using the “best-fit” method (see, e.g., Sims et al. J. Immunol. 151:2296, 1993); framework regions derived from the consensus sequence of human antibodies of a particular subgroup of light or heavy chain variable regions (see, e.g., Carter et al. Proc. Natl. Acad. Sci. USA, 89:4285, 1992; and Presta et al. J. Immunol., 151:2623, 1993); human mature (somatically mutated) framework regions or human germline framework regions (see, e.g., Almagro et al. Front. Biosci. 13:1619-1633, 2008); and framework regions derived from screening FR libraries (see, e.g., Baca et al. J. Biol. Chem. 272:10678-10684, 1997 and Rosok et al. J. Biol. Chem. 271:22611-22618, 1996).

### 4. Human Antibodies

In certain aspects, an antibody provided herein (e.g., an anti-tryptase antibody) is a human antibody. Human antibodies can be produced using various techniques known in the art. Human antibodies are described generally in van Dijk et al. Curr. Opin. Pharmacol. 5:368-74, 2001 and Lonberg, Curr. Opin. Immunol. 20:450-459, 2008.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125, 2005. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001, 1984; Brodeur et al. Monoclonal Antibody Production Techniques and Applications, pp. 51-63 (Marcel Dekker, Inc., New York, 1987); and Boerner et al. J. Immunol. 147: 86, 1991). Human antibodies generated via human B-cell hybridoma technology are also described in Li et al. Proc. Natl. Acad. Sci. USA, 103:3557-3562, 2006. Additional methods include those described, for example, in U.S. Pat. No. 7,189,826 (describing production of monoclonal human IgM antibodies from hybridoma cell lines) and Ni, Xiandai Mianyixue, 26(4): 265-268, 2006 (describing human-human hybridomas). Human hybridoma technology (Trioma technology) is also described in Vollmers et al. Histology and Histopathology 20(3):927-937, 2005 and Vollmers et al. Methods and Findings in Experimental and Clinical Pharmacology 27(3):185-91, 2005.

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies (e.g., an anti-tryptase antibody) may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N J, 2001) and further described, e.g., in the McCafferty et al. Nature 348:552-554, 1990; Clackson et al. Nature 352: 624-628, 1991; Marks et al. J. Mol. Biol. 222: 581-597, 1992; Marks et al. in Methods in Molecular Biology 248:161-175 (Lo, ed., Human Press, Totowa, N J, 2003); Sidhu et al. J. Mol. Biol. 338(2): 299-310, 2004; Lee et al. J. Mol. Biol. 340(5): 1073-1093, 2004; Fellouse, Proc. Natl. Acad. Sci. USA 101(34):12467-12472, 2004; and Lee et al. J. Immunol. Methods 284(1-2): 119-132, 2004.

In certain phage display methods, repertoires of VH and VL genes are separately cloned by polymerase chain reaction (PCR) and recombined randomly in phage libraries, which can then be screened for antigen-binding phage as described in Winter et al. Ann. Rev. Immunol., 12: 433-455, 1994. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al. EMBO J. 12: 725-734, 1993. Finally, naive libraries can also be made synthetically by cloning unrearranged V-gene segments from stem cells, and using PCR primers containing random sequence to encode the highly variable HVR3 regions and to accomplish rearrangement in vitro, as described by Hoogenboom et al. J. Mol. Biol., 227: 381-388, 1992. Patent publications describing human antibody phage libraries include, for example: U.S. Pat. No. 5,750,373, and U.S. Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In certain aspects, an antibody provided herein (e.g., an anti-tryptase antibody) is a multispecific antibody, for example, a bispecific antibody. Multispecific antibodies are monoclonal antibodies that have binding specificities for at least two different sites. For example, with respect to anti-tryptase antibodies, in certain aspects, bispecific antibodies may bind to two different epitopes of tryptase. In certain aspects, one of the binding specificities is for tryptase and the other is for any other antigen (e.g., a second biological molecule). In some aspects, bispecific antibodies may bind to two different epitopes of tryptase. In other aspects, one of the binding specificities is for tryptase (e.g., human tryptase, e.g., human tryptase beta) and the other is for any other antigen (e.g., a second biological molecule, e.g., IL-13, IL-4, IL-5, IL-17, IL-33, IgE, M1 prime, CRTH2, or TRPA). Accordingly, the bispecific antibody may have binding specificity for tryptase and IL-13; tryptase and IgE; tryptase and IL-4; tryptase and IL-5; tryptase and IL-17, or tryptase and IL-33. In particular, the bispecific antibody may have binding specificity for tryptase and IL-13 or tryptase and IL-33. In other particular aspects, the bispecific antibody may have binding specificity for tryptase and IgE. Bispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein et al. Nature 305: 537, 1983; WO 93/08829; and Traunecker et al. EMBO J. 10: 3655, 1991), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al. Science, 229: 81, 1985); using leucine zippers to produce bispecific antibodies (see, e.g., Kostelny et al. J. Immunol., 148(5):1547-1553, 1992); using “diabody” technology for making bispecific antibody fragments (see, e.g., Hollinger et al. Proc. Natl. Acad. Sci. USA 90:6444-6448, 1993); and using single-chain Fv (scFv) dimers (see, e.g., Gruber et al. J. Immunol. 152:5368, 1994); and preparing trispecific antibodies as described, e.g., in Tutt et al. J. Immunol. 147: 60, 1991.

Engineered antibodies with three or more functional antigen binding sites, including “Octopus antibodies,” are also included herein (see, e.g., US 2006/0025576A1).

The antibody or fragment herein also includes a “Dual Acting Fab” or “DAF” comprising an antigen binding site that binds to tryptase as well as another, different antigen (see, US 2008/0069820, for example).

### Knobs-into-Holes

The use of knobs-into-holes as a method of producing multispecific antibodies is described, e.g., in U.S. Pat. No. 5,731,168, WO2009/089004, US2009/0182127, US2011/0287009, Marvin and Zhu, Acta Pharmacol. Sin. (2005) 26(6):649-658, and Kontermann (2005) Acta Pharmacol. Sin. 26:1-9. A brief nonlimiting discussion is provided below.

A “protuberance” refers to at least one amino acid side chain which projects from the interface of a first polypeptide and is therefore positionable in a compensatory cavity in the adjacent interface (i.e., the interface of a second polypeptide) so as to stabilize the heteromultimer, and thereby favor heteromultimer formation over homomultimer formation, for example. The protuberance may exist in the original interface or may be introduced synthetically (e.g., by altering nucleic acid encoding the interface). In some aspects, a nucleic acid encoding the interface of the first polypeptide is altered to encode the protuberance. To achieve this, the nucleic acid encoding at least one “original” amino acid residue in the interface of the first polypeptide is replaced with nucleic acid encoding at least one “import” amino acid residue which has a larger side chain volume than the original amino acid residue. It will be appreciated that there can be more than one original and corresponding import residue. The side chain volumes of the various amino residues are shown, for example, in Table 1 of US 2011/0287009 or Table 1 of U.S. Pat. No. 7,642,228.

In some aspects, import residues for the formation of a protuberance are naturally occurring amino acid residues selected from arginine (R), phenylalanine (F), tyrosine (Y) and tryptophan (W). In some aspects, an import residue is tryptophan or tyrosine. In some aspects, the original residue for the formation of the protuberance has a small side chain volume, such as alanine, asparagine, aspartic acid, glycine, serine, threonine, or valine. See, for example, U.S. Pat. No. 7,642,228.

A “cavity” refers to at least one amino acid side chain which is recessed from the interface of a second polypeptide and therefore accommodates a corresponding protuberance on the adjacent interface of a first polypeptide. The cavity may exist in the original interface or may be introduced synthetically (e.g., by altering nucleic acid encoding the interface). In some aspects, nucleic acid encoding the interface of the second polypeptide is altered to encode the cavity. To achieve this, the nucleic acid encoding at least one “original” amino acid residue in the interface of the second polypeptide is replaced with DNA encoding at least one “import” amino acid residue which has a smaller side chain volume than the original amino acid residue. It will be appreciated that there can be more than one original and corresponding import residue. In some aspects, import residues for the formation of a cavity are naturally occurring amino acid residues selected from alanine (A), serine (S), threonine (T), and valine (V). In some aspects, an import residue is serine, alanine, or threonine. In some aspects, the original residue for the formation of the cavity has a large side chain volume, such as tyrosine, arginine, phenylalanine, or tryptophan.

The protuberance is “positionable” in the cavity which means that the spatial location of the protuberance and cavity on the interface of a first polypeptide and second polypeptide respectively and the sizes of the protuberance and cavity are such that the protuberance can be located in the cavity without significantly perturbing the normal association of the first and second polypeptides at the interface. Since protuberances such as Tyr, Phe, and Trp do not typically extend perpendicularly from the axis of the interface and have preferred conformations, the alignment of a protuberance with a corresponding cavity may, in some aspects, rely on modeling the protuberance/cavity pair based upon a three-dimensional structure such as that obtained by X-ray crystallography or nuclear magnetic resonance (NMR). This can be achieved using widely-accepted techniques in the art.

In some aspects, a knob mutation in an IgG1 constant region is T366W. In some aspects, a hole mutation in an IgG1 constant region comprises one or more mutations selected from T366S, L368A, and Y407V. In some aspects, a hole mutation in an IgG1 constant region comprises T366S, L368A, and Y407V.

In some aspects, a knob mutation in an IgG4 constant region is T366W. In some aspects, a hole mutation in an IgG4 constant region comprises one or more mutations selected from T366S, L368A, and Y407V. In some aspects, a hole mutation in an IgG4 constant region comprises T366S, L368A, and Y407V.

### 7. Antibody Variants

In certain aspects, amino acid sequence variants of the antibodies provided herein are contemplated. For example, it may be desirable to improve the binding affinity and/or other biological properties of the antibody, such as inhibitory activity. Amino acid sequence variants of an antibody (e.g., an anti-tryptase antibody) may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

### a) Substitution, Insertion, and Deletion Variants

In certain aspects, antibody variants having one or more amino acid substitutions are provided. Sites of interest for substitutional mutagenesis include the HVRs (e.g., CDRs) and FRs. Conservative substitutions are shown in Table A under the heading of “preferred substitutions.” More substantial changes are provided in Table A under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE A

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, for example, using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196, 2008), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al. ed., Human Press, Totowa, N J, 2001). In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. HVR-H3 and HVR-L3 in particular are often targeted.

In certain aspects, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain aspects of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham et al. Science 244:1081-1085, 1989. In this method, a residue or group of target residues (e.g., charged residues such as Arg, Asp, His, Lys, and Glu) are identified and replaced by a neutral or negatively charged amino acid (e.g., Ala or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### b) Glycosylation Variants

In certain aspects, an antibody provided herein (e.g., an anti-tryptase antibody) is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, for example, Wright et al. TIBTECH 15:26-32, 1997. The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. 2003/0157108 and 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki et al. J. Mol. Biol. 336:1239-1249, 2004; Yamane-Ohnuki et al. Biotech. Bioeng. 87: 614, 2004. Examples of cell lines capable of producing defucosylated antibodies include Lec13 CHO cells deficient in protein fucosylation (Ripka et al. Arch. Biochem. Biophys. 249:533-545, 1986; US 2003/0157108; and WO 2004/056312 A1, especially at Example 11), and knockout cell lines, such as alpha-1,6-fucosyltransferase gene, FUT8, knockout CHO cells (see, e.g., Yamane-Ohnuki et al. Biotech. Bioeng. 87: 614, 2004; Kanda et al. Biotechnol. Bioeng. 94(4):680-688, 2006; and WO 2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

c) Fc region Variants

In certain aspects, one or more amino acid modifications may be introduced into the Fc region of an antibody (e.g., an anti-tryptase antibody) provided herein, thereby generating an Fc region variant. The Fc region variant may comprise a human Fc region sequence (e.g., a human IgG1, IgG2, IgG3, or IgG4 Fc region) comprising an amino acid modification (e.g., a substitution) at one or more amino acid positions.

In certain aspects, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch et al. Annu. Rev. Immunol. 9:457-492, 1991. Non-limiting examples of in vitro assays to assess ADCC activity of a molecule of interest is described in U.S. Pat. No. 5,500,362 (see, e.g., Hellstrom et al. Proc. Natl. Acad. Sci. USA 83:7059-7063, 1986 and Hellstrom et al. Proc. Natl. Acad. Sci. USA 82:1499-1502, 1985; U.S. Pat. No. 5,821,337 (see Bruggemann et al. J. Exp. Med. 166:1351-1361, 1987). Alternatively, non-radioactive assays methods may be employed (see, for example, ACTI™ non-radioactive cytotoxicity assay for flow cytometry (CellTechnology, Inc. Mountain View, CA; and Cyto Tox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, for example, in an animal model such as that disclosed in Clynes et al. Proc. Natl. Acad. Sci. USA 95:652-656, 1998. C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, e.g., Gazzano-Santoro et al. J. Immunol. Methods 202:163, 1996; Cragg et al. Blood 101:1045-1052, 2003; and Cragg et al. Blood 103:2738-2743, 2004). FcRn binding and in vivo clearance/half-life determinations can also be performed using methods known in the art (see, e.g., Petkova et al. Intl. Immunol. 18(12):1759-1769, 2006).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312; and Shields et al. J. Biol. Chem. 9(2): 6591-6604, 2001).

In certain aspects, an antibody variant comprises an Fc region with one or more amino acid substitutions which improve ADCC, e.g., substitutions at positions 298, 333, and/or 334 of the Fc region (EU numbering of residues).

In some aspects, alterations are made in the Fc region that result in altered (i.e., either improved or diminished) C1q binding and/or Complement Dependent Cytotoxicity (CDC), for example, as described in U.S. Pat. No. 6,194,551, WO 99/51642, and Idusogie et al. J. Immunol. 164: 4178-4184, 2000.

Antibodies with increased half-lives and improved binding to the neonatal Fc receptor (FcRn), which is responsible for the transfer of maternal IgGs to the fetus (Guyer et al. J. Immunol. 117:587, 1976 and Kim et al. J. Immunol. 24:249, 1994), are described in US2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan et al. Nature 322:738-40, 1988; U.S. Pat. Nos. 5,648,260 and 5,624,821; and WO 94/29351 concerning other examples of Fc region variants.

### d) Cysteine Engineered Antibody Variants

In certain aspects, it may be desirable to create cysteine engineered antibodies, for example, “thioMAbs,” in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain aspects, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### e) Antibody Derivatives

In certain aspects, an antibody provided herein may be further modified to contain additional nonproteinaceous moieties that are known in the art and readily available. The moieties suitable for derivatization of the antibody include, but are not limited to, water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, and the like.

In another aspect, conjugates of an antibody and nonproteinaceous moiety that may be selectively heated by exposure to radiation are provided. In one aspect, the nonproteinaceous moiety is a carbon nanotube (Kam et al. Proc. Natl. Acad. Sci. USA 102: 11600-11605, 2005). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Pharmaceutical Formulations

Therapeutic formulations including therapeutic agents used in accordance with the present disclosure (e.g., anti-tryptase antibodies, including any of the anti-tryptase antibodies described herein) are prepared for storage by mixing the therapeutic agent(s) having the desired degree of purity with optional pharmaceutically acceptable carriers, excipients, or stabilizers in the form of lyophilized formulations or aqueous solutions. For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol. 119, Marcel Dekker, 2002.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

The formulation herein may also contain more than one active compound, preferably those with complementary activities that do not adversely affect each other. The type and effective amounts of such medicaments depend, for example, on the amount and type of the therapeutic agent(s) present in the formulation, and clinical parameters of the subjects.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antagonist, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

### V. Articles of Manufacture and Kits

In another aspect, an article of manufacture or kit containing materials useful for the methods and uses described herein is provided. The article of manufacture may include any of the compositions (e.g., anti-tryptase antibodies or compositions thereof (e.g., pharmaceutical compositions)) provided herein. The articles of manufacture and kits may include a container and a label or package insert on or associated with the container. Suitable containers include, for example, bottles, vials, syringes, IV solution bags, and the like. The containers may be formed from a variety of materials such as glass or plastic. The container can hold a composition which is by itself or combined with another composition effective for treating, preventing and/or diagnosing the disorder (e.g., CSU (e.g., CSU refractory to antihistamines (e.g., sgH1-AHs))) and may have a sterile access port (for example the container may be an intravenous solution bag or a vial having a stopper pierceable by a hypodermic injection needle). In some aspects, at least one active agent in the composition is an anti-tryptase antibody. The label or package insert indicates that the composition is used for treating the condition of choice. The articles of manufacture or kits can include any of the compositions (e.g., pharmaceutical compositions) described herein. The article of manufacture or kit may include a pump (e.g., a patch pump), e.g., for subcutaneous administration of an anti-tryptase antibody or an antigen-binding fragment thereof. Any suitable pump described herein or known in the art may be included.

In one aspect, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in accordance with any of the methods described herein.

For example, provided is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle comprises between two and six doses, wherein a total of about 300 mg to about 21,600 mg of the anti-tryptase antibody is administered SC or IV to the patient in the dosing cycle.

For example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1) of the anti-tryptase antibody selected from 300 mg, 450 mg, 600 mg, 750 mg, 900 mg, 1350 mg, 1800 mg, or 3600 mg. In some aspects, the C1D1 is administered IV. In other aspects, the C1D1 is administered SC (e.g., by a pump (e.g., by a patch pump)).

For example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1) of the anti-tryptase antibody of 300 mg. In some aspects, the C1D1 is administered IV. In other aspects, the C1D1 is administered SC (e.g., by a pump (e.g., by a patch pump)).

In another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1) of the anti-tryptase antibody of 450 mg. In some aspects, the C1D1 is administered IV. In other aspects, the C1D1 is administered SC (e.g., by a pump (e.g., by a patch pump)).

In yet another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1) of the anti-tryptase antibody of 600 mg. In some aspects, the C1D1 is administered IV. In other aspects, the C1D1 is administered SC (e.g., by a pump (e.g., by a patch pump)).

In yet another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1) of the anti-tryptase antibody of 750 mg. In some aspects, the C1D1 is administered IV. In other aspects, the C1D1 is administered SC (e.g., by a pump (e.g., by a patch pump)).

In a further example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1) of the anti-tryptase antibody of 900 mg. In some aspects, the C1D1 is administered IV. In other aspects, the C1D1 is administered SC (e.g., by a pump (e.g., by a patch pump)).

In yet a further example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1) of the anti-tryptase antibody of 1350 mg. In some aspects, the C1D1 is administered IV. In other aspects, the C1D1 is administered SC (e.g., by a pump (e.g., by a patch pump)).

In a still further example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1) of the anti-tryptase antibody of 1800 mg. In some aspects, the C1D1 is administered IV. In other aspects, the C1D1 is administered SC (e.g., by a pump (e.g., by a patch pump)).

In another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1) of the anti-tryptase antibody of 3600 mg. In some aspects, the C1D1 is administered IV. In other aspects, the C1D1 is administered SC (e.g., by a pump (e.g., by a patch pump)).

In any of the aspects disclosed herein, the dosing cycle may further include one or more additional doses of the anti-tryptase antibody. The dosing cycle may include any suitable number of additional doses (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more additional doses) of the anti-tryptase antibody. For example, in some aspects, the dosing cycle may include a second dose (C1D2). In another example, in some aspects, the dosing cycle may include a C1D2 and a third dose (C1D3). The one or more additional doses may be equal to or unequal to the C1D1. For example, in some aspects, the dosing cycle includes a second dose (C1D2) and a third dose (C1D3) of the anti-tryptase antibody, wherein the C1D2 and the C1D3 are each equal to the C1D1. The one or more additional doses may be administered using any suitable administration route. For example, the one or more additional doses may be administered IV or SC (e.g., by a pump (e.g., by a patch pump)).

For example, in one aspect, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are selected from 300 mg, 450 mg, 600 mg, 750 mg, 900 mg, 1350 mg, 1800 mg, or 3600 mg. In some aspects, the C1D1, the C1D2, and the C1D3 are administered IV. In other aspects, the C1D1, the C1D2, and the C1D3 are administered SC (e.g., by a pump (e.g., by a patch pump)).

For example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 300 mg. In some aspects, the C1D1, the C1D2, and the C1D3 are administered IV. In other aspects, the C1D1, the C1D2, and the C1D3 are administered SC (e.g., by a pump (e.g., by a patch pump)).

In another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 450 mg. In some aspects, the C1D1, the C1D2, and the C1D3 are administered IV. In other aspects, the C1D1, the C1D2, and the C1D3 are administered SC (e.g., by a pump (e.g., by a patch pump)).

In another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 600 mg. In some aspects, the C1D1, the C1D2, and the C1D3 are administered IV. In other aspects, the C1D1, the C1D2, and the C1D3 are administered SC (e.g., by a pump (e.g., by a patch pump)).

In yet another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 750 mg. In some aspects, the C1D1, the C1D2, and the C1D3 are administered IV. In other aspects, the C1D1, the C1D2, and the C1D3 are administered SC (e.g., by a pump (e.g., by a patch pump)).

In a further example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 900 mg. In some aspects, the C1D1, the C1D2, and the C1D3 are administered IV. In other aspects, the C1D1, the C1D2, and the C1D3 are administered SC (e.g., by a pump (e.g., by a patch pump)).

In yet a further example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 1350 mg. In some aspects, the C1D1, the C1D2, and the C1D3 are administered IV. In other aspects, the C1D1, the C1D2, and the C1D3 are administered SC (e.g., by a pump (e.g., by a patch pump)).

In a still further example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 1800 mg. In some aspects, the C1D1, the C1D2, and the C1D3 are administered IV. In other aspects, the C1D1, the C1D2, and the C1D3 are administered SC (e.g., by a pump (e.g., by a patch pump)).

In another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) in a dosing regimen including a dosing cycle, wherein the dosing cycle includes a first dose (C1D1), a second dose (C1D2), and a third dose (C1D3) of the anti-tryptase antibody, wherein the C1D1, the C1D2, and the C1D3 are each 3600 mg. In some aspects, the C1D1, the C1D2, and the C1D3 are administered IV. In other aspects, the C1D1, the C1D2, and the C1D3 are administered SC (e.g., by a pump (e.g., by a patch pump)).

The doses of each dosing cycle may be administered to the subject at any suitable time interval. For example, in some aspects, the doses of the dosing cycle are administered to the subject every four weeks (Q4W).

For example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) at a dose of 300 mg SC (e.g., by a pump (e.g., by a patch pump) every four weeks (Q4W).

For example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) at a dose of 300 mg IV every four weeks (Q4W).

In another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) at a dose of 450 mg SC (e.g., by a pump (e.g., by a patch pump)) every four weeks (Q4W).

In another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) at a dose of 450 mg IV every four weeks (Q4W).

In another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) at a dose of 600 mg SC (e.g., by a pump (e.g., by a patch pump)) every four weeks (Q4W).

In another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) at a dose of 600 mg IV every four weeks (Q4W).

In yet another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) at a dose of 750 mg SC (e.g., by a pump (e.g., by a patch pump)) every four weeks (Q4W).

In a further example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) at a dose of 750 mg IV every four weeks (Q4W).

In yet another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) at a dose of 900 mg SC (e.g., by a pump (e.g., by a patch pump)) every four weeks (Q4W).

In a further example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) at a dose of 900 mg IV every four weeks (Q4W).

In yet another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) at a dose of 1350 mg SC (e.g., by a pump (e.g., by a patch pump)) every four weeks (Q4W).

In yet a further example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) at a dose of 1350 mg IV every four weeks (Q4W).

In yet another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) at a dose of 1800 mg SC (e.g., by a pump (e.g., by a patch pump)) every four weeks (Q4W).

In a still further example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) at a dose of 1800 mg IV every four weeks (Q4W).

In yet another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) at a dose of 3600 mg SC (e.g., by a pump (e.g., by a patch pump)) every four weeks (Q4W).

In another example, provided herein is a kit including any of the anti-tryptase antibodies described herein (e.g., an anti-tryptase beta antibody) and instructions to administer the anti-tryptase antibody to a patient having CSU (e.g., CSU refractory to antihistamines (e.g., sgH1-AHs)) at a dose of 3600 mg IV every four weeks (Q4W).

Each dosing cycle may have any suitable length. For example, in some aspects, each dosing cycle may have a length of about 57 days.

The doses of each dosing cycle may be administered on any suitable day(s) of the dosing cycle. For example, in some aspects, the C1D1 is administered on Day 1 of the dosing cycle, the C1D2 is administered on Day 29 (±1 day) of the dosing cycle, and the C1D3 is administered on Day 57 (±1 day) of the dosing cycle.

The dosing regimens described herein may include any suitable number of dosing cycles. For example, in some aspects, the dosing regimen includes or consists of one dosing cycle. In other aspects, the dosing regimen may include more than one dosing cycle (e.g., 2, 3, 4, 5, 6, 7, 8, 9, 10, 11, 12, 13, 14, 15, 16, 17, 18, 19, 20, or more dosing cycles).

Any suitable anti-tryptase antibody (e.g., anti-tryptase beta antibody) may be used in any of the articles of manufacture and kits described herein. For example, any of the anti-tryptase antibodies described in Section IV, Subsection A above can be used. In some aspects, the anti-tryptase antibody may be any anti-tryptase antibody described in International Patent Application Publication No. WO 2018/148585, which is incorporated herein by reference in its entirety.

For example, any of the articles of manufacture or kits may include an anti-tryptase antibody that includes one, two, three, four, five, or all six of the following complementarity determining regions (CDRs): (a) an CDR-H1 including the amino acid sequence of DYGMV (SEQ ID NO: 1); (b) an CDR-H2 including the amino acid sequence of FISSGSSTVYYADTMKG (SEQ ID NO: 2); (c) an CDR-H3 including the amino acid sequence of RNYDDWYFDV (SEQ ID NO: 3); (d) an CDR-L1 including the amino acid sequence of SASSSVTYMY (SEQ ID NO: 4); (e) an CDR-L2 including the amino acid sequence of RTSDLAS (SEQ ID NO: 5); and (f) an CDR-L3 including the amino acid sequence of QHYHSYPLT (SEQ ID NO: 6).

In any of the aspects provided herein, the antibody may include (a) a heavy chain variable (VH) domain including an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 7; (b) a light chain variable (VL) domain including an amino acid sequence having at least 90%, at least 91%, at least 92%, at least 93%, at least 94%, at least 95%, at least 96%, at least 97%, at least 98%, or at least 99% identity to the amino acid sequence of SEQ ID NO: 8; or (c) a VH domain as in (a) and a VL domain as in (b).

For example, in some aspects, the antibody may include (a) a heavy chain variable (VH) domain including an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 7. In some aspects, the VH domain includes the amino acid sequence of SEQ ID NO: 7.

In another example, in some aspects, the antibody may include (b) a light chain variable (VL) domain including an amino acid sequence having at least 90%, at least 91%, at least 92%, at least 93%, at least 94%, at least 95%, at least 96%, at least 97%, at least 98%, or at least 99% identity to the amino acid sequence of SEQ ID NO: 8. In some aspects, the VL domain includes the amino acid sequence of SEQ ID NO: 8.

In any of the aspects described herein, the VH domain may include the amino acid sequence of SEQ ID NO: 7 and the VL domain includes the amino acid sequence of SEQ ID NO: 8.

In another example, in any of the aspects described herein, the antibody may include (a) a heavy chain having at least 90%, at least 91%, at least 92%, at least 93%, at least 94%, at least 95%, at least 96%, at least 97%, at least 98%, or at least 99% identity to the amino acid sequence of SEQ ID NO: 9 and (b) a light chain having at least 90%, at least 91%, at least 92%, at least 93%, at least 94%, at least 95%, at least 96%, at least 97%, at least 98%, or at least 99% identity to the amino acid sequence of SEQ ID NO: 10. For example, in some aspects, the antibody may include (a) a heavy chain including the amino acid sequence of SEQ ID NO: 9 and (b) a light chain including the amino acid sequence of SEQ ID NO: 10.

In another example, in any of the aspects described herein, the antibody may include (a) a heavy chain having at least 90%, at least 91%, at least 92%, at least 93%, at least 94%, at least 95%, at least 96%, at least 97%, at least 98%, or at least 99% identity to the amino acid sequence of SEQ ID NO: 11 and (b) a light chain having at least 90%, at least 91%, at least 92%, at least 93%, at least 94%, at least 95%, at least 96%, at least 97%, at least 98%, or at least 99% identity to the amino acid sequence of SEQ ID NO: 10. For example, in some aspects, the antibody may include (a) a heavy chain including the amino acid sequence of SEQ ID NO: 11 and (b) a light chain including the amino acid sequence of SEQ ID NO: 10.

Any of the articles of manufacture or kits disclosed herein may include one or more additional therapeutic agents. Any suitable additional therapeutic agent(s) may be included, e.g., antihistamines (e.g., sgH1-AHs, e.g., cetirizine, levocetirizine, fexofenadine, loratadine, desloratadine, rupatadine, bilastine, or a combination thereof), an anti-IgE antibody (e.g., omalizumab (XOLAIR®) or ligelizumab (also known as QGE031)), a Syk inhibitor (e.g., GSK2646264), an anti-Siglec-8 antibody (e.g., lirentelimab (also known as AK002)), a BTK inhibitor (e.g., ibrutinib or fenebrutinib), an LTRA, a CRTh2 antagonist (e.g., AZD1981), an IL-1 antagonist (e.g., an anti-IL-1 antibody, e.g., an anti-IL-1β antibody, e.g., canakinumab), an IL-5 antagonist (e.g., an anti-IL-5 antibody (e.g., mepolizumab) or an anti-IL-5 receptor alpha antibody (e.g., benralizumab)), dapsone, cyclosporin A, or any combination thereof. Exemplary additional therapeutic agents for treatment of CSU are described, e.g., in Min et al. Allergy Asthma Immunol. Res. 11(4):470-481, 2019. The one or more additional therapeutic agents may be standard of care for CSU. Any suitable standard of care for CSU may be included, e.g., sgH1-AHs. A person of skill in the art will be able to select a suitable standard of care as appropriate.

### EXAMPLES

The following examples are provided to illustrate, but not to limit the presently claimed invention.

### Example 1: A Phase II, Multicenter, Randomized, Double-Blind, Placebo-Controlled Pilot and Dose-Ranging Study of MTPS9579A in Participants with Refractory Chronic Spontaneous Urticaria

### A. Objectives and Endpoints

This example describes a Phase II, randomized, double-blind, placebo-controlled pilot and dose-ranging study to evaluate the efficacy, safety, and pharmacokinetics of MTPS9579A compared with placebo in patients with chronic spontaneous urticaria (CSU) refractory to antihistamines (up to 4 times the approved dose per local treatment guidelines). Specific objectives and corresponding endpoints for the study are outlined below.

i. Primary Efficacy Objective

The primary efficacy objective for this study is to evaluate the efficacy of MTPS9579A compared with placebo on the basis of the following endpoint:
Change from baseline in the Urticaria Activity Score summed over 7 days (UAS7) at Week 12
   ii. Secondary Efficacy Objective

The secondary efficacy objective for this study is to evaluate the efficacy of MTPS9579A compared with placebo on the basis of the following endpoints:
Proportion of patients with well-controlled urticaria (UAS7≤6) at Week 12
Proportion of patients who achieve complete response (UAS7=0) at Week 12
   iii. Exploratory Efficacy Objective

The exploratory efficacy objective for this study is to evaluate the efficacy of MTPS9579A compared with placebo on the basis of the following endpoints:
Change from baseline in the UAS7 at Weeks 4 and 8
Change from baseline in the Itch-Severity Score summed over 7 days (ISS7) at Weeks 4, 8, and 12
Change from baseline in the Hives-Severity Score summed over 7 days (HSS7) at Weeks 4, 8, and 12
Proportion of patients who achieve complete response (UAS7=0) at Weeks 4 and 8
Proportion of patients achieving the minimally important difference (MID) in UAS7 at Week 12 (reduction from baseline ≥11 points)
Proportion of patients achieving the MID in the ISS7 at Week 12 (reduction from baseline ≥5 points)
Proportion of patients achieving the MID in the HSS7 at Week 12 (reduction from baseline ≥6 points)
Time to achieving the MID in UAS7 (reduction from baseline ≥11 points)
Time to achieving the MID in the ISS7 (reduction from baseline ≥5 points)
Time to achieving the MID in the HSS7 (reduction from baseline ≥6 points)
Change from baseline in the Urticaria Control Test (UCT) score at Week 12
Proportion of patients with well-controlled urticaria (UCT score ≥12) at Week 12
Change from baseline in the Angioedema Activity Score summed over 7 days (AAS7) at Weeks 4, 8, and 12
Proportion of patients achieving the MID in the AAS7 at Week 12 (reduction from baseline ≥8 points)
Time to achieving the MID in AAS7 (reduction from baseline ≥8 points)
Change from baseline in the Angioedema Control Test (AECT) score at Week 12
Proportion of patients with well-controlled angioedema (AECT score ≥10) at Week 12
Number of angioedema episodes
Use of rescue medication
   iv. Safety Objective

The safety objective for this study is to evaluate the safety of MTPS9579A compared with placebo on the basis of the following endpoints:
Incidence and severity of adverse events, with severity determined according to the Division of AIDS Table for Grading the Severity of Adult and Pediatric Adverse Events (DAIDS AE Grading Table)
Change from baseline in targeted vital signs, physical examination findings, clinical laboratory test results, and ECG parameters
   v. Pharmacokinetic Objectives

The pharmacokinetic (PK) objective for this study is to characterize the PK profile of MTPS9579A on the basis of the following endpoint:
Serum concentration of MTPS9579A at specified timepoints

The exploratory PK objectives for this study are as follows:
To evaluate potential relationships between drug exposure and the efficacy and safety of MTPS9579A on the basis of the following endpoints:
   Relationship between serum concentration or PK parameters of MTPS9579A and efficacy endpoints
   Relationship between serum concentration or PK parameters of MTPS9579A and safety endpoints
      vi. Immunogenicity Objectives

The immunogenicity objective for this study is to evaluate the immune response to MTPS9579A on the basis of the following endpoint:
Prevalence of anti-drug antibodies (ADAs) at baseline and incidence of ADAs during the study

The exploratory immunogenicity objective for this study is to evaluate potential effects of ADAs on the basis of the following endpoint:
·Relationship between ADA status and efficacy, safety, or PK endpoints
   vii. Biomarker Objective

The exploratory biomarker objective for this study is to identify and/or evaluate biomarkers that are predictive of response to MTPS9579A (i.e., predictive biomarkers), can provide evidence of MTPS9579A activity (i.e., pharmacodynamic biomarkers), or can increase the knowledge and understanding of disease biology and drug safety, on the basis of the following endpoints:
Relationship between biomarkers in serum, urine, and nasal mucosal lining fluid and efficacy, safety, PK, immunogenicity, or other biomarker endpoints
Change from baseline in biomarker levels in serum, urine, and nasal mucosal lining fluid samples

### B. Study Design

This is a Phase II, multicenter, randomized, double-blind, placebo-controlled pilot and dose-ranging study of MTPS9579A as an add-on therapy for the treatment of adult patients 18-75 years old who have been diagnosed with CSU and remain symptomatic despite treatment with second-generation H1 antihistamines (sgH1-AHs) (including at doses up to 4 times the approved dose level per local treatment guidelines). Patients with prior anti-IgE experience are permitted, but current use is not permitted, and enrollment of primary non-responders is capped.

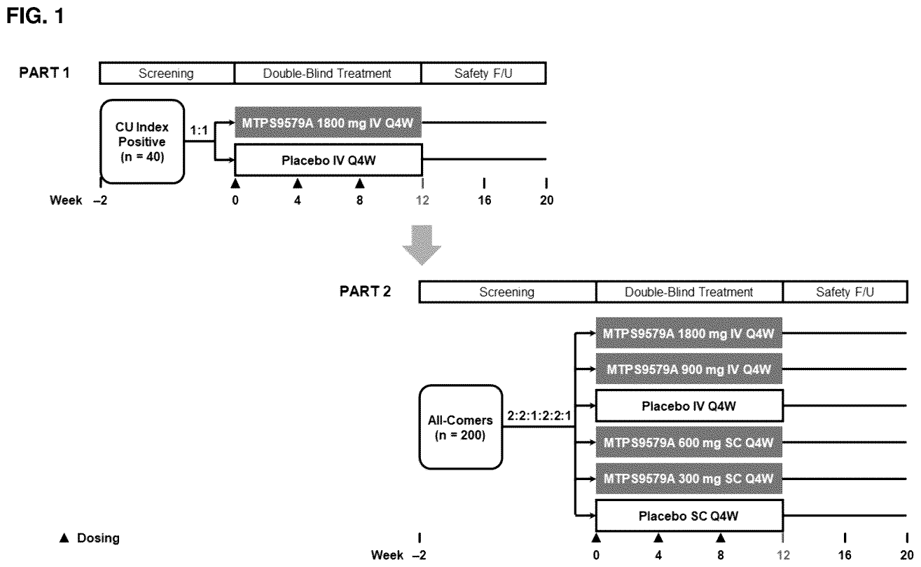

This study comprises two parts (see FIG. 1). Parts 1 and 2 of this study randomize an estimated 240 patients at approximately 95 sites globally. In Part 1, eligible Chronic Urticaria Index (CU Index®)-positive patients (n=40) are randomized in a 1:1 ratio to receive MTPS9579A (1800 mg IV Q4W) or placebo. On the basis of positive results from Part 1, a dose-ranging Part 2 may be opened to eligible patients irrespective of CU Index status (all-comers; n=200), where patients are randomized in a 2:2:1:2:2:1 ratio to receive one of four MTPS9579A doses or placebo (1800 mg IV Q4W; 900 mg IV Q4W; placebo IV Q4W; 600 mg SC Q4W; 300 mg SC Q4W; placebo SC Q4W). Other than the evaluation of different doses and study populations based on CU Index, both study parts are conducted identically and include the following:
Screening Period. Up to 2 weeks to establish eligibility, including stable use of background sgH1-AH therapy and baseline symptom scores
Double-Blind Treatment Period. From Week 0 to Week 12, with administration of MTPS9579A by IV infusion or SC injection, as indicated, at the randomization visit (Week 0), Week 4, and Week 8
Safety Follow-Up Period. From Week 12 to Week 20

During the screening period, patients must have documented treatment with a regimen that includes a standard-of-care background sgH1-AH for CSU at Day −14 and for at least the 3 consecutive days immediately prior to Day −14 (see below for a list of sgH1-AHs allowed for use in this study). All patients are maintained on stable doses of sgH1-AHs (background therapy) throughout the study from screening through safety follow-up.

Eligibility criteria are listed below in the Materials and Methods section. Of note, to be eligible for randomization in either study part, patients must meet the following criteria for the 7 days prior to randomization: (1) 7 consecutive days of entries in an electronic diary (eDiary), and (2) a UAS7 symptom score of ≥16 (range, 0-42). Only in exceptional circumstances, when information concerning eligibility is outstanding (e.g., delayed laboratory results), is a longer screening period permitted, up to 3 additional business days.

During the treatment period, twice-daily assessment of CSU-related symptoms and use of rescue therapy continue to be performed at home and recorded in the eDiary. Additional assessments of CSU signs and symptoms are performed at scheduled site visits using the UCT. All patients undergo PK, biomarker, and ADA sampling.

During the 8-week safety follow-up period, additional safety and efficacy data are collected and further characterization of the pharmacokinetics and pharmacodynamics of MTPS9579A are performed. No study treatment is administered during follow-up; however, to reduce patient dropout and thus improve safety evaluation, patients may add up to one additional sgH1-AH therapy in case of worsened symptoms (see Study Treatment section below).

Patients are permitted to use such rescue therapy as needed throughout the entirety of the study.

Patients who do not meet the criteria for participation in this study (screen failure) may qualify for one re-screening opportunity (for a total of two screenings per patient) at the investigator's discretion, after discussion with the Medical Monitor. In addition, subjects who do not qualify for Part 1 because of CU Index® status (i.e., those with a negative CU Index®) may re-screen for Part 2 of the study.

The end of this study is defined as the date when the last patient, last visit (LPLV), occurs or safety follow-up is received from the last patient, whichever occurs later. The end of the study is expected to occur 20 weeks after the last patient is enrolled.

The total length of the study, from screening of the first patient to the end of the study, is expected to be approximately 48 weeks for Part 1 and 96 weeks for Part 2.

### C. Materials and Methods

i. Inclusion Criteria
Patients are required to meet the following criteria for study entry:
Age 18-75 years at time of signing Informed Consent Form
Ability to comply with the study protocol, including stable background therapy, visit schedules, and daily use of the eDiary, in the investigator's judgment
Body mass index (BMI) of 18-38 kg/m² and weight ≥40 kg at screening
Diagnosis of CSU refractory to sgH1-AHs at the time of randomization, as defined by all of the following criteria:
   CSU diagnosis for ≥6 months
   Presence of itch and hives for >6 consecutive weeks at any time prior to enrollment despite current use of sgH1-AHs, consistent with standard of care (i.e., up to 4 times the approved dose per local treatment guidelines) during this time period
   Receipt of stable doses of sgH1-AHs, consistent with standard-of-care therapy (i.e., up to 4 times the approved dose per local treatment guidelines) for CSU, starting at least 3 consecutive days immediately prior to the screening visit through Day 1, and agreement to continue stable use through safety follow-up and document current use
   UAS7≥16 during the 7 days prior to randomization (Day 1)
For Part 1 only: CU Index® positivity at screening, as confirmed by central laboratory
   Note: There is no requirement with regard to CU Index® for Patients in Part 2.
Demonstrated compliance with required use of the UPDD for the duration of the study
   Completion of 7 days of the UPDD entries in the 7 days prior to randomization (7 of 7 days must be completed (i.e., one of the two daily entries every day) with up to 2 non-consecutive entries missed)
For patients on H2 antihistamines or leukotriene receptor antagonists (LTRAs) for non-CSU indications (i.e., gastroesophageal reflux disease (GERD) or asthma), treatment at a stable dose for ≥2 weeks prior to screening, with no anticipated changes throughout duration of study, including the screening period
   ii. Exclusion Criteria

Patients who meet any of the following criteria are excluded from study entry:
Previous participation in a clinical trial of MTPS9579A
   Note: Patients enrolled in Part 1 of this study are not eligible for Part 2 of this study, even if they may have been randomized to the placebo group.
Chronic urticaria with known cause (e.g., inducible) or other diseases with symptoms of urticaria or angioedema (urticarial vasculitis, urticaria pigmentosa, mastocytosis, hereditary or acquired angioedema)
   Note: Patients may be eligible if their CSU is comorbid with a physical or inducible urticaria (e.g., symptomatic dermatographism).
Skin disease associated with chronic itching (e.g., atopic dermatitis or psoriasis)
Uncontrolled disease (e.g., asthma or inflammatory bowel disease) where flares are commonly treated with systemic (oral or parenteral) corticosteroids
History or evidence of any clinically significant medical condition/disease (e.g., psychiatric, neurologic, cardiovascular, renal, hepatic, gastrointestinal, endocrine, or autoimmune) or abnormalities in laboratory tests that, in the investigator's judgment, precludes the patient's safe participation and completion of the study, or interferes with the conduct and interpretation of the study
   Patients with well-controlled comorbid disease on a stable treatment regimen for 4 weeks prior to screening are eligible for the study.
   Any items that are cause for uncertainty must be discussed with the Medical Monitor.
History or evidence of substance abuse that, in the investigator's judgment, would affect the patient's ability to participate in the study, pose a risk to patient safety, interfere with the conduct of the study, or have an impact on the study results
History of anaphylaxis without clearly identifiable avoidable antigen (e.g., due to food allergy)
History of anaphylaxis to any biologic therapy for any indication
History of documented immune complex disease (Type Ill hypersensitivity reactions) to monoclonal administration
Known sensitivity to any of the active substances or their excipients to be administered during dosing
Treatment with anti-IgE antibody or other monoclonal antibody therapies used to treat CSU within 3 months prior to screening
Use of a first-generation H1 antihistamine (e.g., diphenhydramine, hydroxyzine, promethazine, chlorpheniramine) within 2 weeks prior to screening
Use of a non-biologic investigational drug or participation in an investigational study with a non-biologic drug within 30 days or 5 drug-elimination half-lives, whichever is longer, prior to study drug administration on Day 1
Use of a biologic investigational therapy or participation in an investigational study with a biologic therapy within 90 days or 5 drug-elimination half-lives, whichever is longer, prior to study drug administration on Day 1
Routine (daily or every other day during 5 or more consecutive days) doses of any of the following medications within 30 days prior to screening: systemic or cutaneous (topical) corticosteroids (prescription or over the counter), hydroxychloroquine, methotrexate, cyclosporine, tacrolimus, or cyclophosphamide
   Inhaled asthma controllers, including inhaled corticosteroids, are permitted during the study.
Prior utilization of IV corticosteroids for treatment of laryngeal angioedema
IVIG or plasmapheresis within 30 days prior to screening
Initiation of or change in allergen immunotherapy within 3 months prior to screening or during the screening period, or anticipated need during the course of the study
Treatment with immunoglobulin or blood products within 4 weeks prior to screening or during the screening period, or anticipated need during the course of the study
Treatment with intra-articular corticosteroids within 4 weeks prior to screening or during the screening period, or anticipated need during the course of the study
Treatment with mast cell stabilizers (e.g., cromolyn) within 2 weeks prior to screening or during the screening period, or anticipated need during the course of the study
Treatment with homeopathic medications, herbal medications, acupuncture, or hypnosis for treatment of allergic disease within 2 weeks prior to screening or during the screening period, or anticipated need during the course of the study
History of live, attenuated vaccine within 4 weeks prior to randomization, or requirement to receive these vaccinations at any time during study drug treatment, unless the vaccine is deemed medically necessary and no inactivated vaccine alternatives are available
   Most authorized vaccines for SARS-CoV-2, including those delivered by non-replicating viral vectors are acceptable. However, this is a rapidly evolving field, and patients who become eligible for a live SARS-CoV-2 vaccine (when available) prior to or during screening should not delay vaccination in order to enroll in the study and can re-screen 4 weeks after completing the vaccination schedule.
Major surgery within 8 weeks prior to screening, or planned surgery prior to end of study (20 weeks after randomization)
Planned hospitalization for any medical condition prior to the end of study (20 weeks after randomization)
Myocardial infarction, unstable angina pectoris, or stroke within 12 months prior to screening
Any chronic heart failure exacerbation within 12 months prior to screening, or at risk for heart failure exacerbation in the investigator's opinion
History or presence of an abnormal ECG that is clinically significant in the investigator's opinion, including complete left bundle branch block, second- or third-degree atrioventricular heart block, or evidence of prior myocardial infarction
QT interval corrected through use of Fridericia's formula (QTcF)>450 ms, if patient is male, or QTcF>470, if patient is female, demonstrated by at least two ECGs>30 minutes apart
Active malignancy or history of malignancy within 5 years of screening, except for appropriately treated non-melanoma skin carcinoma, cervical carcinoma in situ, breast ductal carcinoma in situ, or Stage I uterine cancer
Hemoglobin A1c (HbA1c)>8.5% at screening or any other clinically significant finding that, in the opinion of the investigator, may define uncontrolled diabetes
Positive hepatitis C virus (HCV) antibody at screening, unless HCV RNA<15 IU/mL (or undetectable) at screening with successful completion of HCV antiviral treatment at least 6 months prior to screening
Hepatitis B infection, as evidenced by either of the following:
   Positive hepatitis B surface antigen (HBsAg) at screening
   Positive hepatitis B core antibody (HBcAb) in conjunction with a positive hepatitis B virus (HBV) DNA test at screening
Positive HIV antibody at screening
History of any known immunodeficiency disorder
Acute infection requiring either surgical intervention (e.g., drainage) or medical therapy (e.g., antibiotics) within 4 weeks prior to screening
   Testing for COVID-19 should be performed in accordance to local practice; a history of a positive test within 4 weeks prior to screening is exclusionary.
Active parasitic infection within 6 months prior to screening, including current or recent helminth infection
   iii. Study Treatment

The investigational medicinal products (IMPs) for this study are MTPS9579A and placebo. Background and rescue sgH1-AHs are considered non-investigational medicinal products (NIMPs).

### Investigational Medicinal Products

### MTPS9579A and Placebo

### Intravenous Administration

The IV doses and regimens of IMP are the following: 1800 mg IV Q4W, 900 mg IV Q4W, and placebo IV Q4W. For IV administration, the MTPS9579A or matched placebo dose is prepared by diluting study drug or placebo with saline. For patients who experience mild infusion-related signs or symptoms (Grade ≤2), the infusion time may be modified. For patients with Grade 3 or higher infusion-related signs or symptoms requiring treatment, the infusion should be discontinued, and the Sponsor should be notified immediately. Patients should not be medicated or premedicated in order to tolerate IV administration of study drug. Any changes in the rate of infusion or disruptions of infusion are carefully documented. Infusions of MTPS9579A or placebo are administered in a monitored setting where there is immediate access to trained personnel and adequate equipment and medicine to manage potentially serious reactions.

### Subcutaneous Administration

The SC doses and regimens of IMP are the following: 600 mg SC Q4W, 300 mg SC Q4W, and placebo SC Q4W. For SC administration, undiluted MTPS9579A (150 mg/mL concentration) or matched placebo is injected using syringes provided by the study site. All study drug is injected in the abdomen, if possible. Use of an alternate injection site may be considered if needed to ensure SC rather than IM injection. The preferred alternate injection site is the back of the upper arm. The exact location of the injection should be carefully documented (e.g., 3 o'clock position, 5 cm from the edge of the navel). Injections of MTPS9579A or placebo are administered in a monitored setting where there is immediate access to trained personnel and adequate equipment and medicine to manage potentially serious reactions.

### Non-Investigational Medicinal Products

### Background Therapy for CSU

All patients are on stable background therapy for CSU during the screening, treatment, and follow-up periods. Background therapy is defined as a sgH1-AH CSU treatment regimen that is consistent with EAACI/GA²LEN/EDF/WAO (European Academy of Allergology and Clinical Immunology (EAACI), Global Allergy and Asthma European Network (GA²LEN), European Dermatology Forum (EDF) and World Allergy Organization (WAO)) guidelines (i.e., up to 4 times the approved dose per local treatment guidelines). Patients should remain on a stable sgH1-AH regimen throughout the study period. Therapies used for the treatment of CSU prior to enrollment are collected as part of the patient's medical history.

As background therapy, the following sgH1-AH medications are allowed: cetirizine, 10-40 mg QD; levocetirizine, 5-20 mg QD; fexofenadine, 180-720 mg QD; loratadine, 10-40 mg QD; desloratadine 5-20 mg QD; rupatadine, 10-40 mg QD; or bilastine, 20-80 mg QD.

### Rescue Therapy

In addition to their daily background therapy, for the duration of the study, all patients are able to use a single approved dose of loratadine (10 mg maximum) or cetirizine (10 mg maximum) within a 24-hour period as rescue medication if symptoms worsen during the screening, treatment, or follow-up periods. If a patient needs rescue therapy and is already on background treatment with cetirizine or loratadine, the patient may receive 10 mg more of the same drug only if the total daily dose remains below 4 times the approved dose. Otherwise, the alternate rescue medication may be used. Patients should record the use of this medication in their eDiary. Although prohibited, the use of other rescue medications (e.g., corticosteroids or H2 antihistamines) should also be reported.

iv. Study Assessments

Patient-reported outcome (PRO) instruments are completed to assess the treatment benefit of MTPS9579A. PRO data are collected via electronic questionnaires using the following instruments: the UPDD (composed of the Urticaria Activity Score (UAS), the Angioedema Activity Score (AAS), and additional items), the Urticaria Control Test (UCT), and the Angioedema Control Test (AECT).

PRO instruments are self-administered at specified timepoints during the study, with the UCT and AECT completed at the clinic (or via mobile nursing (MN)) and the eDiary completed at home. At the clinic (or via MN), the UCT and AECT are administered before the patient receives any information on disease status, prior to the performance of non-PRO assessments, and prior to the administration of study treatment, unless otherwise specified.

The UPDD is composed of the UAS questionnaire, used to calculate the UAS7; the AAS, used to calculate the AAS7; and additional items regarding largest hive size, sleep interference, activity interference, rescue medication use, number of calls to the doctor or nurse practitioner, and background therapy compliance.

The eDiary is to be completed twice per day (morning and evening) by the patient for the duration of the study. Note that itch severity, number of hives, and largest hive items are asked twice daily while other items are asked once, per eDiary programming. The eDiary is provided to patients at the Day −14 visit.

The UAS is a composite, eDiary-recorded score with numeric severity intensity ratings (0=none to 3=intense/severe) for (a) the number of wheals (hives) and (b) the intensity of the pruritus (itch) over the past 12 hours (twice daily). The daily UAS is calculated as the average of the morning and evening scores. The baseline UAS7 is calculated as the sum of daily UAS values over the week (7 days) prior to Day 1. The UAS7 is calculated as the daily UAS, which is the composite score of the number of wheals and the intensity of pruritus, summed over 7 days. The maximum UAS7 value is 42; the number of wheals/hives and the intensity of the itch/pruritus are graded. Weekly scores for wheals/hives (HSS7) and pruritus/itch severity (ISS7) are calculated as the summed mean daily scores for each component (range, 0-21).

The AAS is a composite, eDiary-recorded score gated by a question asking respondents whether they experienced a swelling episode in the last 24 hours. If answered affirmatively, follow-up questions are asked to indicate 8-hour periods of angioedema presence and rate levels of discomfort severity, interference with performance of daily activities, adverse impact on appearance, and overall severity of the episode. The maximum daily score range is 0-15, which is summed over 7 days to calculate the AAS7 (range, 0-105). Higher scores indicate worse angioedema; the MID is 8 points (Weller at al. Allergy. 68: 1185-1192, 2013).

The UCT is a 4-item questionnaire to assess disease activity. The recall period is 4 weeks and the score range is 0-16, with higher scores indicating greater disease activity/lower disease control. The MID is 2.8 points (Kulthanan et al. Health Qual Life Outcomes. 14: 1-9, 2016). The questionnaire is filled out at baseline (Day 1) and at specified timepoints.

The AECT is a 4-item questionnaire to assess angioedema-specific disease activity. The recall period is 4 weeks and the score range is 0-16, with higher scores indicating greater disease activity/lower disease control. Scores ≥10 indicate well-controlled angioedema (Weller et al. J Allergy Clin Immunol. 8: 2050-2057, 2020). The questionnaire is filled out at baseline (Day 1) and at specified timepoints.

The following samples are sent to one or several central laboratories or to the Sponsor or a designee for analysis:
Serum samples for determination of CU Index®
Serum, blood, urine, and nasal mucosal lining fluid samples for exploratory research on biomarkers
Serum samples for PK analysis
Serum samples for immunogenicity analysis
   Blood and urine sample collection may be performed by an MN professional.

Exploratory biomarker research may include, but is not limited to CU Index®, active tryptase, total tryptase, urea, and inflammatory lipids. Research may involve extraction of DNA and genomic profiling through use of single nucleotide polymorphisms and next-generation sequencing (NGS) of a comprehensive panel of genes. Genomic research is aimed at exploring inherited characteristics. NGS methods may include whole genome sequencing (WGS) of blood samples.

v. Statistical Analysis

The primary and secondary efficacy analyses are based on a modified intent-to-treat (mITT) approach. All patients who received at least one dose of study drug are included in the mITT population, with patients grouped according to the treatment assigned at randomization. Safety analyses are conducted on the safety-evaluable population, defined as all patients who received at least one dose of study drug, with patients grouped according to the actual treatment received.

For each part, the final analysis of data from the 12-week, placebo-controlled period is performed when the following two criteria have been met: 1) all patients in the cohort have either completed the Day 85 (Week 12) visit or discontinued from the placebo-controlled period prematurely, and 2) all data from the placebo-controlled period in the cohort are in the database and have been cleaned and verified. Patients and study site personnel remain blinded to the individual treatment assignment until after the study is completed (i.e., after all patients in both parts have either completed the safety follow-up period or discontinued early from the study), the database is locked, and the study analyses are final for both parts.

The focus of the trial is estimation and generation of hypotheses to be confirmed in future trials; therefore, Type I error control is not addressed.

### Determination of Sample Size

### Part 1

The purpose of Part 1 of the study is to evaluate the efficacy of MTPS9579A 1800 mg IV Q4W compared with placebo in improving the UAS7. Point and interval estimates of the change from baseline in the UAS7 within each treatment group as well as of the difference in change from baseline in the UAS7 between treatment groups are presented. Part 1 enrolls approximately 40 patients. Patients are randomized in a 1:1 ratio to receive treatment with either MTPS9579A or placebo. The sample size of approximately 20 patients in each arm provides approximately 80% power to detect an 11-point difference in the UAS7 change from baseline at Week 12 between treatment groups under the following assumptions:
The absolute change from baseline at Week 12 is normally distributed, with a standard deviation of 13.
The two-sided a is 0.10.
The dropout rate at Week 12 is 10%, leading to a 10% loss of information.

### Part 2

The purpose of Part 2 of the study is estimation and hypothesis generation regarding the dose-ranging effects of MTPS9579A compared with placebo in improving the UAS7. Point and interval estimates of the change from baseline in the UAS7 within each treatment group as well as of the difference in change from baseline in the UAS7 between treatment groups versus placebo are presented. Part 2 enrolls approximately 200 patients. Patients are randomly allocated in a 2:2:1:2:2:1 ratio to receive treatment with one of four dose levels of MTPS9579A or placebo. The two placebo arms are pooled for analysis. The sample size of approximately 40 in each MTPS9579A arm and the pooled placebo arm provides approximately 97% power to detect an 11-point difference in the UAS7 change from baseline at Week 12 between one MTPS9579A arm and the pooled placebo arm, under the following assumptions:
The absolute change from baseline at Week 12 is normally distributed, with a standard deviation of 13.
The two-sided a is 0.10.
The dropout rate at Week 12 is 10%, leading to a 10% loss of information.

### Efficacy Analyses

Statistical analysis is conducted for each part separately. Statistical testing is viewed as hypothesis generating rather than confirmatory, and no multiplicity adjustments are performed to control overall Type I error.

Continuous longitudinal efficacy endpoints are analyzed using a mixed model for repeated measures (MMRM) and descriptive statistics as appropriate. Time-to-event endpoints are analyzed using a Cox proportional hazards model. Categorical endpoints are analyzed using an appropriate statistical method, such as Cochran-Mantel-Haenszel test or Fisher's exact test.

Unless otherwise noted, all analyses of efficacy outcome measures in Part 1 are adjusted by prior anti-IgE experience (naïve vs. experienced), CSU severity (moderate activity urticaria (UAS7 16-27) vs. severe activity urticaria (UAS7 28-42)); all analyses of efficacy outcome measures in Part 2 are adjusted by prior anti-IgE experience (naïve vs. experienced), CSU severity (moderate activity urticaria (UAS7 16-27) vs. severe activity urticaria (UAS7 28-42)), and CU Index® (positive vs. negative).

The primary endpoint is analyzed using a MMRM model. Additional model covariates include baseline UAS7 and its interaction with visit.

### Safety Analyses

The safety analysis population consists of all randomized patients who received at least one dose of study drug, with patients grouped according to treatment received. Summaries of adverse events, serious adverse events, deaths, adverse events of special interest, adverse events that lead to discontinuation, ECG findings, laboratory test results, and vital sign measurements are to be presented.

### Pharmacokinetic Analyses

The PK analysis population consists of patients who received MTPS9579A with at least one post-treatment measurement of serum concentration. Individual and mean serum MTPS9579A concentration versus time data are tabulated and plotted by dose level. Additional PK and PK/PD analyses are conducted as appropriate.

### Immunogenicity Analyses

Administration of MTPS9579A, a monoclonal anti-tryptase antibody, may result in an immune response in patients resulting in ADAs. Serum samples are collected at defined intervals to monitor the development of ADAs. The immunogenicity analysis population consists of all patients with at least one ADA assessment. Patients are grouped according to treatment received or, if no treatment is received prior to study discontinuation, according to treatment assigned.

### Biomarker Analyses

Biomarkers are assessed to determine pharmacological activity and mechanism of action of MTPS9579A. Data are summarized by absolute levels of the biomarker, as well as absolute and relative changes from randomization (defined as predose), for each treatment group. Additional PD analyses are conducted as appropriate.

Potential predictive biomarkers of MTPS9579A response are assessed in primary and key secondary endpoints to assess if a subset of patients derives enhanced clinical benefit from MTPS9579A. Predictive biomarker candidates include, but are not limited to, CU Index®, baseline serum total tryptase level, and germline mutations in the genes encoding tryptase (TPSAB1 and TPSB2) (see, e.g., U.S. Patent Application Publication No. US 2020/0377953, which is incorporated by reference herein in its entirety).

### Interim Analysis

Given the hypothesis-generating nature of this study, the Sponsor may choose to conduct one interim efficacy analysis. The decision to conduct an optional interim analysis and the timing of the analysis is documented in the Sponsor's trial master file prior to the conduct of the interim analysis.

### Other Aspects

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method of treating a patient having chronic spontaneous urticaria (CSU), the method comprising administering to a patient having CSU an anti-tryptase beta antibody in a dosing regimen comprising a dosing cycle, wherein the dosing cycle comprises a first dose (C1D1) of the anti-tryptase beta antibody selected from 300 mg subcutaneously (SC), 600 mg SC, 900 mg intravenously (IV), or 1800 mg IV, wherein the anti-tryptase beta antibody comprises the following six complementarity determining regions (CDRs):
(a) an CDR-H1 comprising the amino acid sequence of DYGMV (SEQ ID NO: 1);
(b) an CDR-H2 comprising the amino acid sequence of FISSGSSTVYYADTMKG (SEQ ID NO: 2);
(c) an CDR-H3 comprising the amino acid sequence of RNYDDWYFDV (SEQ ID NO: 3);
(d) an CDR-L1 comprising the amino acid sequence of SASSSVTYMY (SEQ ID NO: 4);
(e) an CDR-L2 comprising the amino acid sequence of RTSDLAS (SEQ ID NO: 5); and
(f) an CDR-L3 comprising the amino acid sequence of QHYHSYPLT (SEQ ID NO: 6).

2. The method of claim 1, wherein the antibody comprises (a) a heavy chain variable (VH) domain comprising an amino acid sequence having at least 90%, at least 95%, or at least 99% sequence identity to the amino acid sequence of SEQ ID NO: 7; (b) a light chain variable (VL) domain comprising an amino acid sequence having at least 90%, at least 95%, or at least 99% identity to the amino acid sequence of SEQ ID NO: 8; or (c) a VH domain as in (a) and a VL domain as in (b).

3. The method of claim 2, wherein the VH domain comprises the amino acid sequence of SEQ ID NO: 7.

4. The method of claim 2, wherein the VL domain comprises the amino acid sequence of SEQ ID NO: 8.

5. The method of claim 2, wherein the VH domain comprises the amino acid sequence of SEQ ID NO: 7 and the VL domain comprises the amino acid sequence of SEQ ID NO: 8.

6. The method of any one of claims 1-5, wherein the antibody comprises (a) a heavy chain comprising the amino acid sequence of SEQ ID NO: 9 and (b) a light chain comprising the amino acid sequence of SEQ ID NO: 10.

7. The method of any one of claims 1-5, wherein the antibody comprises (a) a heavy chain comprising the amino acid sequence of SEQ ID NO: 11 and (b) a light chain comprising the amino acid sequence of SEQ ID NO: 10.

8. The method of any one of claims 1-7, wherein the C1D1 is 300 mg SC.

9. The method of any one of claims 1-7, wherein the C1D1 is 600 mg SC.

10. The method of any one of claims 1-7, wherein the C1D1 is 900 mg IV.

11. The method of any one of claims 1-7, wherein the C1D1 is 1800 mg IV.

12. The method of any one of claims 1-11, wherein the dosing cycle further comprises a second dose (C1D2) and a third dose (C1D3) of the anti-tryptase beta antibody, wherein the C1D2 and the C1D3 are each equal to the C1D1.

13. The method of claim 12, wherein the doses of the dosing cycle are administered to the subject every four weeks (Q4W).

14. The method of claim 12 or 13, wherein the dosing cycle has a length of about 57 days.

15. The method of claim 14, wherein the C1D1 is administered on Day 1 of the dosing cycle, the C1D2 is administered on Day 29 (±1 day) of the dosing cycle, and the C1D3 is administered on Day 57 (±1 day) of the dosing cycle.

16. The method of any one of claims 12-15, wherein the dosing regimen consists of one dosing cycle.

17. A method of treating a patient having CSU, the method comprising administering to a patient having CSU an anti-tryptase beta antibody in a dosing regimen comprising a dosing cycle, wherein the dosing cycle comprises between two and six doses, wherein a total of about 300 mg to about 21,600 mg of the anti-tryptase antibody is administered SC or IV to the patient in the dosing cycle, and wherein the anti-tryptase beta antibody comprises the following six CDRs:
(a) an CDR-H1 comprising the amino acid sequence of DYGMV (SEQ ID NO: 1);
(b) an CDR-H2 comprising the amino acid sequence of FISSGSSTVYYADTMKG (SEQ ID NO: 2);
(c) an CDR-H3 comprising the amino acid sequence of RNYDDWYFDV (SEQ ID NO: 3);
(d) an CDR-L1 comprising the amino acid sequence of SASSSVTYMY (SEQ ID NO: 4);
(e) an CDR-L2 comprising the amino acid sequence of RTSDLAS (SEQ ID NO: 5); and
(f) an CDR-L3 comprising the amino acid sequence of QHYHSYPLT (SEQ ID NO: 6).

18. A method of treating a patient having CSU, the method comprising administering to a patient having CSU an anti-tryptase beta antibody in a dosing regimen comprising a dosing cycle, wherein the dosing cycle comprises administering the anti-tryptase beta antibody at a dose selected from 300 mg SC, 600 mg SC, 900 mg IV, or 1800 mg IV every four weeks (Q4W), wherein the anti-tryptase beta antibody comprises the following six CDRs:
(a) an CDR-H1 comprising the amino acid sequence of DYGMV (SEQ ID NO: 1);
(b) an CDR-H2 comprising the amino acid sequence of FISSGSSTVYYADTMKG (SEQ ID NO: 2);
(c) an CDR-H3 comprising the amino acid sequence of RNYDDWYFDV (SEQ ID NO: 3);
(d) an CDR-L1 comprising the amino acid sequence of SASSSVTYMY (SEQ ID NO: 4);
(e) an CDR-L2 comprising the amino acid sequence of RTSDLAS (SEQ ID NO: 5); and
(f) an CDR-L3 comprising the amino acid sequence of QHYHSYPLT (SEQ ID NO: 6).

19. The method of any one of claims 1-18, wherein the CSU is refractory to antihistamines.

20. The method of claim 19, wherein the CSU is refractory to second-generation H1 antihistamines (sgH1-AHs).

21. The method of claim 20, wherein the patient:
(i) has had a CSU diagnosis for greater than or equal to (≥) 6 months;
(ii) has presence of itch and hives for greater than (>) 6 consecutive weeks at any time prior to treatment despite current use of sgH1-AHs, consistent with standard of care during this time period;
(iii) has received stable doses of sgH1-AHs, consistent with standard of care therapy for CSU, starting at least 14 (−4/+2 days) consecutive days prior to treatment; and/or
(iv) has a Urticaria Activity Score summed over 7 days (UAS7) symptom score of ≥16 during the 7 days prior to the C1D1.

22. The method of any one of claims 1-21, wherein the patient has a UAS7 symptom score of ≥16.

23. The method of any one of claims 1-22, wherein the patient is Chronic Urticaria Index (CU Index®)-positive.

24. The method of any one of claims 1-23, wherein the patient is receiving background sgH1-AH therapy.

25. The method of claim 24, wherein the background sgH1-AH therapy comprises cetirizine 10-40 mg once a day (QD), levocetirizine 5-20 mg QD, fexofenadine 180-720 mg QD, loratadine 10-40 mg QD, desloratadine 5-20 mg QD, rupatadine 10-40 mg QD, or bilastine 20-80 mg QD.

26. The method of any one of claims 1-25, wherein the patient receives a single dose of rescue therapy within a 24-hour period if symptoms worsen.

27. The method of claim 26, wherein the rescue therapy comprises up to 10 mg loratadine or up to 10 mg cetirizine.

28. The method of any one of claims 1-27, wherein the treating results in an improvement from baseline in the patient's UAS7 at Week 12 compared to placebo.

29. The method of any one of claims 1-28, wherein (i) the treatment results in well-controlled urticaria (UAS7 less than or equal to (s) 6 at Week 12); or (ii) the treatment results in a complete response (UAS7=0) at Week 12.

30. A kit comprising an anti-tryptase beta antibody and instructions to administer the anti-tryptase beta antibody to a patient having CSU in accordance with the method of any one of claims 1-29.

31. An anti-tryptase beta antibody for use in treating a patient having CSU, wherein the anti-tryptase beta antibody is for administration to a patient having CSU in a dosing regimen comprising a dosing cycle, wherein the dosing cycle comprises a first dose (C1D1) of the anti-tryptase beta antibody selected from 300 mg SC, 600 mg SC, 900 mg IV, or 1800 mg IV, wherein the anti-tryptase beta antibody comprises the following six CDRs:
(a) an CDR-H1 comprising the amino acid sequence of DYGMV (SEQ ID NO: 1);
(b) an CDR-H2 comprising the amino acid sequence of FISSGSSTVYYADTMKG (SEQ ID NO: 2);
(c) an CDR-H3 comprising the amino acid sequence of RNYDDWYFDV (SEQ ID NO: 3);
(d) an CDR-L1 comprising the amino acid sequence of SASSSVTYMY (SEQ ID NO: 4);
(e) an CDR-L2 comprising the amino acid sequence of RTSDLAS (SEQ ID NO: 5); and
(f) an CDR-L3 comprising the amino acid sequence of QHYHSYPLT (SEQ ID NO: 6).

32. The anti-tryptase beta antibody for use of claim 31, wherein the antibody comprises (a) a heavy chain variable (VH) domain comprising an amino acid sequence having at least 90%, at least 95%, or at least 99% sequence identity to the amino acid sequence of SEQ ID NO: 7; (b) a light chain variable (VL) domain comprising an amino acid sequence having at least 90%, at least 95%, or at least 99% identity to the amino acid sequence of SEQ ID NO: 8; or (c) a VH domain as in (a) and a VL domain as in (b).

33. The anti-tryptase beta antibody for use of claim 32, wherein the VH domain comprises the amino acid sequence of SEQ ID NO: 7.

34. The anti-tryptase beta antibody for use of claim 32, wherein the VL domain comprises the amino acid sequence of SEQ ID NO: 8.

35. The anti-tryptase beta antibody for use of claim 32, wherein the VH domain comprises the amino acid sequence of SEQ ID NO: 7 and the VL domain comprises the amino acid sequence of SEQ ID NO: 8.

36. The anti-tryptase beta antibody for use of any one of claims 31-35, wherein the antibody comprises (a) a heavy chain comprising the amino acid sequence of SEQ ID NO: 9 and (b) a light chain comprising the amino acid sequence of SEQ ID NO: 10.

37. The anti-tryptase beta antibody for use of any one of claims 31-35, wherein the antibody comprises (a) a heavy chain comprising the amino acid sequence of SEQ ID NO: 11 and (b) a light chain comprising the amino acid sequence of SEQ ID NO: 10.

38. The anti-tryptase beta antibody for use of any one of claims 31-37, wherein the C1D1 is 300 mg SC.

39. The anti-tryptase beta antibody for use of any one of claims 31-37, wherein the C1D1 is 600 mg SC.

40. The anti-tryptase beta antibody for use of any one of claims 31-37, wherein the C1D1 is 900 mg IV.

41. The anti-tryptase beta antibody for use of any one of claims 31-37, wherein the C1D1 is 1800 mg IV.

42. The anti-tryptase beta antibody for use of any one of claims 31-41, wherein the dosing cycle further comprises a second dose (C1D2) and a third dose (C1D3) of the anti-tryptase beta antibody, wherein the C1D2 and the C1D3 are each equal to the C1D1.

43. The anti-tryptase beta antibody for use of claim 42, wherein the doses of the dosing cycle are administered to the subject every four weeks (Q4W).

44. The anti-tryptase beta antibody for use of claim 42 or 43, wherein the dosing cycle has a length of about 57 days.

45. The anti-tryptase beta antibody for use of claim 44, wherein the C1D1 is administered on Day 1 of the dosing cycle, the C1D2 is administered on Day 29 (+1 day) of the dosing cycle, and the C1D3 is administered on Day 57 (+1 day) of the dosing cycle.

46. The anti-tryptase beta antibody for use of any one of claims 42-45, wherein the dosing regimen consists of one dosing cycle.

47. An anti-tryptase beta antibody for use in treating a patient having CSU, wherein the anti-tryptase beta antibody is for administration to a patient having CSU in a dosing regimen comprising a dosing cycle, wherein the dosing cycle comprises between two and six doses, wherein a total of about 300 mg to about 21,600 mg of the anti-tryptase antibody is administered SC or IV to the patient in the dosing cycle, and wherein the anti-tryptase beta antibody comprises the following six CDRs:
(a) an CDR-H1 comprising the amino acid sequence of DYGMV (SEQ ID NO: 1);
(b) an CDR-H2 comprising the amino acid sequence of FISSGSSTVYYADTMKG (SEQ ID NO: 2);
(c) an CDR-H3 comprising the amino acid sequence of RNYDDWYFDV (SEQ ID NO: 3);
(d) an CDR-L1 comprising the amino acid sequence of SASSSVTYMY (SEQ ID NO: 4);
(e) an CDR-L2 comprising the amino acid sequence of RTSDLAS (SEQ ID NO: 5); and
(f) an CDR-L3 comprising the amino acid sequence of QHYHSYPLT (SEQ ID NO: 6).

48. An anti-tryptase beta antibody for use in treating a patient having CSU, wherein the anti-tryptase beta antibody is for administration to a patient having CSU in a dosing regimen comprising a dosing cycle, wherein the dosing cycle comprises administering the anti-tryptase beta antibody at a dose selected from 300 mg SC, 600 mg SC, 900 mg IV, or 1800 mg IV every four weeks (Q4W), wherein the anti-tryptase beta antibody comprises the following six CDRs:
(a) an CDR-H1 comprising the amino acid sequence of DYGMV (SEQ ID NO: 1);
(b) an CDR-H2 comprising the amino acid sequence of FISSGSSTVYYADTMKG (SEQ ID NO: 2);
(c) an CDR-H3 comprising the amino acid sequence of RNYDDWYFDV (SEQ ID NO: 3);
(d) an CDR-L1 comprising the amino acid sequence of SASSSVTYMY (SEQ ID NO: 4);
(e) an CDR-L2 comprising the amino acid sequence of RTSDLAS (SEQ ID NO: 5); and
(f) an CDR-L3 comprising the amino acid sequence of QHYHSYPLT (SEQ ID NO: 6).

49. The anti-tryptase beta antibody for use of any one of claims 31-48, wherein the CSU is refractory to antihistamines.

50. The anti-tryptase beta antibody for use of claim 49, wherein the CSU is refractory to sgH1-AHs.

51. The anti-tryptase beta antibody for use of claim 50, wherein the patient:
(i) has had a CSU diagnosis for ≥6 months;
(ii) has presence of itch and hives for >6 consecutive weeks at any time prior to treatment despite current use of sgH1-AHs, consistent with standard of care during this time period;
(iii) has received stable doses of sgH1-AHs, consistent with standard of care therapy for CSU, starting at least 14 (−4/+2 days) consecutive days prior to treatment; and/or
(iv) has a UAS7 symptom score of ≥16 during the 7 days prior to the C1D1.

52. The anti-tryptase beta antibody for use of any one of claims 31-51, wherein the patient has a UAS7 symptom score of ≥16.

53. The anti-tryptase beta antibody for use of any one of claims 31-52, wherein the patient is CU Index®-positive.

54. The anti-tryptase beta antibody for use of any one of claims 31-53, wherein the patient is receiving background sgH1-AH therapy.

55. The anti-tryptase beta antibody for use of claim 54, wherein the background sgH1-AH therapy comprises cetirizine 10-40 mg QD, levocetirizine 5-20 mg QD, fexofenadine 180-720 mg QD, loratadine 10-40 mg QD, desloratadine 5-20 mg QD, rupatadine 10-40 mg QD, or bilastine 20-80 mg QD.

56. The anti-tryptase beta antibody for use of any one of claims 31-55, wherein the patient receives a single dose of rescue therapy within a 24-hour period if symptoms worsen.

57. The anti-tryptase beta antibody for use of claim 56, wherein the rescue therapy comprises up to 10 mg loratadine or up to 10 mg cetirizine.

58. The anti-tryptase beta antibody for use of any one of claims 31-57, wherein the treating results in an improvement from baseline in the patient's UAS7 at Week 12 compared to placebo.

59. The anti-tryptase beta antibody for use of any one of claims 31-58, wherein (i) the treatment results in well-controlled urticaria (UAS7≤6 at Week 12); or (ii) the treatment results in a complete response (UAS7=0) at Week 12.

60. Use of an anti-tryptase beta antibody in the manufacture of a medicament for treating a patient having CSU, wherein the medicament is for administration to a patient having CSU in a dosing regimen comprising a dosing cycle, wherein the dosing cycle comprises a first dose (C1D1) of the anti-tryptase beta antibody selected from 300 mg SC, 600 mg SC, 900 mg IV, or 1800 mg IV, wherein the anti-tryptase beta antibody comprises the following six CDRs:
(a) an CDR-H1 comprising the amino acid sequence of DYGMV (SEQ ID NO: 1);
(b) an CDR-H2 comprising the amino acid sequence of FISSGSSTVYYADTMKG (SEQ ID NO: 2);
(c) an CDR-H3 comprising the amino acid sequence of RNYDDWYFDV (SEQ ID NO: 3);
(d) an CDR-L1 comprising the amino acid sequence of SASSSVTYMY (SEQ ID NO: 4);
(e) an CDR-L2 comprising the amino acid sequence of RTSDLAS (SEQ ID NO: 5); and
(f) an CDR-L3 comprising the amino acid sequence of QHYHSYPLT (SEQ ID NO: 6).

61. Use of an anti-tryptase beta antibody in the manufacture of a medicament for treating a patient having CSU, wherein the medicament is for administration to a patient having CSU in a dosing regimen comprising a dosing cycle, wherein the dosing cycle comprises between two and six doses, wherein a total of about 300 mg to about 21,600 mg of the anti-tryptase antibody is administered SC or IV to the patient in the dosing cycle, wherein the anti-tryptase beta antibody comprises the following six CDRs:
(a) an CDR-H1 comprising the amino acid sequence of DYGMV (SEQ ID NO: 1);
(b) an CDR-H2 comprising the amino acid sequence of FISSGSSTVYYADTMKG (SEQ ID NO: 2);
(c) an CDR-H3 comprising the amino acid sequence of RNYDDWYFDV (SEQ ID NO: 3);
(d) an CDR-L1 comprising the amino acid sequence of SASSSVTYMY (SEQ ID NO: 4);
(e) an CDR-L2 comprising the amino acid sequence of RTSDLAS (SEQ ID NO: 5); and
(f) an CDR-L3 comprising the amino acid sequence of QHYHSYPLT (SEQ ID NO: 6).

62. Use of an anti-tryptase beta antibody in the manufacture of a medicament for treating a patient having CSU, wherein the medicament is for administration to a patient having CSU in a dosing regimen comprising a dosing cycle, wherein the dosing cycle comprises administering the anti-tryptase beta antibody at a dose selected from 300 mg SC, 600 mg SC, 900 mg IV, or 1800 mg IV every four weeks (Q4W), wherein the anti-tryptase beta antibody comprises the following six CDRs:
(a) an CDR-H1 comprising the amino acid sequence of DYGMV (SEQ ID NO: 1);
(b) an CDR-H2 comprising the amino acid sequence of FISSGSSTVYYADTMKG (SEQ ID NO: 2);
(c) an CDR-H3 comprising the amino acid sequence of RNYDDWYFDV (SEQ ID NO: 3);
(d) an CDR-L1 comprising the amino acid sequence of SASSSVTYMY (SEQ ID NO: 4);
(e) an CDR-L2 comprising the amino acid sequence of RTSDLAS (SEQ ID NO: 5); and
(f) an CDR-L3 comprising the amino acid sequence of QHYHSYPLT (SEQ ID NO: 6).
